(19) United States
(12) Patent Application Publication
(10) Pub. No.: US 2026/0217983 A1
(43) Pub. Date: Jul. 30, 2026

(54) SULFONIUM SALT PHOTOINITIATORS AND METHODS OF LED CURING A COMPOSITION WITH SAID SULFONIUM SALT PHOTOINITIATORS

(71) Applicants: ARKEMA FRANCE, Puteaux (FR); CENTRE NATIONAL DE LA RECHERCHE SCIENTIFIQUE, Paris (FR); UNIVERSITE DE HAUTE - ALSACE, Mulhouse Cedex (FR)

(72) Inventors: Petr SEHNAL, Wetherby Yorkshire (GB); Richard PLENDERLEITH, Wetherby Yorkshire (GB); Kelly SQUIRES, Wetherby Yorkshire (GB); Elodie SPRICK, Rixheim (FR); Jacques LALEVEE, Mulhouse (FR); Jean-Michel BECHT, Tagolsheim (FR); Kangtai REN, Exton, PA (US); Tejaswi NORI, Exton, PA (US); Jeffrey KLANG, Exton, PA (US)

(73) Assignees: ARKEMA FRANCE, Puteaux (FR); CENTRE NATIONAL DE LA RECHERCHE SCIENTIFIQUE, Paris (FR); UNIVERSITE DE HAUTE - ALSACE, Mulhouse Cedex (FR)

(21) Appl. No.: 19/141,746

(22) PCT Filed: Dec. 27, 2023
(86) PCT No.: PCT/EP2023/087817

§ 371 Date: Jun. 20, 2025

(30) Foreign Application Priority Data
Dec. 28, 2022 (FR) FR2214580
Dec. 28, 2022 (FR) FR2214581

(51) Int. Cl. C09D 4/00; B29C 64/129; B29C 64/30; B29C 71/02; B29K 33/00; B29K 63/00; B29K 105/00; B33Y 10/00; B33Y 40/20; B33Y 70/10; C07D 335/16; C08F 22/10; C08F 22/20; C08G 59/68; C08K 5/17; C09D 7/48; C09D 7/63; C09D 135/02; C09D 163/00

(52) U.S. Cl. CPC C09D 4/00; B29C 64/129; B29C 64/30; B29C 71/02; B33Y 10/00; B33Y 40/20; B33Y 70/10; C07D 335/16; C08F 22/1006; C08F 22/20; C08G 59/687; C09D 7/48; C09D 7/63; C09D 135/02; C09D 163/00; B29K 2033/08; B29K 2063/00; B29K 2105/0005; C08K 5/17

(57) ABSTRACT

The present invention concerns a method of curing a composition comprising a cationically-polymerizable compound and a photoinitiator of formula (I):

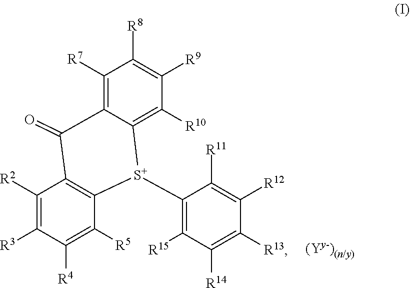

the method comprising irradiating the composition with at least one light source having a maximum output wavelength in the range of 350 to 460 nm, the obtained cured product, a method for the preparation of a 3D-printed article comprising said method and the obtained 3D printed article.

The present invention relates to a method of curing a composition comprising a cationically-polymerizable compound and a photoinitiator, the method comprising irradiating the composition with at least one light source having a maximum output wavelength in the range of 350 to 460 nm, the obtained cured product, a method for the preparation of a 3D-printed article comprising said method and the obtained 3D printed article.

Stereolithography (SLA or SL) is a form of 3D printing technology used for creating 3D articles or parts thereof in a layer by layer fashion using photochemical processes by which light causes polymerizable compound to cross-link together to form polymers.

(Meth)acrylate stereolithographic resins have the disadvantages of high volume shrinkage and high shrinkage stress.

Hybrid systems, containing both radically-curable (meth)acrylates and cationically-curable epoxides, overcame the limitations of (meth)acrylate-based resins, and thus became one of most successful innovations in SL resins history. The hybrid system builds interpenetration polymer network (IPN) simultaneously, eliminates the oxygen inhibition of (meth)acrylates and reduces the viscosity, thus resulting in an enhanced process speed, an easier cleaning of parts, an improved green strength that makes the part support much stronger and eliminating the curl of distortion during building.

Cationic cure in hybrid systems involves ring opening polymerization, epoxides being mostly used. This reduces the volume shrinkage, but also decreases shrinkage stress due to slower cure speed than (meth)acrylate cure during green part building. One of the important benefits of cationic cure is that the green parts can be post cured, the so-called dark cure of cationic polymerization continues during aging, and the mechanical properties are fully developed after days.

3D printers equipped with longer wavelength LED lamps are tremendously growing due to high penetration depth of LED light, low health hazard, and cost efficiency.

For a typical hybrid photocurable 3D resin (requiring both radical cure and cationic cure), both radical photoinitiator and cationic photoinitiator are needed. These photoinitiators absorb photons upon irradiation with light and form either radical species or cationic species from their excited state, which initiates consecutive reactions for photopolymerization of UV curable 3D resins.

Among photoinitiators for LED curable 3D resin, many commercial type I and type II radical initiators exhibit a good match between their absorption lines and the LED emission spectrum of 3D printers, and provide efficient photoreactivity and high cure speed to radically curable monomers and/or oligomers. For example, acylphosphine oxides have a red shifted absorption band in the range of 350 to 420 nm which is compatible with many LED light emission ranges, and are thus considered as efficient radical photoinitiators.

However, long wavelength photoinitiating systems for cationic photopolymerization have become a bottleneck to develop LED curable cationic systems and hybrid systems for 3D LED printers. Current commercial iodonium and sulfonium salts exhibit short wavelength light absorption around 220 to 320 nm, and have a very limited overlap with LED light emission spectra of 3D printer.

Extending the wavelength range of cationic photoinitiating systems has attracted growing interest in recent years. Three typical approaches have been developed:
1) Designing new onium salts with long wavelength absorbing chromophores, such as sulfonium salt Omnicat 550;
2) Photosensitizing conventional onium salts with various visible light photosensitizers such as SpeedCure® CPTX;
3) Promoting cationic cure with radical initiators, such as SpeedCure® BKL.

To date, all three approaches have been successfully applied to extend the light absorption of iodonium salts to longer wavelengths and have become very attractive systems for academic fields. Unfortunately, iodonium salts have quite poor thermal stability. The short shelf life of LED curable products, developed from them, has limited their industrial applications including 3D printing.

Sulfonium salts usually have better thermal stability and longer wavelength absorption, match LED emission spectrum better than iodonium salts, hence becoming a favorite cationic photoinitiator for many industrial applications. However, commercial sulfonium salts still have a big gap to match the wavelength of LED lamps over 350 nm. First of all, it is well reported that sulfonium salts are difficult or unfavorable to sensitize by photosensitizers, since the free energy barrier of electron transfer between them and most photosensitizers is much higher than the one between iodonium salts and photosensitizers. Very few photosensitizers work well, such as 9,10-dibutoxyanthracene (UVS-1101 from Nagase) with a particularly effective dose range of 0.4-0.6%. Secondly, it is also well known that commercial sulfonium salts are difficult to promote by radical photoinitiators as well. Since their half-wave reduction potential E_{1/2}^{red}(On+) (−1.2 V vs. SCE) is much lower than that of iodonium salts (−0.2 V vs SCE), sulfonium salts can't oxidize free radicals generated from radical photoinitiators, and are not able to facilitate the electron transfer process to produce cation initiating species. Among the three strategies to extend sulfonium salts to longer wavelengths, two of them have had quite limited success so far. The most successful choice to date is modifying the structure of the sulfonium salt by introducing chromophore groups. Irgacure PAG 290 [⁻B(C₆F₅)₄ salt]or PAG 270 (⁻PF₆ salt) from BASF, Omnicat 550 or Omnicat 650 from IGM are sulfonium salts of this kind that have enjoyed commercial success over the past years, but these sulfonium salts are not red-shifted enough to successfully cure cationic or hybrid resins with LED lamps over 350 nm.

There is a thus need for photoinitiating systems for cationic photopolymerization, which are usable at long wavelengths and in particular within the LED emission spectrum.

According to a first object, the invention relates to a method of curing a composition comprising a cationically-polymerizable compound and a photoinitiator of formula (I):

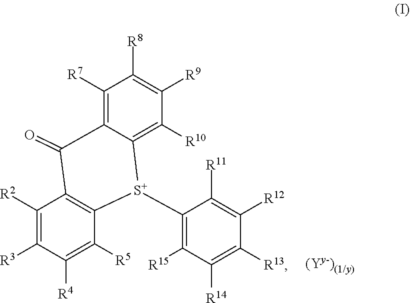

wherein:
Y is an anion, the valency of which is y,
either R¹² and R¹³ are linked with each other so that the

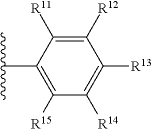

group represents

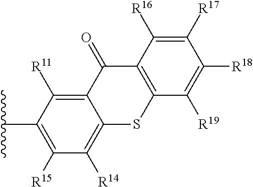

wherein:
   R¹⁶, R¹⁷, R¹⁸ and R¹⁹ are independently chosen among H, a halogen, a (C₁-C₆) linear or branched alkyl group, a (C₁-C₆) linear or branched alkoxy group, a —O—(CH₂)ᵢ—COOR²⁸ or —(CH₂)ᵢ—CH—(COOR²⁸)₂ group wherein i is 1 or 2 and R²⁸ is H or a (C₁-C₄) linear or branched alkyl group,
   R¹¹, R¹⁴ and R¹⁵ are independently H, a halogen, a (C₁-C₆) linear or branched alkyl group, a (C₁-C₆) linear or branched alkoxy group and —S-Ph-C(═O)-Ph,
or R¹² and R¹³ are not linked with each other, and
R¹¹, R¹² and R¹³ are independently chosen from H, a halogen, a (C₁-C₆) linear or branched alkyl group, a (C₁-C₆) linear or branched alkoxy group, a pyrrolidin-1-yl, a -L-Ph¹ group wherein L is a single bond, CH₂ or O and Ph¹ is a phenyl group optionally substituted by one or several substituents chosen from a halogen, a (C₁-C₆) linear or branched alkyl group and a (C₁-C₆) linear or branched alkoxy group, provided that at least one R¹¹, R¹² and R¹³ is a -L-Ph¹ group, and
R¹⁴ and R¹⁵ are independently H, a halogen, a (C₁-C₆) linear or branched alkyl group, a (C₁-C₆) linear or branched alkoxy group and —S-Ph-C(═O)-Ph,
R³ is chosen from H, a halogen, a (C₁-C₆) linear or branched alkyl group, a (C₁-C₆) linear or branched alkoxy group, a —O—(CH₂)ₗ—COOR³¹ group and a —(CH₂)ₗ—CH—(COOR³¹)₂ group, wherein l is 1 or 2 and R³¹ is H or a (C₁-C₄) linear or branched alkyl group,
R², R⁴, R⁵, R⁷, R⁸, R⁹ and R¹⁰ are independently chosen from H, a halogen, a (C₁-C₆) linear or branched alkyl group, a (C₁-C₆) linear or branched alkoxy group and a —O—(CH₂)ₘ—COOR³² or —(CH₂)ₘ—CH—(COOR³²)₂ group wherein m is 1 or 2 and R³² is H or a (C₁-C₄) linear or branched alkyl group,
   the method comprising irradiating the composition with at least one light source having a maximum output wavelength in the range of 350 to 460 nm.

Photoinitiators are generally divided into two classes, depending on their mode of action: radical photoinitiators and cationic photoinitiators. When cationic photoinitiators are irradiated, they undergo homolytic or heterolytic bond cleavage forming fragments that decompose or react further to give a Brønsted or Lewis acid. The generated acid then initiates the polymerization. Radical photoinitiators can adopt two different modes of action, and are classified by mode of action as Norrish Type I and Norrish Type II photoinitiators. As used herein, the term “activity” with reference to Norrish Type I and Norrish Type II activity is intended to relate to Norrish photoinitiation and analogous reactions. For instance, a photoinitiator having Norrish Type I activity would be a photoinitiator characterized by a cleavage reaction into two radical fragments of the original photoinitiator on exposure to light. For an initiator having Norrish Type II activity, exposure to light causes the abstraction of an atom, such as hydrogen, to generate the radical.

The sulfonium salts of formula (I) are Type I radical photoinitiators, advantageously red-shifted up to 420 nm wavelength thus matching the majority of commercial LED lamps, and rendering possible the cure of cationic systems or hybrid systems on 3D printer equipped LED lamps.

Compared to conventional photoinitiators, the sulfonium salts of formula (I) allow obtaining identical or faster cure speed than that obtained with iodonium salts in combination with a thioxanthone sensitizer, faster cure speed than that obtained with commercial sulfonium salts on the market and provide superior green strength of printed parts. The photoinitiators of formula (I) advantageously show high cure speeds, notably in purely cationic (for example epoxy and/or oxetane-based) and hybrid (for example epoxy/(meth)acrylate based) formulations when cured with light sources from 350 to 460 nm. In hybrid formulations, high conversion of both types of monomers without phase separation is achieved when sulfonium salts of formula (I) are used. This results in high strength and reduced shrinkage.

The photoinitiators of formula (I) exhibit acceptable yellowing and/or photobleaching characteristics which are important for applications in printing inks and additive manufacturing. The low yellowing characteristics can be measured by the Colour index ‘b’ value on cured films.

Formulations comprising monomers and the photoinitiators of formula (I) advantageously provide superior thermal stability for LED curable cationic or hybrid products for 3D printing.

The curable composition may comprise 0.05% to 10%, in particular 0.1% to 5%, more particularly 0.5 to 2%, by weight of photoinitiator of formula (I) based on the total weight of the curable composition.

The method according to the invention comprises irradiating the composition with at least one light source having a maximum output wavelength in the range of 350 to 460 nm, preferably from 365 to 450 nm, notably from 380 to 430 nm, even more preferably of 385 nm or 395 nm or 405 nm or 420 nm.

The light source is generally a light-emitting diode (LED), or a broadband lamp with an optical filter that limits emission to wavelengths in the range of 350 to 460 nm.

The preferred embodiments hereafter can be considered singly of combined with each other when applicable, and can be applied to formula (I) and any one of the formulae described hereafter, in particular any one of formulae (II) to (XV), when applicable:
the (C₁-C₆) linear or branched alkyl group is a (C₁-C₃) linear or branched alkyl group, preferably methyl (Me), ethyl (Et), isopropyl (iPr) or n-propyl (nPr),
the (C₁-C₆) linear or branched alkoxy group is a (C₁-C₃) linear or branched alkoxy group, preferably —OMe, OEt, OiPr, -OnPr,
the halogen is Cl or F,
R¹² and R¹³ are linked with each other so that the group

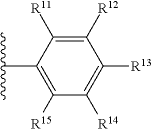

represents

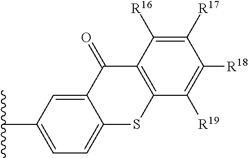

R⁷, R⁸, R⁹ and R¹⁰ represent H,
R⁸ and/or R¹³ are not methyl, and/or
R³ and R⁸ are not Me, and preferably none of R², R³, R⁴, R⁵, R⁷, R⁸, R⁹ and R¹⁰ is iPr,
R² and R⁷ are not a halogen,
none of R³, R⁵, R⁸ and R¹⁰ is Et,
R¹¹, R¹⁴ and R¹⁵ are independently H, a halogen, a (C₁-C₆) linear or branched alkyl group or a (C₁-C₆) linear or branched alkoxy group,
at least one group among R¹² and R¹³ is a -L-Ph¹ group, preferably one group among R¹² and R¹³ is a -L-Ph¹ group,
L is a single bond,
Ph¹ is a phenyl group optionally substituted by one substituent chosen from a halogen, a (C₁-C₆) linear or branched alkyl group and a (C₁-C₆) linear or branched alkoxy group, preferably by a (C₁-C₆) linear or branched alkoxy group, most preferably by a methoxy group, and/or
when L is a single bond, then Ph¹ is a phenyl group substituted by at least one (C₁-C₆) linear or branched alkoxy group.

In any formula described in the present application, the anion Y^{y-} is preferably chosen from halogenide (F⁻, Cl⁻, Br⁻, I⁻), HSO₄⁻, SO₄²⁻, ClO₄⁻, BF₄⁻, PF₆⁻, AsF₆⁻, SbF₆⁻, SbF₅(OH)⁻, SbF₄(OH)₂⁻, BPh₄⁻, B(C₆F₅)₄⁻, Al[OC(CF₃)₃]₄⁻, CH₃COO⁻, CH₃SO₃⁻, CH₃C₆H₄SO₃⁻, CF₃COO⁻, CF₃SO₃⁻, N(CF₃SO₃)₂⁻, or B[C₆H₃(CF₃)₂]₄⁻, and is most preferably chosen from PF₆⁻, SbF₆⁻ and B(C₆F₅)₄⁻.

In a first alternative, in formula (I), R¹² and R¹³ are linked with each other so that the group

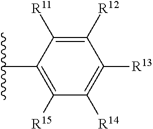

represents

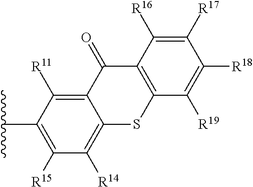

and the photoinitiators match formula (IV):

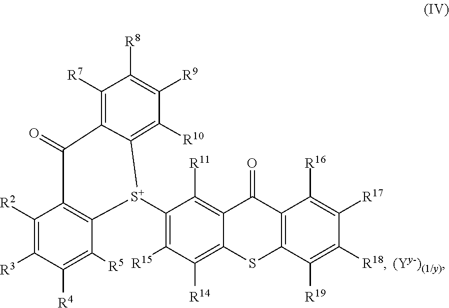

wherein R², R³, R⁴, R⁵, R⁷, R⁸, R⁹, R¹⁰, R¹¹, R¹⁴, R¹⁵, R¹⁶, R¹⁷, R¹⁸, R¹⁹, Y and y are as defined above.

Hence, in formula (IV):
R¹⁶, R¹⁷, R¹⁸ and R¹⁹ are independently chosen among H, a halogen, a (C₁-C₆) linear or branched alkyl group, a (C₁-C₆) linear or branched alkoxy group, a —O—(CH₂)ᵢ—COOR²⁸ or —(CH₂)ᵢ—CH—(COOR²⁸)₂ group wherein i is 1 or 2 and R²⁸ is H or a (C₁-C₄) linear or branched alkyl group,
R¹¹, R¹⁴, R¹⁵ are independently H, a halogen, a (C₁-C₆) linear or branched alkyl group, a (C₁-C₆) linear or branched alkoxy group and —S-Ph-C(═O)-Ph.

The preferred embodiments hereafter can be considered singly of combined with each other when applicable, and can be applied to formula (IV), and, when applicable, also to formula (V) or (V′) described hereafter:
R⁸ represents H,
R⁷, R⁸, R⁹ and R¹⁰ represent H,
R¹¹, R¹⁴ and R¹⁵ represent H,
R⁴ is H,
R², R⁴, R⁷, R⁸, R⁹ and R¹⁰ represent H, R⁵ is chosen from H, a halogen, a (C₁-C₆) linear or branched alkyl group, and R³ is chosen from a halogen, a (C₁-C₆) linear or branched alkyl group, a (C₁-C₆) linear or branched alkoxy group, a —O—(CH₂), —COOR³¹ group and a —(CH₂)ₗ—CH—(COOR³¹)₂ group, wherein l is 1 or 2 and R³¹ is H or a (C₁-C₄) linear or branched alkyl group, R³ being preferably chosen from a halogen, a (C₁-C₆) linear or branched alkyl group and a (C₁-C₆) linear or branched alkoxy group, R³ being most preferably a (C₁-C₆) linear or branched alkyl group,
or
R³, R⁴, R⁷, R⁸, R⁹ and R¹⁰ are H and R² and R⁵ are independently chosen from a halogen, a (C₁-C₆) linear or branched alkyl group, a (C₁-C₆) linear or branched alkoxy group and a —O—(CH₂)ₘ-COOR³² or —(CH₂)ₘ-CH—(COOR³²)₂ group wherein m is 1 or 2 and R³² is H or a (C₁-C₄) linear or branched alkyl group, R² and R⁵ being preferably independently chosen from a halogen, a (C₁-C₆) linear or branched alkyl group and a (C₁-C₆) linear or branched alkoxy group, R² and R⁵ being most preferably independently chosen from a halogen and a (C₁-C₆) linear or branched alkoxy group, and/or
R¹⁶, R¹⁸, R¹¹, R¹⁴ and R¹⁵ represent H, R¹⁹ is chosen from H, a halogen, a (C₁-C₆) linear or branched alkyl group and R¹⁷ is chosen from a halogen, a (C₁-C₆) linear or branched alkyl group, a (C₁-C₆) linear or branched alkoxy group, a —O—(CH₂)ᵢ—COOR²⁸ or —(CH₂)ᵢ—CH—(COOR²⁸)₂ group wherein i is 1 or 2 and R²⁸ is H or a (C₁-C₄) linear or branched alkyl group, R¹⁷ being preferably chosen from a halogen, a (C₁-C₆) linear or branched alkyl group and a (C₁-C₆) linear or branched alkoxy group, R¹⁷ being most preferably a (C₁-C₆) linear or branched alkyl group,
or
R¹⁷, R¹⁸, R¹¹, R¹⁴ and R¹⁵ are H and R¹⁶ and R¹⁹ are independently chosen from a halogen, a (C₁-C₆) linear or branched alkyl group, a (C₁-C₆) linear or branched alkoxy group and a —O—(CH₂)ᵢ—COOR²⁸ or —(CH₂)ᵢ—CH—(COOR²⁸)₂ group wherein i is 1 or 2 and R²⁸ is H or a (C₁-C₄) linear or branched alkyl group, R¹⁶ and R¹⁹ being preferably independently chosen from a halogen, a (C₁-C₆) linear or branched alkyl group and a (C₁-C₆) linear or branched alkoxy group, R¹⁶ and R¹⁹ being most preferably independently chosen from a halogen and a (C₁-C₆) linear or branched alkoxy group.

Preferred photoinitiators of formula (IV) are those having formula (1), (5), (6), (40), (44), (45), (46), (47), (48) or (49), most preferably formula (1) or (5):

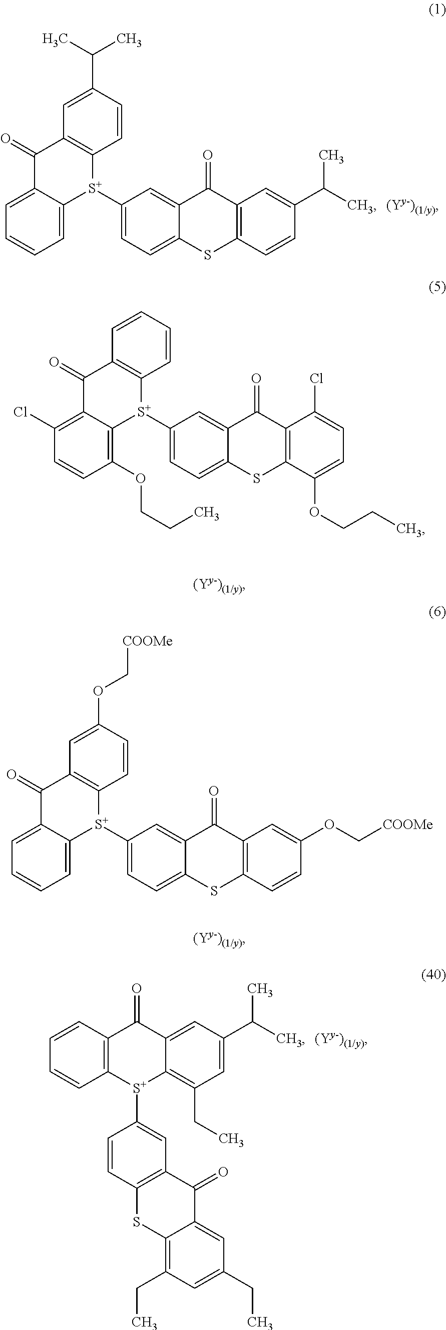

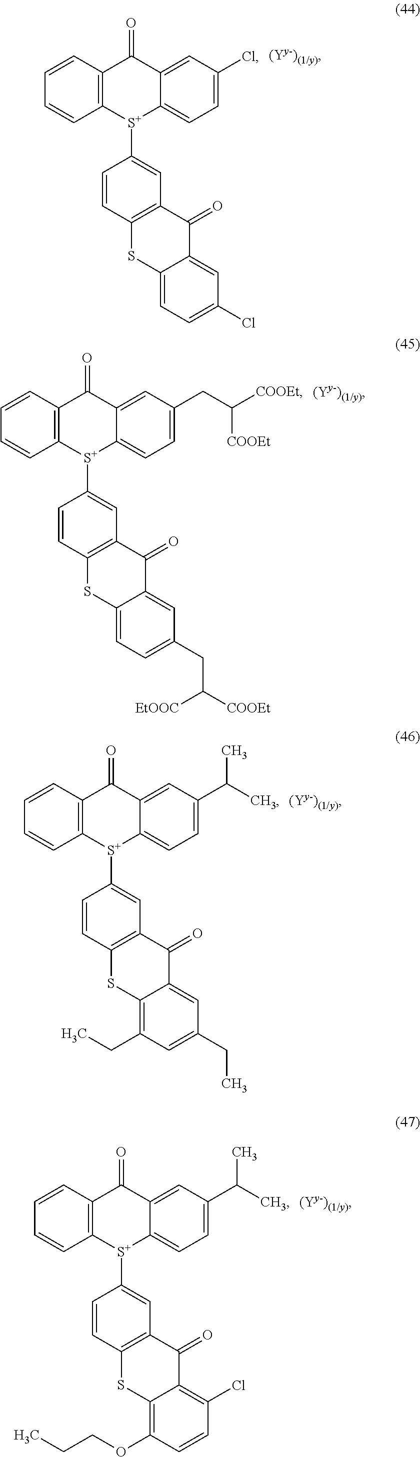

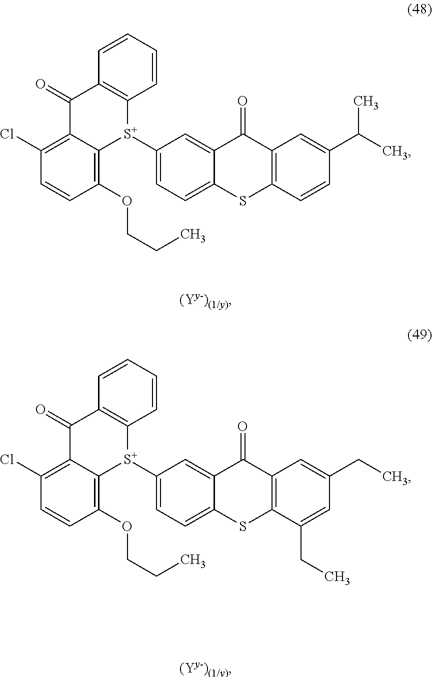

wherein Y and y are as defined above.

Preferably, in formula (IV):

R⁷ and R¹¹ are identical,
R⁹ and R¹⁵ are identical,
R¹⁰ and R¹⁴ are identical,
R² and R¹⁶ are identical,
R³ and R¹⁷ are identical,
R⁴ and R¹⁸ are identical, and
R⁵ and R¹⁹ are identical, and the photoinitiator has formula (V):

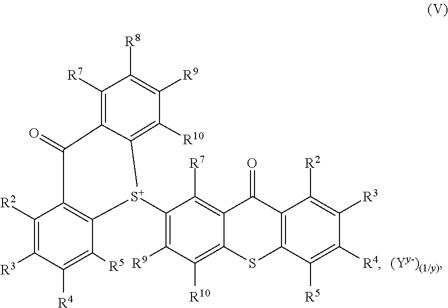

wherein
R⁷, R⁹ and R¹⁰ are independently H, a halogen, a (C₁-C₆) linear or branched alkyl group or a (C₁-C₆) linear or branched alkoxy group,
R², R³, R⁴ and R⁵ are independently chosen among H, a halogen, a (C₁-C₆) linear or branched alkyl group, a (C₁-C₆) linear or branched alkoxy group, a —O—(CH₂)ₘ-COOR³² or —(CH₂)ₘ-CH—(COOR³²)₂ group wherein m is 1 or 2 and R³² is H or a (C₁-C₄) linear or branched alkyl group, and
R⁸, Y and y are as defined above.

Preferred photoinitiators of formula (V) are those having formula (1), (5), (6), (40), (44) or (45) as defined above, most preferably formula (1) or (5).

Preferably, R⁸ is H and the photoinitiator has formula (V′):

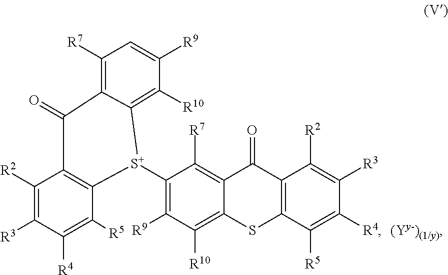

wherein R², R³, R⁴, R⁵, R¹, R⁹, R¹⁰, Y and y are as defined above. Advantageously, and as detailed hereafter, these compounds may be prepared under mild conditions. A compound of formula (V′) wherein
Y is an anion, the valency of which is y,
R³, R⁴, R⁷, R⁹ and R¹⁰ are H, and
R² and R⁵ are independently chosen from a halogen, a (C₁-C₆) linear or branched alkyl group, a (C₁-C₆) linear or branched alkoxy group and a —O—(CH₂)ₘ-COOR³² or —(CH₂)ₘ—CH—(COOR³²)₂ group wherein m is 1 or 2 and R³² is H or a (C₁-C₄) linear or branched alkyl group, R² and R⁵ being preferably independently chosen from a halogen, a (C₁-C₆) linear or branched alkyl group and a (C₁-C₆) linear or branched alkoxy group, R² and R⁵ being most preferably independently chosen from a halogen and a (C₁-C₆) linear or branched alkoxy group
   is an object of the invention.

Preferred photoinitiators of formula (V′) are those having formula (1), (5), (6), (40), (44) or (45) as defined above, most preferably formula (1) or (5).

In a second alternative of formula (1), R¹² and R¹³ are not linked with each other, so that the photoinitiators have formula (VII):

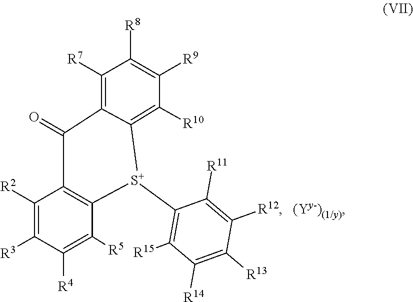

wherein R², R³, R⁴, R⁵, R⁷, R⁸, R⁹, R¹⁰, R¹¹, R¹², R¹³, R¹⁴, R¹⁵, Y and y are as defined above.

Hence, in formula (VII):
R¹¹, R¹² and R¹³ are independently chosen from H, a halogen, a (C₁-C₆) linear or branched alkyl group, a (C₁-C₆) linear or branched alkoxy group, a pyrrolidin-1-yl, a -L-Ph¹ group wherein L is a single bond, CH₂ or O and Ph¹ is a phenyl group optionally substituted by one or several substituents chosen from a halogen, a (C₁-C₆) linear or branched alkyl group and a (C₁-C₆) linear or branched alkoxy group, provided that at least one R¹¹, R¹² and R¹³ is a -L-Ph¹ group, and
R¹⁴ and R¹⁵ are independently H, a halogen, a (C₁-C₆) linear or branched alkyl group, a (C₁-C₆) linear or branched alkoxy group and —S-Ph-C(═O)-Ph.

The preferred embodiments hereafter can be considered singly of combined with each other when applicable, and can be applied to formula (VII), and, when applicable, also to formula (VIII) described hereafter:
R⁷, R⁸, R⁹ and R¹⁰ represent H,
R⁴ is H,
R³ is chosen from a halogen, a (C₁—C) linear or branched alkyl group, a (C₁—C) linear or branched alkoxy group, a —O—(CH₂)ᵣ-COOR³¹ group and a —(CH₂)ᵣ-CH—(COOR³¹)₂ group, wherein l is 1 or 2 and R³¹ is H or a (C₁-C₄) linear or branched alkyl group, R³ being preferably chosen from a halogen, a (C₁—C) linear or branched alkyl group and a (C₁—C) linear or branched alkoxy group, R³ being most preferably a (C₁-C₆) linear or branched alkyl group, R², R⁴, R⁷, R⁸, R⁹ and R¹⁰ represent H and R⁵ is chosen from H, a halogen, a (C₁-C₆) linear or branched alkyl group,
or
R² and R⁵ are independently chosen from a halogen, a (C₁-C₆) linear or branched alkyl group, a (C₁-C₆) linear or branched alkoxy group and a —O—(CH₂)ₘ-COOR³² or —(CH₂)ₘ—CH—(COOR³²)₂ group wherein m is 1 or 2 and R³² is H or a (C₁-C₄) linear or branched alkyl group, R² and R⁵ being preferably independently chosen from a halogen, a (C₁-C₆) linear or branched alkyl group and a (C₁-C₆) linear or branched alkoxy group, R² and R⁵ being most preferably independently chosen from a halogen and a (C₁-C₆) linear or branched alkoxy group, and R³, R⁴, R⁷, R⁸, R⁹ and R¹⁰ are H,
one group among R¹¹, R¹² and R¹³ is a -L-Ph¹ group wherein L is a single bond, CH₂ or O and Ph¹ is a phenyl group optionally substituted by one or several substituents chosen from a halogen, a (C₁-C₆) linear or branched alkyl group and a (C₁-C₆) linear or branched alkoxy group, and the remaining two other groups among R¹¹, R¹² and R¹³ are independently chosen from H, a halogen, a (C₁-C₆) linear or branched alkyl group, a (C₁-C₆) linear or branched alkoxy group, a pyrrolidin-1-yl, the remaining two other groups among R¹¹, R¹² and R¹³ being preferably chosen from H, a halogen, a (C₁-C₆) linear or branched alkyl group, a (C₁-C₆) linear or branched alkoxy group, the remaining two other groups among R¹¹, R¹² and R¹³ being most preferably chosen from H and a (C₁-C₆) linear or branched alkoxy group,
L is a single bond, and/or
Ph¹ is a phenyl group optionally substituted by one substituent chosen from a halogen, a (C₁-C₆) linear or branched alkyl group and a (C₁-C₆) linear or branched alkoxy group, preferably by a (C₁-C₆) linear or branched alkoxy group, most preferably by a methoxy group.

Preferred photoinitiators are those of formula (12), (16), (26), (28), (32) and (33), the most preferred being formula (26), (28), (32) and (33):

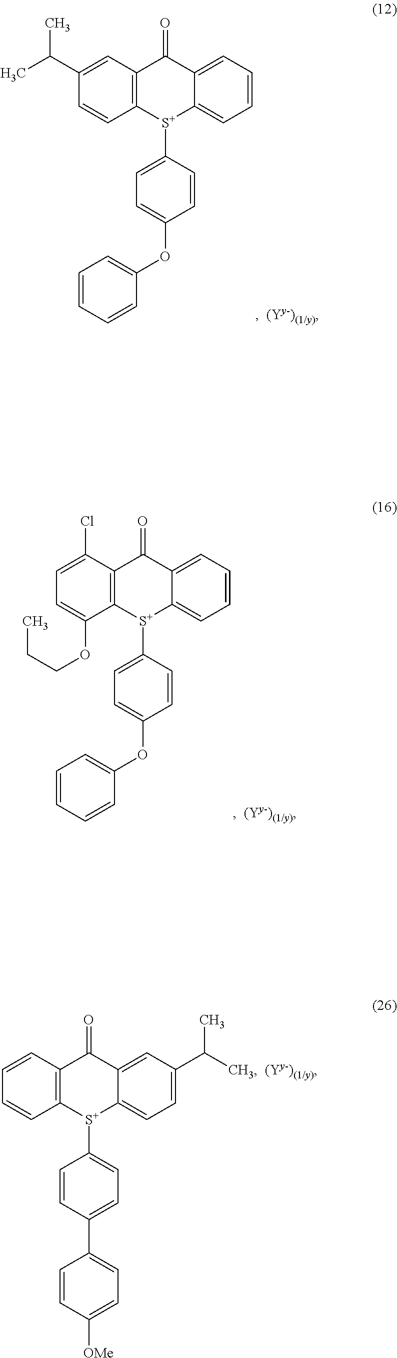

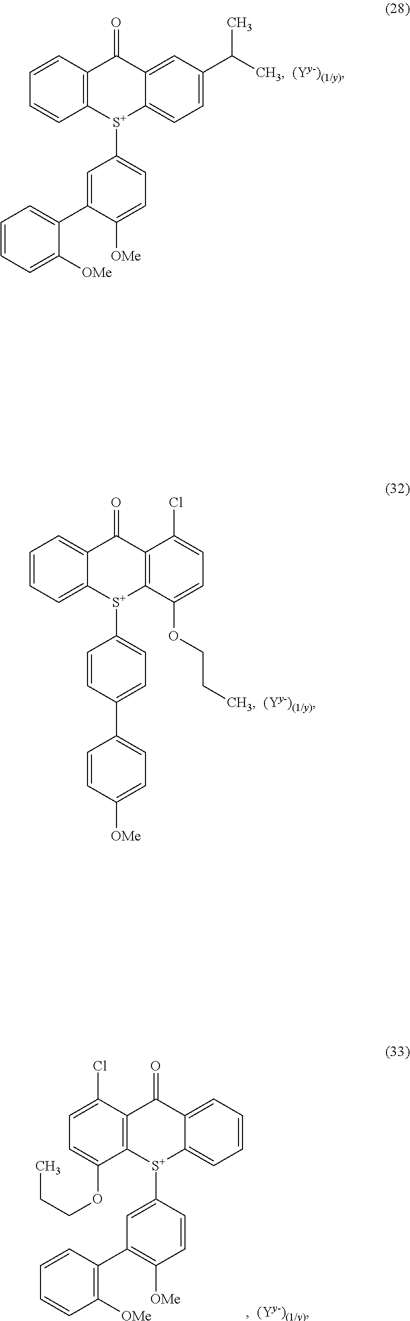

wherein Y and y are as defined above.

In an embodiment, in formula (VII), L is a single bond, and the photoinitiators have formula (VIII):

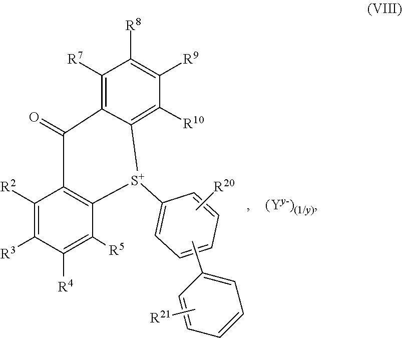

wherein:
R², R³, R⁴, R⁵, R⁷, R⁸, R⁹, R¹⁰, Y and y are as defined above, and
R²⁰ and R²¹ are independently chosen from H, a halogen, a (C₁-C₆) linear or branched alkyl group, a (C₁-C₆) linear or branched alkoxy group, preferably chosen from H and a (C₁-C₆) linear or branched alkoxy group, most preferably chosen from H and OMe.

A compound of formula (VIII) is an object of the invention.

Preferably, in formula (VIII), R²⁰ is independently chosen from H, a halogen, a (C₁-C₆) linear or branched alkyl group, a (C₁-C₆) linear or branched alkoxy group, preferably chosen from H and a (C₁-C₆) linear or branched alkoxy group, most preferably chosen from H and OMe, and R²¹ is a (C₁-C₆) linear or branched alkoxy group, preferably OMe.

Preferred photoinitiators of formula (VIII) are those of formula (26), (28), (32) and (33).

The photoinitiators of formula (I) may be prepared by a method comprising the steps of:

b) reacting a compound of formula (XXI):

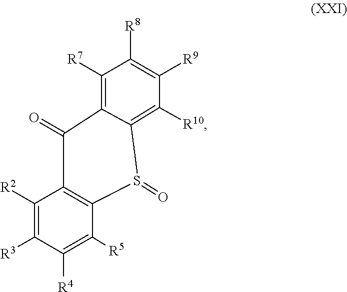

wherein R², R³, R⁴, R⁵, R⁷, R⁸, R⁹, R¹⁰ are as defined above,
with a compound of formula (XXII):

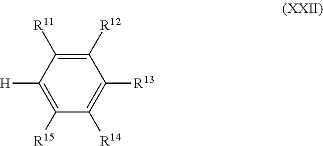

wherein R¹¹, R¹², R¹³, R¹⁴ and R¹⁵ are as defined above,

to form a photoinitiator of formula (I),

in the presence of an activating agent,
whereby a photoinitiator of formula (I) is obtained,
c) when a photoinitiator of formula (I) is desired, wherein Y^{y-} differs from the one obtained at step b), carrying out an ion exchange reaction with a salt comprising Y′^{y-} as anion, or an acid, the base of which is Y′^{y-}, to obtain a photoinitiator of formula (I) wherein Y′^{y-} has the same definition than Y^{y-} as defined above but differs from Y^{y-}obtained at step b).

At step b), the activating agent is typically chosen from trifluoromethanesulfonic anhydride ((CF₃SO₂)₂O, Tf₂O), methanesulfonic anhydride ((CH₃SO₂)₂O), trifluoroacetic anhydride ((CF₃CO)₂O), acetic anhydride ((CH₃CO)₂O), aluminium chloride (AlCl₃) and phosphorus pentoxide (P₂O₅), the activating agent being optionally used in combination with a strong Brønsted acid, such as trifluoromethanesulfonic acid, methanesulfonic acid, trifluoroacetic acid or sulphuric acid. Preferably, the activating agent is trifluoromethanesulfonic anhydride ((CF₃SO₂)₂O, Tf₂O).

Typically, step b) is carried out at a temperature from −60° C. to −50° C.

The process can comprise, after step b), a step of purifying the compound of formula (I) obtained at the end of step b)-, for example by column chromatography.

When step b) leads to a photoinitiator of formula (I), wherein Y^{y-} is the desired anion, the process is free from step c). For example, when the activating agent is trifluoromethanesulfonic anhydride ((CF₃SO₂)₂O, Tf₂O), a photoinitiator of formula (I) is obtained wherein the anion Y^{y-} is is CF₃SO₃⁻. If CF₃SO₃ is the desired anion Y^{y-} in formula (I), then no step c) is performed.

When step b) leads to a photoinitiator of formula (I), wherein Y^{y-} is not the desired anion, the process comprises step c) of ion exchange. In the example above, if the desired anion Y^{y-} in formula (I) differs from CF₃SO₃⁻, for example if PF₆⁻ is the desired Y′^{y-}, then step c) is performed, typically with sodium hexafluorophosphate or hexafluorophosphoric acid.

At step c), the salt comprising Y^{y-} as anion can be alkaline metal salt, for example a sodium or potassium salt.

Step c) is typically carried out in the presence of an organic solvent. Suitable organic solvents include chloroform, dichloromethane and acetic acid.

Scheme 1 hereafter illustrates the process for the preparation of photoinitiators of formula (I).

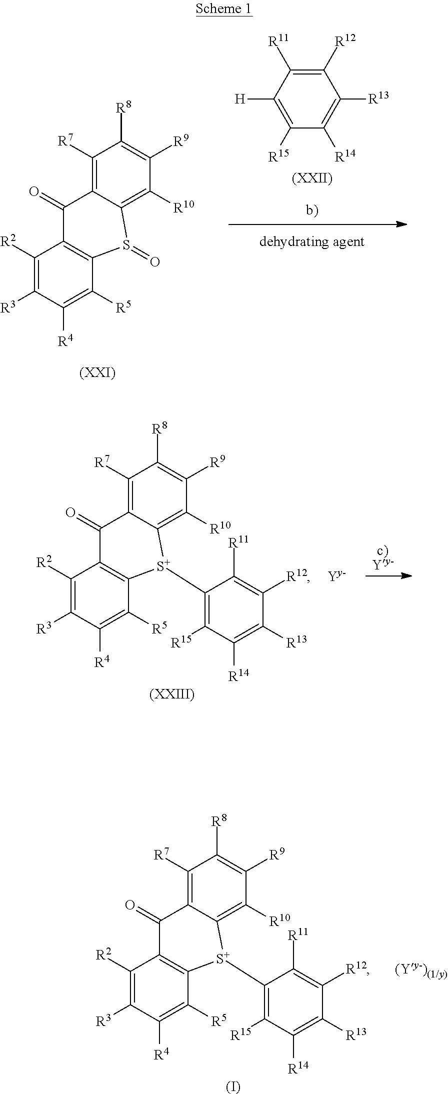

The process can comprise, prior to step b), a step a) of preparing the compound of formula (XXI) by oxidizing a compound of formula (XX):

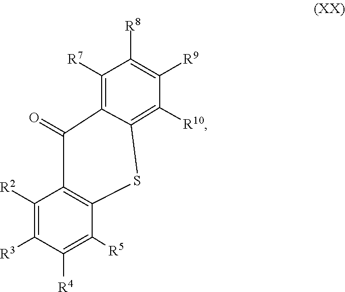

wherein R², R³, R⁴, R⁵, R⁷, R⁸, R⁹, R¹⁰ are as defined above. The oxidation is a selective oxidation of the compound of formula (XX) to the corresponding sulfoxide.

Step a) is generally carried out in the presence of an oxidizing agent, typically chosen from peroxy compounds (such as m-chloroperbenzoic acid (m-CPBA), peracetic acid, performic acid and hydrogen peroxide), transition metal salts (such as cerium ammonium nitrate) and hypervalent halogen compounds (such as sodium hypochlorite), the oxidizing agent being preferably m-CPBA.

Step a) may be carried out in the absence or presence of an organic solvent. Suitable organic solvents include chloroform, dichloromethane, acetonitrile or acetic acid.

Scheme 2 hereafter illustrate the process for the preparation of compounds of formula (XXI).

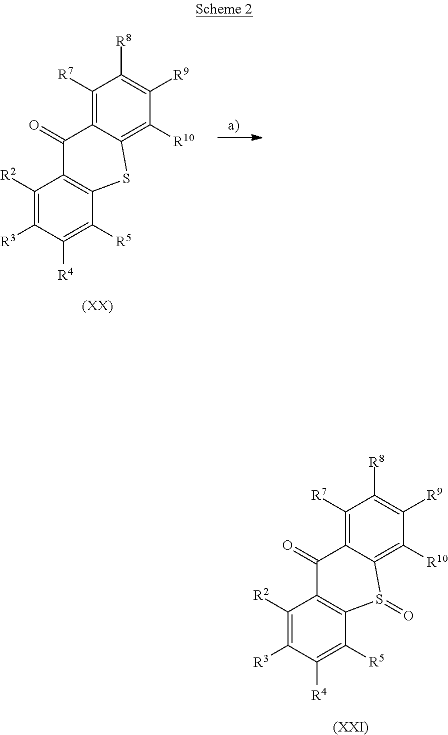

The process can comprise, after step a), of step of purifying the compound of formula (XXI), for example by column chromatography.

The photoinitiators of formula (V′) may be prepared by a method comprising the steps of:

q) adding acetic anhydride and an acid to a mixture of a compound of formula (XX′):

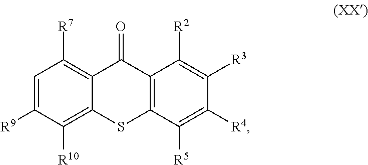

wherein R², R³, R⁴, R⁵, R⁷, R⁸, R⁹, R¹⁰ are as defined above,
and of a compound of formula (XXI′):

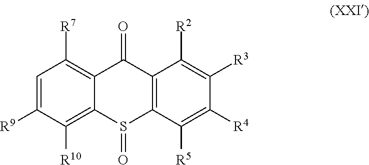

wherein R², R³, R⁴, R⁵, R¹, R⁸, R⁹, R¹⁰ are as defined above,
whereby a photoinitiator of formula (V′) is obtained,
r) when a photoinitiator of formula (V′) is desired, wherein Y^{y-} differs from the one obtained at step q), carrying out an ion exchange reaction with a salt comprising Y′^{y-} as anion, or an acid, the base of which is Y′^{y-}, to obtain a photoinitiator of formula (V) wherein Y′^{y-} has the same definition than Y^{y-} as defined above but differs from Y^{y-} obtained at step q).

The process for the preparation of a compound of formula (V′) is an object of the invention.

The process can comprise, prior to step q), a step p) of oxidizing a compound of formula (XX′):

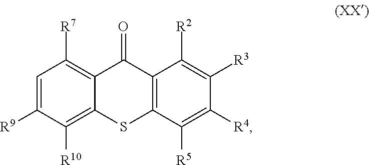

wherein R², R³, R⁴, R⁵, R⁷, R⁸, R⁹, R¹⁰ are as defined above, to form the compound of formula (XXI′):

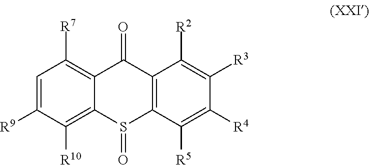

wherein R², R³, R⁴, R⁵, R⁷, R⁸, R⁹, R¹⁰ are as defined above.

Most preferably, the quantity of oxidizing agent used at step p) is adapted so that step p) leads to a mixture comprising both compound of formula (XXI′) and compound of formula (XX′). Said mixture can be used as the mixture of step p) without isolating the compounds of formula (XXI′) and (XX′).

The oxidation at step p) is preferably carried out with CH₃CO₃H as oxidizing agent, generally in an organic solvent such as dichloromethane. Typically, step p) is carried out at a temperature from 10° C. to 30° C. Step p) can comprise a sub-step of quenching, for example with an aqueous solution of Na₂SO₃.

Step q) is generally implemented in the presence of acetic anhydride and of a mineral acid, preferably sulfuric acid. Typically, step q) is carried out at a temperature starting from 0° C. to 5° C., then the reaction mixture is warmed to ambient temperature (about 20° C.). the process can comprise after step q) a step q′) of washing the reaction mixture obtained at the end of step q) with an aqueous solution, typically water.

When step q) leads to a photoinitiator of formula (V′), wherein Y^{y-} is the desired anion, the process is free from step r). For example, when the acid is H₂SO₄, a photoinitiator of formula (V′) is obtained wherein the anion Y^{y-} is is HSO₄⁻. If HSO₄⁻ is the desired anion Y^{y-} in formula (I), then no step r) is performed.

When step q) leads to a photoinitiator of formula (V′), wherein Y^{y-} is not the desired anion, the process comprises step r) of ion exchange. In the example above, if the desired anion Y^{y-} in formula (I) differs from HSO₄⁻, for example if PF₆⁻ is the desired Y′^{y-}, then step r) is performed, typically with sodium hexafluorophosphate or hexafluorophosphoric acid.

Scheme 3 hereafter illustrates the process for the preparation of photoinitiators of formula (V′).

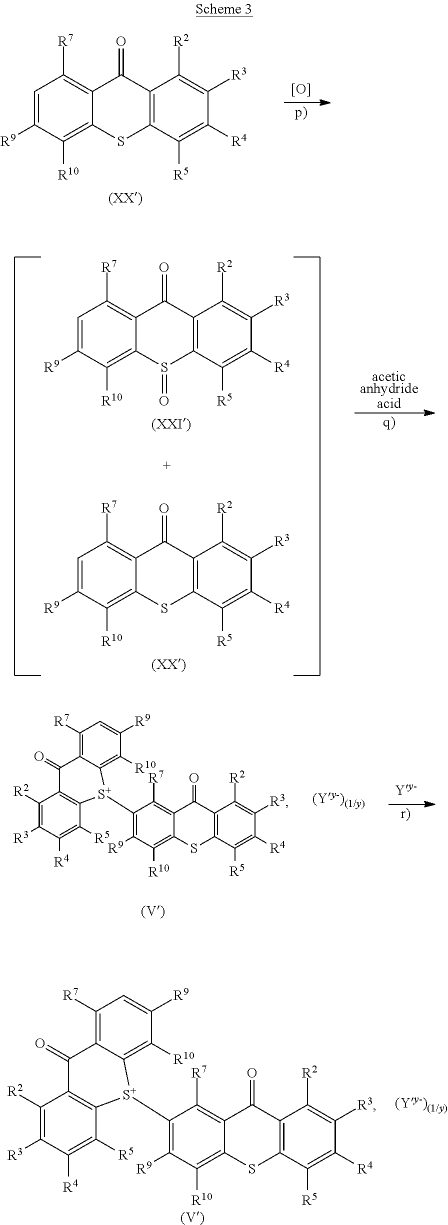

The composition used in the method comprises a cationically-polymerizable compound.

The composition comprises, in addition to the photoinitiator of formula (I), a cationically-polymerizable compound. The composition is thus a curable composition.

The composition may comprise a mixture of cationically-polymerizable compounds.

The term “cationically-polymerizable compound” means a compound comprising a polymerizing functional group which polymerizes via a cationic mechanism, for example a heterocyclic group or a carbon-carbon double bond substituted with an electrodonating group. In a cationic polymerization mechanism, a cationic initiator forms a Brønsted or Lewis acid species that binds to the cationically-polymerizable compound which then becomes reactive and leads to chain growth by reaction with another cationically-polymerizable compound.

The cationically-polymerizable compound may be selected from epoxy-functionalized compounds, oxetanes, oxolanes, cyclic acetals, cyclic lactones, thiiranes, thietanes, spiro orthoesters, ethylenically unsaturated compounds other than (meth)acrylates, derivatives thereof and mixtures thereof, and is preferably chosen among epoxy-functionalized compounds, oxetanes, polyols and mixtures thereof.

The curable composition may include from 5% to 99%, preferably from 10% to 98%, more preferably from 20% to 97%, by weight of the one or more cationically-polymerizable compound based on the total weight of the curable composition. If the composition comprises a mixture of cationically-polymerizable compounds, the above weight percentages may be calculated using the weight of the mixture of cationically-polymerizable compounds.

In a preferred embodiment, the cationically-polymerizable compound comprises at least one compound selected from epoxide, oxetane, oxolane, cyclic acetal, cyclic lactone, thiiranes, thiethanes, spiro orthoester, vinyl ether, and mixtures thereof.

In a most preferred embodiment, the cationically-polymerizable compound comprises a cycloaliphatic epoxide and optionally an oxetane.

### Epoxy Compound

The epoxy compound is also named epoxide or epoxy functional compound in the present invention.

The epoxy functional compounds may be monomers and/or oligomers.

Exemplary epoxy functional compounds suitable for use include mono-epoxides, di-epoxides, and poly-epoxides (compounds containing three or more epoxy groups per molecule. Alicyclic polyglycidyl compounds and cycloaliphatic polyepoxides are two classes of suitable epoxy functional compounds. Such compounds contain two or more epoxide groups per molecule and may have a cycloaliphatic ring structure that contains the epoxide groups as side groups (pendant to the cycloaliphatic ring) or may have a structure where the epoxide groups are part of an alicyclic ring structure.

The epoxy functional compound may comprise, consist of or consist essentially of at least one epoxy ether. As used herein, the term “epoxy ether” means a compound comprising at least two epoxy groups and at least one ether bond (the ether bond being distinct from the cyclic ether bond in the epoxy groups). In particular, the epoxy ether may comprise at least two epoxy groups and at least two ether bonds (the ether bonds being distinct from the cyclic ether bonds in the epoxy groups).

The epoxy functional compound may comprise, consist of or consist essentially of at least one glycidyl ether. As used herein, the term “glycidyl ether” means a compound comprising at least two glycidyl ether groups. As used herein, the term “glycidyl ether group” means a group of the following formula (A):

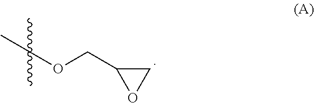 [SMILES: *OCC1CO1]

In one embodiment, the epoxy compound may comprise, consist of or consist essentially of at least one compound bearing two glycidyl ether groups, also referred to as a diglycidyl ether. In another embodiment, the epoxy may comprise, consist of or consist essentially of at least one compound bearing three glycidyl ether groups.

The epoxy may comprise, consist of or consist essentially of at least one compound selected from an aromatic epoxy, an aliphatic epoxy and mixtures thereof.

The epoxy may comprise, consist of or consist essentially of at least one aromatic epoxy. As used herein, the term an “aromatic epoxy” means a compound comprising at least two epoxy groups connected to one another by an aromatic linker.

As used herein, the term “aromatic linker” means a linker comprising at least one aromatic ring, preferably at least two aromatic rings, more preferably 2 or 3 aromatic rings. Araliphatic linkers, i.e. linkers comprising both an aromatic moiety and a non-aromatic moiety, are encompassed by the term aromatic linker.

The aromatic epoxy may be an aromatic glycidyl ether. As used herein, the term “aromatic glycidyl ether” means a compound comprising at least two glycidyl ether groups connected to one another by an aromatic linker. Such a compound may be represented by the following formula (B):

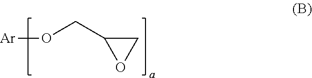

wherein Ar is an aromatic linker;
a is at least 2, preferably 2 to 10, more preferably 2 to 6.

The aromatic glycidyl ether may be a bisphenol-based glycidyl ether. As used herein, the term a “bisphenol-based glycidyl ether” means a compound comprising at least two glycidyl ether groups connected to one another by an aromatic linker containing a moiety derived from a bisphenol. Such a compound may be represented by the above formula (B) wherein a is 2 and Ar is represented by the following formula (C):

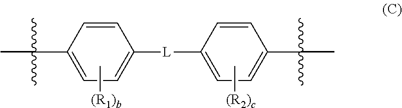

wherein L is a linker;
R₁ and R₂ are independently selected from alkyl, cycloalkyl, aryl and a halogen atom;
b and c are independently 0 to 4.

In particular, L may be a linker selected from bond, —CR₃R₄—, —C(═O)—, —SO—, —SO₂—, —C(═CCl₂)— and —CR₅R₆-Ph-CR₇R₈—; wherein

R₃ and R₄ are independently selected from H, alkyl, cycloalkyl, aryl, haloalkyl and perfluoroalkyl, or R₃ and R₄, with the carbon atoms to which they are attached, may form a ring;
R₅, R₆, R₇ and R₃ are independently selected from H, alkyl, cycloalkyl, aryl, haloalkyl and perfluoroalkyl;
Ph is phenylene optionally substituted with one or more groups selected from alkyl, cycloalkyl, aryl and a halogen atom.

More particularly, Ar may be the residue of a bisphenol without the OH groups. A compound according to formula (C) wherein Ar is the residue of a bisphenol without the OH groups may be referred to as a bisphenol-based epoxy ether, preferably a bisphenol-based glycidyl ether. Examples of suitable bisphenols are bisphenol A, bisphenol AP, bisphenol AF, bisphenol B, bisphenol BP, bisphenol C, bisphenol C2, bisphenol F, bisphenol G, bisphenol M, bisphenol S, bisphenol P, bisphenol PH, bisphenol TMC, bisphenol-Z, dinitrobisphenol A, tetrabromobisphenol A and combinations thereof.

The epoxy functional compound may comprise, consist of or consist essentially of at least one aliphatic epoxy. As used herein, the term an “aliphatic epoxy” means a compound comprising at least two epoxy groups connected to one another by an aliphatic linker.

As used herein, the term “aliphatic linker” means a linker that does not comprise any aromatic rings. It may be a linear or branched, cyclic or acyclic, saturated or unsaturated, hydrocarbon linker. It may be substituted by one or more groups, for example selected from hydroxyl, halogen (Br, Cl, I, F), carbonyl, amine, carboxylic acid, —C(═O)—OR′, —C(═O)—O—C(═O)—R′, each R′ being independently a C₁-C₆ alkyl. It may be interrupted by one or more bonds selected from ether-(—O—), ester (—C(═O)—O— or —O—C(═O)—), amide (—C(═O)—NH— or —NH—C(═O)—), urethane (—NH—C(═O)—O— or —O—C(═O)—NH—), urea (—NH—C(═O)—NH—), carbonate (—O—C(═O)—O—), and mixtures thereof.

The at least one aliphatic epoxy may be selected from an aliphatic glycidyl ether, an epoxidized vegetable oil and combinations thereof.

The aliphatic epoxy may be an aliphatic glycidyl ether. As used herein, the term “aliphatic glycidyl ether” means a compound comprising at least two glycidyl ether groups connected to one another by an aliphatic linker. Such a compound may be represented by the following formula (D):

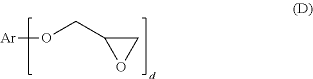

wherein Al is an aliphatic linker;
d is at least 2, preferably 2 to 10, more preferably 2 to 6.

In particular, Al may be an alkylene optionally interrupted by one or more ether or ester bonds or Al may correspond to a partially or fully hydrogenated derivative of the linker of formula (C).

More particularly, Al may be the residue of a polyol PO_{H} without the OH groups. Examples of suitable polyols PO_{H} include ethylene glycol, 1,2- or 1,3-propylene glycol, 1,2-, 1,3- or 1,4-butylene glycol, 1,5-pentanediol, 1,6-hexanediol, 1,8-octanediol, 1,9-nonanediol, 1,10-decanediol, 1,12-dodecanediol, 2-methyl-1,3-propanediol, 2,2-diethyl-1,3-propanediol, 3-methyl-1,5-pentanediol, 3,3-dimethyl-1,5-pentanediol, neopentyl glycol, 2,4-diethyl-1,5-pentanediol, cyclohexanediol, cyclohexane-1,4-dimethanol, norbornene dimethanol, norbornane dimethanol, tricyclodecanediol, tricyclodecane dimethanol, hydrogenated bisphenol A, B, F or S, trimethylolmethane, trimethylolethane, trimethylolpropane, di(trimethylolpropane), triethylolpropane, pentaerythritol, di(pentaerythritol), glycerol, di-, tri- or tetraglycerol, polyglycerol, di-, tri- or tetraethylene glycol, di-, tri- or tetrapropylene glycol, di-, tri- or tetrabutylene glycol, a polyethylene glycol, a polypropylene glycol, a polytetramethylene glycol, a poly(ethylene glycol-co-propylene glycol), a sugar alcohol (i.e. erythritol, threitol, arabitol, xylitol, ribitol, mannitol, sorbitol, glactitol, fucitol, iditol), a dianhydrohexitol (i.e. isosorbide, isomannide, isoidide), a hydroxylated vegetable oil, tris(2-hydroxyethyl)isocyanurate, a polybutadiene polyol, a polyester polyol, a polyether polyol, a polyorganosiloxane polyol, a polycarbonate polyol as well as the alkoxylated (e.g., ethoxylated and/or propoxylated) derivatives thereof and the derivatives obtained by ring-opening polymerization of E-caprolactone initiated with one of the aforementioned polyols. The epoxy compound may be an alkoxylated cycloaliphatic epoxide according to the following formula (E):

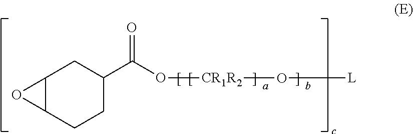

wherein
each R₁ and R₂ is independently selected from H and Me;
L is the residue of a polyol, preferably (HO—CH₂—)₃C-CH₂)₂O;
each a is independently from 2 to 4, preferably 2 or 4;
each b is independently 0 to 20 with the proviso that at least one b is not 0; c is at least 3, preferably from 3 to 10, in particular from 3 to 8, more particularly from 4 to 6.

The aliphatic epoxy compound may be an epoxidized vegetable oil.

As used herein the term “epoxidized vegetable oil” means an unsaturated vegetable oil wherein at least part of the carbon-carbon double bonds have been converted into epoxides. An unsaturated vegetable oil typically comprises one or more unsaturated diglycerides and/or triglycerides. Unsaturated diglycerides and triglycerides may correspond to diesters and triesters of glycerol with one or more fatty acids wherein at least part of the fatty acids are unsaturated fatty acids. Fatty acids may be defined as monocarboxylic acids comprising 4 to 32 carbon atoms, in particular 8 to 30 carbon atoms, more particularly 10 to 28 carbon atoms. Unsaturated fatty acids correspond to fatty acids containing one or more carbon-carbon double bonds. Examples of unsaturated fatty acids are myristoleic acid, palmitoleic acid, sapienic acid, oleic acid, ricinoleic acid, elaidic acid, vaccenic acid, linoleic acid, linoelaidic acid, a-linolenic acid, arachidonic acid, eicosapentaenoic acid, erucic acid, docosahexaenoic acid and combinations thereof. Unsaturated vegetable oils may be extracted from a plant or tree, for example from seeds, fruits, flowers, bark, wood, stems or leaves of a plant or tree. Examples of suitable epoxidized vegetable oil include epoxidized soybean oil, epoxidized linseed oil, epoxidized castor oil, epoxidized corn oil, epoxidized cottonseed oil, epoxidized perilla oil, epoxidized safflower oil, epoxidized palm oil, epoxidized coconut oil, epoxidized rapeseed oil, epoxidized jatropha oil, epoxidized rubber seed oil, epoxidized tung oil, epoxidized tall oil, and combinations thereof.

Also suitable are linear or branched epoxidized polyenes, such as epoxidized polybutadienes and copolymers thereof, polyisoprenes, and copolymers thereof, for example.

Examples of compounds in which the epoxide groups form part of an alicyclic ring system include bis(2,3-epoxycyclopentyl)ether; 2,3-epoxycyclopentyl glycidyl ether, 1,2-bis(2,3-epoxycyclopentyloxy)ethane; bis(4-hydroxycyclohexyl)methane diglycidyl ether, 2,2-bis(4-hydroxycyclohexyl)propane diglycidyl ether; 3,4-epoxycyclohexylmethyl-3′,4′-epoxycyclohexanecarboxylate; 3,4-epoxy-6-methyl-cyclohexylmethyl 3,4-epoxy-6-methylcyclohexanecarboxylate; di(3,4-epoxycyclohexylmethyl)hexanedioate; di(3,4-epoxy-6-methylcyclohexylmethyl)hexanedioate; ethylenebis(3,4-epoxycyclohexane-carboxylate, ethanediol di(3,4-epoxycyclohexylmethyl)ether; vinylcyclohexene dioxide; dicyclopentadiene diepoxide; and 2-(3,4-epoxycyclohexyl-5,5-spiro-3,4-epoxy-)cyclohexane-1,3-dioxane.

Suitable illustrative mono-epoxides include: glycidyl (meth)acrylate and (3,4-epoxycyclohexyl)methyl(meth)acrylate as well as other mono-epoxide compounds containing an epoxy group and a (meth)acrylate group.

Suitable illustrative di-epoxides include diglycidyl ethers of dialcohols and diglycidyl esters of di-acids such as: ethylene glycol diglycidyl ether, oligo- and polyethylene glycol diglycidyl ethers, propylene glycol diglycidyl ether, oligo- and polypropylene glycol diglycidyl ethers, butanediol diglycidyl ether, alkoxylated (e.g., ethoxylated, propoxylated) butanedioldiglycidyl ethers, neopentyl glycol diglycidyl ether, alkoxylated (e.g., ethoxylated, propoxylated) neopentyl glycol diglycidyl ethers, hexanediol diglycidyl ether, alkoxylated (e.g., ethoxylated, propoxylated) hexanediol diglycidyl ethers, cyclohexanedimethanol diglycidyl ether, alkoxylated (e.g., ethoxylated, propoxylated) cyclohexanedimethanol diglycidyl ethers, hydrogenated or nonhydrogenated bisphenol A diglycidyl ethers (BADGE), hydrogenated or nonhydrogenated bisphenol F diglycidyl ethers (BFDGE), diglycidyl ethers of alkoxylated (e.g., ethoxylated, propoxylated) bisphenols (such as bisphenol A or bisphenol F or hydrogenated derivatives thereof), diglycidyl esters of ortho-, iso- or terephthalic acid, diglycidyl esters of tetrahydrophthalic acid, and diglycidyl esters of hexahydrophthalic acid.

Suitable illustrative poly-epoxides include glycidyl ethers of compounds having three or more hydroxyl groups, such as hexane-2,4,6-triol; glycerol; 1,1,1-trimethylol propane; bistrimethylol propane; pentaerythritol; sorbitol; and alkoxylated (e.g., ethoxylated, propoxylated) derivatives thereof, epoxy novolac resins, and the like.

The curable composition, in certain embodiments, may comprise one or more polymerizable, heterocyclic moiety-containing compounds that comprise (in addition to one or more epoxy groups) one or more polymerizable sites of ethylenic unsaturation, such as may be supplied by a (meth)acrylate group, a (meth)acrylamide group, a vinyl group, an allyl group or the like. Glycidyl methacrylate, and glycidyl acrylate are specific examples of such a polymerizable, heterocyclic moiety-containing compounds. In the calculation of the relative amounts of oxetane and epoxy in the cationically curable compounds in the composition, these compounds are considered to be epoxies. Examples of suitable epoxy (meth)acrylates include the reaction products of acrylic or methacrylic acid or mixtures thereof with glycidyl ethers or esters.

### Oxetane Compound

The oxetane compound is also named oxetane or oxetane functional compound in the present invention.

The oxetanes may be monomers and/or oligomers.

Suitable illustrative oxetanes include oxetane itself and substituted derivatives thereof, provided the substituents do not interfere with the desired reaction/polymerization/curing of the oxetane. The substituent(s) may be, for example, alkyl groups, hydroxyalkyl groups, halo, haloalkyl groups, aryl groups, aralkyl groups and the like. The oxetane may be a mono-oxetane (a compound containing a single oxetane ring), a di-oxetane (a compound containing two oxetane rings), a tri-oxetane (a compound containing three oxetane rings), or an oxetane compound containing four or more oxetane rings. Examples of suitable oxetanes include, but are not limited to, oxetane, 3-ethyl-3-hydroxymethyl oxetane, 1,4-bis[(3-ethyl-3-oxetanylmethoxy)methyl]benzene, 3-ethyl-3-phenoxymethyl oxetane, 3-ethyl-3-{[(3-ethyloxetan-3-yl)methoxy]methyl}oxetane, 3,3-bis (chloromethyl oxetane), 3-ethyl-3-[(phenylmethoxy)methyl]-oxetane, 4,4′-bis(3-ethyl-3-oxetanyl)methoxymethyl]biphenyl, 3,3-bis(iodomethyl) oxetane, 3,3-bis(methoxymethyl) oxetane, 3,3-bis(phenoxymethyl) oxetane, 3-methyl-3-chloromethyl oxetane, 3,3-bis(acetoxymethyl) oxetane, 3,3-bis(fluoromethyl) oxetane, 3,3-bis(bromomethyl) oxetane, 3,3-dimethyl oxetane, 3-ethyl-3-[[(2-ethylhexyl)oxy]methyl]oxetane, bis[(3-ethyloxetan-3-yl)methoxy](dimethyl)silane, trimethylolpropane tris(3-ethyl-3-oxetanylmethyl)ether and the like and combinations thereof.

Examples of compounds having two or more oxetane rings in the compound, which may be used include: 3,7-bis(3-oxetanyl)-5-oxa-nonane, 3,3′-(1,3-(2-methylenyl)propanediylbis(oxymethylene))bis-(3-ethyloxetane), 1,4-bis[(3-ethyl-3-oxetanylmethoxy)methyl]benzene, 1,2-bis[(3-ethyl-3-oxetanylmethoxy)methyl]ethane, 1,3-bis[(3-ethyl-3-oxetanylmethoxy)methyl]propane, ethylene glycol bis(3-ethyl-3-oxetanylmethyl)ether, dicyclopentenyl bis(3-ethyl-3 oxetanylmethyl)ether, triethylene glycol bis(3-ethyl-3-oxetanylmethyl)ether, tetraethylene glycol bis(3-ethyl-3-oxetanylmethyl)ether, tricyclodecanediyldimethylene(3-ethyl-3-oxetanylmethyl)ether, trimethylolpropane tris(3-ethyl-3-oxetanylmethyl)ether, 1,4-bis(3-ethyl-3 oxetanylmethoxy)butane, 1,6-bis(3-ethyl-3-oxetanylmethoxy)hexane, pentaerythritol tris(3-ethyl-3-oxetanylmethyl)ether, pentaerythritol tetrakis(3-ethyl-3-oxetanylmethyl)ether, polyethylene glycol bis(3-ethyl-3-oxetanylmethyl)ether, dipentaerythritol hexakis(3-ethyl-3-oxetanylmethyl)ether, dipentaerythritol pentakis(3-ethyl-3-oxetanylmethyl)ether, dipentaerythritol tetrakis(3-ethyl-3-oxetanylmethyl)ether, caprolactone-modified dipentaerythritol hexakis(3-ethyl-3-oxetanylmethyl)ether, caprolactone-modified dipentaerythritol pentakis(3-ethyl-3-oxetanylmethyl)ether, ditrimethylolpropane tetrakis(3-ethyl-3-oxetanylmethyl)ether, EO-modified Bisphenol A bis(3-ethyl-3-oxetanylmethyl)ether, PO-modified Bisphenol A bis(3-ethyl-3-oxetanylmethyl)ether, EO-modified hydrogenated Bisphenol A bis(3-ethyl-3-oxetanylmethyl)ether, PO-modified hydrogenated Bisphenol A bis(3-ethyl-3-oxetanylmethyl)ether, EO-modified Bisphenol F (3-ethyl-3-oxetanylmethyl)ether, and the like and combinations thereof.

Additional examples of suitable oxetanes are described in the following patent documents, the disclosures of each of which are incorporated herein by reference in their entireties for all purposes: U.S. Pat. Publication No. 2010/0222512 A1, U.S. Pat. Nos. 3,835,003, 5,750,590, 5,674,922, 5,981,616, 6,469,108, 6,015,914, and 8,377,623. Suitable oxetanes are available from commercial sources, such as the oxetanes sold by the Toagosei Corporation under the tradenames OXT-221, OXT-121, OXT-101, OXT-212, OXT-211, CHOX, OX-SC, and PNOX-1009.

Also suitable are oxetanes that also include one or more polymerizable sites of ethylenic unsaturation, such as may be supplied by a (meth)acrylate group, a (meth)acrylamide group, a vinyl group, an allyl group or the like. 3-ethyl-3-(methacryloyloxy)methyloxetane or (3-ethyloxetane-3-yl) methyl acrylate are specific examples of such a compound. These compounds are included in the calculation of the amount of oxetane in the curable composition.

The curable composition may also include a compound containing two or more different types of polymerizable heterocyclic rings. For example, the compound may contain one or more oxetane rings and one or more epoxy rings (3-[(oxiranylmethoxy)methyl]oxetane is an example of such a compound). These compounds are included as both epoxy and oxetane containing compounds in the calculation of the relative amount of the oxetane based on the total amount of the oxetane and epoxy functional compounds in the composition.

### Other Cationically Curable Compounds

In addition to the oxetane functional compounds and the epoxy functional compounds, other cationically curable compounds may be included in the composition. Non-limiting examples of such compounds include compounds having free hydroxyl groups. The total weight of the cationically curable compounds, including epoxides, oxetanes and free hydroxyl components (such as hydroxyl groups from SpeedCure S130, OH from alcohol, polyol and OH from (meth)acrylates), should make 100% of the weight of the cationic system of the composition.

Polyols may therefore be optionally included in the curable composition. As used herein, the term “polymeric polyol” means a polymer bearing two or more primary, secondary or tertiary alcohol groups per molecule. As used herein, the term “nonpolymeric polyol” means a nonpolymeric compound bearing two or more hydroxyl groups per molecule. In the context of the present invention, the term “polymer” means a compound containing five or more repeating units per molecule and the term “nonpolymeric compound” means a compound containing up to four repeating units per molecule (and thus both monomeric compounds and oligomeric compounds containing 2 to 4 repeating units per molecule). For instance, ethylene glycol, diethylene glycol, triethylene glycol and tetraethylene glycol are all examples of nonpolymeric polyols, whereas polyethylene glycol containing five or more oxyalkylene repeating units is an example of a polymeric polyol.

Preferably, the hydroxyl groups are primary and/or secondary hydroxyl groups. In the case where the polyol is a polymeric polyol, the hydroxyl groups, according to certain embodiments, may be positioned at terminal ends of the polymer. However, it is also possible for hydroxyl groups to be present along the backbone of the polymer or on side chains or groups pendant to the polymer backbone. The polymer portion of the polymeric polyol may be comprised of a plurality of repeating units such as oxyalkylene units, ester units, carbonate units, acrylic units, alkylene units or the like or combinations thereof.

According to certain embodiments, the polymeric polyol may be represented by the following structure:

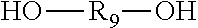

where R₉ is a polyether (e.g., polyoxyalkylene), polycarbonate, polydiene, polyorganosiloxane or polyester chain or linker.

Particularly preferred polymeric polyols include polyether diols and polyester diols. Suitable polyether diols include, for example, polytetramethylene glycols (hydroxyl-functionalized polymers of tetrahydrofuran) and polyethylene glycols (hydroxyl-functionalized polymers of ethylene oxide). Suitable polyester diols include, for example, poly(caprolactones), poly(lactides), poly(alkylene glycol adipates) and poly(alkylene glycol succinates).

Other types of polymeric polyols potentially useful in the present invention include polycarbonate polyols, polyorganosiloxane polyols (e.g., polydimethylsiloxane diols or polyols), and polydiene polyols (e.g., polybutadiene diols or polyol, including fully or partially hydrogenated polydiene polyols).

The molecular weight of the polymeric polyol may be varied as may be needed or desired in order to achieve particular properties in the cured composition obtained by curing the curable composition. For example, the number average molecular weight of the polymeric polyol may be at least 300, at least 350, or at least 400 g/mol. In other embodiments, the polymeric polyol may have a number average molecular weight of 5000 g/mol or less, 4500 g/mol or less, or 4000 g/mol or less. For example, the polymeric polyol may have a number average molecular weight of 250 to 5000 g/mol, 300 to 4500 g/mol or 350 to 4000 g/mol.

According to certain embodiments of the invention, the polyol may be represented by the following structure:

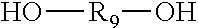

wherein R₉ is a divalent nonpolymeric aliphatic moiety optionally additionally comprising one or more heteroatoms (such as O, N, S and/or halogen).

In certain aspects of the invention, the diol is or includes a nonpolymeric polyol which is a hydrogenated dimer fatty acid (sometimes also referred to as a “dimer diol”), e.g., a diol obtained by dimerizing one or more unsaturated fatty acids such as oleic acid or linoleic acid and then hydrogenated to convert the carboxylic acid groups into hydroxyl groups. Pripol® 2033 (a product sold by Croda) is an example of a suitable commercially available hydrogenated dimer fatty acid.

Other types of suitable nonpolymeric polyols include, but are not limited to, C2-C12 aliphatic polyols, diols and oligomers thereof (containing up to four oxyalkylene repeating units). The aliphatic polyol or diol may be linear, branched or cyclic in structure, with the hydroxyl groups being both primary or both secondary or one or more of each type (one primary hydroxyl group and one secondary hydroxyl group, for example).

Examples of suitable C2-C12 aliphatic diols include, but are not limited to, ethylene glycol, 1,2-propylene glycol, 1,3-propanediol, 1,4-butanediol, 1,6-hexanediol, diethylene glycol, 2-methyl-1,3 propanediol, 3-methyl-1,5-pentanediol, 2,2-dimethyl-1,3-propanediol, 2,2,4-trimethyl 1,5-pentanediol, and 2-methyl-2-ethyl-1,3-propanediol, and oligomers thereof containing up to four oxyalkylene repeating units.

The optional at least one polyol, if present may be selected from ethylene glycol, propylene glycol, 1,3-propanediol, 1,2-, 1,3- or 1,4-butanediols, 2-methyl-1,3-propane diol (MPDiol), neopentyl glycol, alkoxylated derivatives of these, polyether diols, polyester diols, polycarbonate diols and combinations thereof.

The aliphatic diol (linear, branched or containing a ring structure) may be ethylene glycol, 1,2-propanedio, 1,3-propanediol, 1,4-butanediol, neopentyl glycol, 2-ethyl-1,3-hexanediol, 1,3-butanediol, 2-butyl-2-ethyl-1,3-propanediol, 2,4-diethyl-1,5-pentanediol and the like and short chain oligomers thereof (containing up to four oxyalkylene repeating units. Typically, the hydroxyl groups in such aliphatic diols are primary or secondary hydroxyl groups, which will react readily with the diisocyanates used to make the inherently reactive urethane acrylate oligomers.

The polyol may selected from ethylene glycol, propylene glycol, 1,3-propanediol, 1,2, 1,3 or 1,4 butanediols, 2-methyl-1,3-propane diol (MPDiol), neopentyl glycol, alkoxylated derivatives of these, polyether diols, polyester diols, or polysiloxane diols and combinations thereof.

The cationically curable compound may also be a cyclic ether compound, cyclic lactone compound, cyclic acetal compound, cyclic thioether compounds, spiro orthoester compounds or vinylether compound, for example.

### Hybrid Free-Radical/Cationic Compositions

The composition may be a hybrid free-radical/cationic composition, i.e. a composition that is cured by free radical polymerization and cationic polymerization.

The composition may thus further comprise a radically-polymerizable compound and optionally a radical photoinitiator.

Preferably, the radically-polymerizable compound comprises at least one ethylenically unsaturated compound, preferably a (meth)acrylate-functionalized compound.

As used herein, the term “(meth)acrylate-functionalized compound” means a monomer comprising a (meth)acrylate group, in particular an acrylate group. The term “(meth)acrylate-functionalized compound” here encompasses containing more than one (meth)acrylate group, such as 2, 3, 4, 5 or 6 (meth)acrylate groups, commonly referred to as “oligomers” comprising a (meth)acrylate group. The term “(meth)acrylate group” encompasses acrylate groups (—O—CO—CH═CH₂) and methacrylate groups (—O—CO—C(CH₃)═CH₂). Preferably, the (meth)acrylate-functionalized compound does not comprise any amino group. As used herein, the term “amino group” refers to a primary, secondary or tertiary amine group, but does not include any other type of nitrogen-containing group such as an amide, carbamate (urethane), urea, or sulfonamide group).

The (meth)acrylate-functionalized compound may have a molecular weight of less than 600 g/mol, in particular from 100 to 550 g/mol, more particularly 200 to 500 g/mol.

The curable composition may contain from 5% to 95%, preferably from 8% to 90%, more preferably from 10% to 80%, most preferably from 15 to 75% by weight of one or more ethylenically unsaturated compounds based on the total weight of the curable composition. If the composition comprises a mixture of ethylenically unsaturated compounds, the above weight percentage may be calculated using the weight of the mixture of ethylenically unsaturated compounds. In one embodiment, the curable composition may contain from 40% to 90%, from 45% to 85%, from 50% to 80%, or from 50% to 75% by weight of (meth)acrylate functional compounds based on the total weight of the curable composition. Alternatively, the curable composition may contain from 5% to 50%, from 10% to 45%, from 15% to 40% or from 15% to 30%, by weight of (meth)acrylate functional compounds based on the total weight of the curable composition.

Ethylenically unsaturated compounds suitable for use, other than the epoxy and oxetane containing compounds, include compounds containing at least one carbon-carbon double bond, in particular a carbon-carbon double bond capable of participating in a free radical reaction wherein at least one carbon of the carbon-carbon double bond becomes covalently bonded to an atom, in particular a carbon atom, in a second molecule. Such reactions may result in a polymerization or curing whereby the ethylenically unsaturated compound becomes part of a polymerized matrix or polymeric chain. In various embodiments of the invention, the additional ethylenically unsaturated compound(s) may contain one, two, three, four, five or more carbon-carbon double bonds per molecule. Combinations of multiple ethylenically unsaturated compounds containing different numbers of carbon-carbon double bonds may be utilized in the curable compositions. The carbon-carbon double bond may be present as part of an α,β-unsaturated carbonyl moiety, e.g., an α,β-unsaturated ester moiety such as an acrylate functional group or a methacrylate functional group or an α,β-unsaturated amide moiety such as an acrylamide functional group or a methacrylamide functional group. A carbon-carbon double bond may also be present in the additional ethylenically unsaturated compound in the form of a vinyl group —CH═CH₂ (such as an allyl group, —CH₂—CH═CH₂). Two or more different types of functional groups containing carbon-carbon double bonds may be present in the additional ethylenically unsaturated compound. For example, the ethylenically unsaturated compound may contain two or more functional groups selected from the group consisting of vinyl groups (including allyl groups), acrylate groups, methacrylate groups, acrylamide groups, methacrylamide groups and combinations thereof.

Ethylenically unsaturated compounds which are compounds suitable for use in the present invention include the following types of compounds (wherein “functional” refers to the number of (meth)acrylate functional groups per molecule, e.g., monofunctional=one (meth)acrylate group per molecule, difunctional=two (meth)acrylate groups per molecule):
i) cyclic monofunctional (meth)acrylate compounds, such as isobornyl (meth)acrylate, cyclohexyl (meth)acrylate, 4-tert-butyl cyclohexyl (meth)acrylate and alkoxylated analogues thereof;
ii) linear or branched monofunctional (meth)acrylate compounds, such as isodecyl (meth)acrylate, ethoxyethoxyethyl (meth)acrylate, polyethylene mono(meth)acrylates, neopentyl glycol mono(meth)acrylate and alkoxylated analogues thereof, as well as caprolactone-based mono(meth)acrylates prepared by addition of one, two, three or more moles of caprolactone to a hydroxyalkyl (meth)acrylate such as hydroxyethyl (meth)acrylate (“caprolactone adducts of hydroxyalkyl (meth)acrylates”);
iii) cyclic difunctional (meth)acrylate compounds, such as tricyclodecane dimethanol di(meth)acrylate, cyclohexane dimethanol di(meth)acrylate and alkoxylated analogues thereof;
iv) linear or branched difunctional (meth)acrylate compounds, such as polyethylene di(meth)acrylates, neopentyl glycol di(meth)acrylate and alkoxylated analogues thereof; and
v) trifunctional (meth)acrylate compounds, such as tris(2-hydroxyethyl) isocyanurate tri(meth)acrylate, trimethylolpropane tri(meth)acrylate and alkoxylated analogues thereof.

Illustrative examples of suitable ethylenically unsaturated compounds containing (meth)acrylate functionality include 1,2-, 1,3- or 1,4-butanediol di(meth)acrylate, 1,6-hexanediol di(meth)acrylate, alkoxylated 1,6-hexanediol di(meth)acrylate, alkoxylated aliphatic di(meth)acrylate, alkoxylated neopentyl glycol di(meth)acrylate, cyclohexane dimethanol di(meth)acrylate, diethylene glycol di(meth)acrylate, dipropylene glycol di(meth)acrylate, n-alkane (meth)acrylate, polyether di(meth)acrylates, ethoxylated bisphenol A di(meth)acrylate, ethylene glycol di(meth)acrylate, 1,2- or 1,3-propylene glycol di(meth)acrylate, neopentyl glycol di(meth)acrylate, polyester di(meth)acrylate, polyethylene glycol di(meth)acrylate, polypropylene glycol di(meth)acrylate, propoxylated neopentyl glycol diacrylate, tricyclodecane dimethanol di(meth)acrylate, triethylene glycol di(meth)acrylate, tetraethylene glycol di(meth)acrylate tripropylene glycol di(meth)acrylate, ditrimethylolpropane tetra(meth)acrylate, dipentaerythritol hexa(meth)acrylate, ethoxylated pentaerythritol tetra(meth)acrylate, dipentaerythritol penta(meth)acrylate, dipentaerythritol penta/hexa(meth)acrylate, penta(meth)acrylate ester, pentaerythritol tetra(meth)acrylate, ethoxylated trimethylolpropane tri(meth)acrylate, alkoxylated trimethylolpropane tri(meth)acrylate, propoxylated glyceryl tri(meth)acrylate, trimethylolpropane tri(meth)acrylate, pentaerythritol tri(meth)acrylate, propoxylated glyceryl tri(meth)acrylate, propoxylated trimethylolpropane tri(meth)acrylate, trimethylolpropane tri(meth)acrylate, tris (2-hydroxy ethyl) isocyanurate tri(meth)acrylate (also known as tris((meth)acryloxyethyl)isocyanurate), 2(2-ethoxyethoxy) ethyl (meth)acrylate, 2-phenoxyethyl (meth)acrylate, 3,3,5-trimethylcyclohexyl (meth)acrylate, alkoxylated lauryl (meth)acrylate, alkoxylated phenol (meth)acrylate, alkoxylated tetrahydrofurfuryl (meth)acrylate, caprolactone (meth)acrylate, (meth)acryloxyethyl di(caprolactone), cyclic trimethylolpropane formal (meth)acrylate, cycloaliphatic acrylate compound, dicyclopentadienyl (meth)acrylate, diethylene glycol methyl ether (meth)acrylate, ethoxylated (4) nonyl phenol (meth)acrylate, ethoxylated nonyl phenol (meth)acrylate, isobornyl (meth)acrylate, isodecyl (meth)acrylate, isooctyl (meth)acrylate, lauryl (meth)acrylate, methoxy polyethylene glycol (meth)acrylate, octyldecyl (meth)acrylate, stearyl (meth)acrylate, tetrahydrofurfuryl (meth)acrylate, tridecyl (meth)acrylate, and/or triethylene glycol ethyl ether (meth)acrylate, t-butyl cyclohexyl (meth)acrylate, alkyl (meth)acrylate, dicyclopentadiene di(meth)acrylate, alkoxylated nonylphenol (meth)acrylate, phenoxyethanol (meth)acrylate, octyl (meth)acrylate, decyl (meth)acrylate, dodecyl (meth)acrylate, tetradecyl (meth)acrylate, tridecyl (meth)acrylate, cetyl (meth)acrylate, hexadecyl (meth)acrylate, behenyl (meth)acrylate, diethylene glycol ethyl ether (meth)acrylate, diethylene glycol butyl ether (meth)acrylate, triethylene glycol methyl ether (meth)acrylate, 1,12-dodecanediol di(meth)acrylate, tricyclodecane methanol mono(meth)acrylate, glycerol carbonate (meth)acrylate and combinations thereof.

Suitable polyether (meth)acrylates include, but are not limited to, the condensation reaction products of acrylic or methacrylic acid or mixtures thereof with polyetherols which are polyether polyols. Suitable polyetherols can be linear or branched substances containing ether bonds and terminal hydroxyl groups. Polyetherols can be prepared by ring opening polymerization of cyclic ethers such as tetrahydrofuran or alkylene oxides with a starter molecule. Suitable starter molecules include water, hydroxyl functional materials, polyester polyols and amines.

One or more urethane diacrylates may be employed in certain embodiments. For example, the curable composition may comprise one or more urethane diacrylates comprising a difunctional aromatic urethane acrylate oligomer, a difunctional aliphatic urethane acrylate oligomer and combinations thereof. In certain embodiments, a difunctional aromatic urethane acrylate oligomer, such as that available from Sartomer USA, LLC (Exton, Pennsylvania) under the trade name CN9782, may be used as the one or more urethane diacrylates. In other embodiments, a difunctional aliphatic urethane acrylate oligomer, such as that available from Sartomer USA, LLC under the trade name CN9023, may be used as the one or more urethane diacrylates. CN9782, CN9023, CN978, CN965, CN9031, CN8881, and CN8886, all available from Sartomer USA, LLC, may all be advantageously employed as urethane diacrylates in the compositions.

Suitable acrylic (meth)acrylate oligomers (sometimes also referred to in the art as “acrylic oligomers”) include oligomers which may be described as substances having an oligomeric acrylic backbone which is functionalized with one or (meth)acrylate groups (which may be at a terminus of the oligomer or pendant to the acrylic backbone). The acrylic backbone may be a homopolymer, random copolymer or block copolymer comprised of repeating units of acrylic compounds. The acrylic compounds may be any (meth)acrylate such as C₁-C₆ alkyl (meth)acrylates as well as functionalized (meth)acrylates such as (meth)acrylates bearing hydroxyl, carboxylic acid and/or epoxy groups. Acrylic (meth)acrylate oligomers may be prepared using any procedures known in the art such as oligomerizing compounds, at least a portion of which are functionalized with hydroxyl, carboxylic acid and/or epoxy groups (e.g., hydroxyalkyl(meth)acrylates, (meth)acrylic acid, glycidyl (meth)acrylate) to obtain a functionalized oligomer intermediate, which is then reacted with one or more (meth)acrylate-containing reactants to introduce the desired (meth)acrylate functional groups. Suitable acrylic (meth)acrylate oligomers are commercially available from Sartomer USA, LLC under products designated as CN820, CN821, CN822 and CN823, for example.

Suitable free (meth)acrylate oligomers include, for example, polyester (meth)acrylates, epoxy (meth)acrylates, polyether (meth)acrylates, polyurethane (meth)acrylates, acrylic (meth)acrylate oligomers, epoxy-functional (meth)acrylate oligomers and combinations thereof.

According to certain embodiments, the curable composition is comprised of one or more ethylenically unsaturated compounds that contain one or more hydroxyl groups per molecule. Examples of such hydroxyl group-containing ethylenically unsaturated compounds include, but are not limited to, caprolactone adducts of hydroxyalkyl (meth)acrylates (compounds corresponding to the general formula H₂C═C(R)—C(═O)—O—R¹—(OC(═O)—[(CH₂)₅]ₙOH, wherein R═H, CH₃, R¹═C₂-C₄ alkylene, such as ethylene, propylene, butylene, and n=1-10, e.g., acryloxyethyl di(caprolactone)), hydroxyalkyl (meth)acrylates, alkoxylated (e.g., ethoxylated and/or propoxylated) hydroxyalkyl (meth)acrylates (including mono(meth)acrylates of ethylene glycol and propylene glycol oligomers and polymers), and the like.

In addition to the radically-polymerizable compound described above, the composition comprises in this embodiment a radical photoinitiator, in particular a radical photoinitiator having Norrish type I activity and/or Norrish type II activity, more particularly a radical photoinitiator having Norrish type I activity. The radical photoinitiator does not match formula (I).

Non-limiting types of radical photoinitiators suitable for use in the curable compositions include, for example, benzoins, benzoin ethers, acetophenones, a-hydroxy acetophenones, benzil, benzil ketals, phosphine oxides, acylphosphine oxides, a-hydroxyketones, phenylglyoxylates, a-aminoketones, benzoyl formates, acylgermanyl compounds, polymeric derivatives thereof, and mixtures thereof, but are not limited to, benzoin, benzoin methyl ether, benzoin ethyl ether, benzoin isopropyl ether, alpha-methylbenzoin, alpha-phenylbenzoin, Michler's ketone, 1-hydroxyphenyl ketones, acetophenone, 2,2-diethyloxyacetophenone, benzil, a-hydroxyketone, 2,4,6-trimethylbenzoyldiphenyl phosphine oxide, 2,2-dimethoxy-1,2-phenylacetophenone, 1-hydroxycyclohexyl phenyl ketone, 2-methyl-1-[4-(methylthio) phenyl]-2-morpholinopropanone, 2-hydroxy-2-methyl-1-phenyl-propanone, oligomeric a-hydroxy ketone, benzoyl phosphine oxides, phenylbis(2,4,6-trimethylbenzoyl)phosphine oxide, ethyl(2,4,6-trimethylbenzoyl)phenyl phosphinate, anisoin, benzoin isobutyl ether, 4-benzoylbiphenyl, 2-benzyl-2-(dimethylamino)-4′-morpholinobutyrophenone, 4,4′-dimethylbenzil, diphenyl(2,4,6-trimethylbenzoyl)phosphine oxide/2-hydroxy-2-methylpropiophenone 50/50 blend, 4′-ethoxyacetophenone, 2,4,6-trimethylbenzoyldiphenylphosphine oxide, 3′-hydroxyacetophenone, 4′-hydroxyacetophenone, methybenzoylformate, 4′-phenoxyacetophenone, polymeric derivatives thereof and combinations thereof.

Preferred radical photoinitiators are acetophenones, a-hydroxy acetophenones, phosphine oxides and acylphosphine oxides, more preferably acetophenones and acylphosphine oxides.

In particular, the radical photoinitiator may be selected from an acetophenone such as SpeedCure® BKL (2,2-dimethoxy-1,2-phenylacetophenone); an acylphosphine oxide such as Speed®Cure XKM (ethyl phenyl(2,4,6-trimethylbenzoyl)phosphinate), SpeedCure® BPO (phenyl bis(2,4,6-trimethylbenzoyl)-phosphine oxide), SpeedCure® TPO (2,4,6-trimethylbenzoyldiphenylphosphine oxide) or SpeedCure® TPO-L (ethyl (2,4,6-trimethylbenzoyl)phenyl phosphinate); and mixtures thereof.

The amount of radical photoinitiator may be varied as may be appropriate depending on the radical photoinitiator(s) selected, the amounts and types of polymerizable species present in the curable composition, the radiation source and the radiation conditions used, among other factors. Typically, however, the amount of radical photoinitiator may be from 0% to 10%, for example 0.05% to 10%, in particular 0.1% to 5%, more particularly 0.5 to 2%, by weight of radical photoinitiator based on the total weight of the curable composition. For example, the amount of radical photoinitiator may be from 0.01% to 5%, from 0.02% to 3%, from 0.05 to 2%, from 0.1 to 1.5% or from 0.2 to 1%, by weight based on the total weight of the curable composition. In another example, the amount of radical photoinitiator may be from 1% to 5%, from 1.5% to 5%, from 2 to 5%, from 2.5 to 5% or from 3 to 5%, by weight based on the total weight of the curable composition

### Fillers

The curable composition may include at least one filler, such as at least one opaque filler, which is insoluble in the other components of the light-curable composition. In particular, such filler does not dissolve in the curable composition. Further, it is preferred that the least one filler is insoluble in the solid resin matrix formed by curing the curable resin composition. The use of one or more fillers which are insoluble in the cured resin matrix makes possible the production of composite materials from the curable compositions of the present.

The filler or fillers may be of any suitable shape or form. For example, the filler may take the form of powder, beads, microspheres, particles, granules, wires, fibers or combinations thereof. If in particulate form, the particles may be spheroid, flat, irregular or elongated in shape. High aspect particulate fillers may be utilized, for example. Hollow as well as solid fillers are useful in the present invention. According to various embodiments of the invention, the filler may have an aspect ratio (i.e., the ratio of the length of an individual filler element, such as a particle or fiber, to the width of that individual filler element) of 1:1 or higher, e.g., greater than 1:1, at least 2:1, at least 3:1, at least 5:1, at least 10:1, at least 100:1, at least 1000:1; at least 10,000:1, at least 100,000:1, at least 500,000:1, at least 1,000,000:1 or even higher (i.e., effectively an infinite aspect ratio). According to other embodiments, the filler may have an aspect ratio not more than 2:1, not more than 3:1, not more than 5:1, not more than 10:1, not more than 100:1, not more than 1000:1; not more than 10,000:1, not more than 100,000:1, not more 500,000:1, or not more than 1,000,000:1

The surface of the filler may be modified in accordance with any of the methods or techniques known in the art. Such surface treatment methods include, without limitation, sizing (e.g., coating with one or more organic substances), silylation, oxidation, functionalization, neutralization, acidification, other chemical modifications and the like and combinations thereof.

The chemical nature of the filler may be varied and selected as may be desired in order to impart certain properties or characteristics to the product obtained upon curing the light-curable composition. For example, the filler may be inorganic or organic in character. Mixed organic/inorganic fillers may also be used. Carbon-based fillers (e.g., carbon fibers, carbon black, carbon nanotubes) as well as mineral fillers can be employed. One or more fibrous fillers (i.e., fillers in the form of fibers) may be utilized in especially preferred embodiments of the invention. Suitable exemplary fibrous fillers include carbon fibers (sometimes referred to as graphite fibers), glass fibers, silicon carbide fillers, boron fibers, alumina fibers, polymeric fibers (e.g., aramide fibers), metal fibers, natural fibers (such as fibers derived from plant sources) and combinations thereof. The fiber may be of natural or synthetic origin. Any of the following types of fiber can be used: short fibers (<10 mm in length), chopped fibers, long fibers (at least 10 mm in length), continuous fibers, woven continuous fibers, nonwoven continuous fibers, mats of woven fibers, mats of nonwoven fibers (e.g., random fiber mats), biaxial mats, unidirectional mats, continuous strands, unidirectional fibers, fiber tows, fiber fabrics, braided fibers, knitted fibers and the like and combinations thereof. Typically, suitable fibers will have a diameter of from about 2 to about 20 microns, e.g., from about 5 to about 10 microns. Hollow as well as solid fibers can be used; the fibers may be circular or irregular in cross-section.

Examples of other types of fillers which may be used in the curable compositions include clays (including organically modified clays and nanoclays), bentonite, silicates (e.g., magnesium silicates, talc, calcium silicates, wollastonite), metal oxides (e.g., zinc oxide, titanium dioxide, alumina), carbonates (e.g., calcium carbonate), mica, zeolites, talc, sulfates (e.g., calcium sulfate), and the like and combinations thereof.

In one embodiment, the curable composition comprises a relatively high loading of one or more fillers that are not opaque but which are capable of scattering rays of light to which the light-curable composition is exposed. For example, light scattering may occur where the refractive index of the filler is dissimilar to the refractive index of the portion of the curable composition which does not include the filler (which typically, prior to curing, is a liquid comprised of light-curable compounds, the photoinitiator system and possibly other non-filler additives). Such fillers may include, for example, glass fillers (e.g., glass fibers) and fillers comprised of transparent polymers. In such an embodiment, the curable composition may comprise at least 20%, at least 30% or at least 40% by weight, based on the total weight of the curable composition, of such light-scattering filler(s).

### Solvent

Advantageously, the curable compositions may be formulated to be solvent-free, i.e., free of any non-reactive volatile substances. However, in certain other embodiments of the invention, the curable composition may contain one or more solvents, in particular one or more organic solvents, which may be non-reactive organic solvents. In various embodiments, the solvent(s) may be relatively volatile, e.g., solvents having a boiling point at atmospheric pressure of not more than 150° C. In other embodiments, the solvent(s) may have a boiling point at atmospheric pressure of at least 40° C.

The solvent(s) may be selected so as to be capable of solubilizing one or more components of the curable composition and/or adjusting the viscosity or other rheological properties of the curable composition.

However, the curable compositions may alternatively be formulated so as to contain little or no non-reactive solvent, e.g., less than 10% or less than 5% or even 0% non-reactive solvent, based on the total weight of the curable composition. Such solvent-less or low-solvent compositions may be formulated using various components, including for example low viscosity reactive diluents, which are selected so as to render the curable composition sufficiently low in viscosity, even without solvent being present, that the curable composition can be easily applied at a suitable application temperature to a substrate surface so as to form a relatively thin, uniform layer.

Suitable solvents may include, for example, organic solvents such as: ketones; esters; carbonates; alcohols; aromatic solvents such as xylene, benzene, toluene, and ethylbenzene; alkanes; glycol ethers; ethers; amides; as well as combinations thereof.

In various embodiments of the invention, the curable compositions described herein are formulated to have a viscosity of less than 10,000 mPa·s (cP), or less than 5,000 mPa·s (cP), or less than 4,000 mPa·s (cP), or less than 3,000 mPa·s (cP), or less than 2,500 mPa·s (cP), or less than 2,000 mPa·s (cP), or less than 1,500 mPa·s (cP), or less than 1,000 mPa·s (cP) or even less than 500 mPa·s (cP) as measured at 25° C. using a Brookfield viscometer, model DV-II, using a 27 spindle (with the spindle speed varying typically between 20 and 200 rpm, depending on viscosity). In advantageous embodiments of the invention, the viscosity of the curable composition is from 200 to 1000 cPs at 25° C.

### Additives

The curable compositions may optionally contain one or more additives instead of or in addition to the above-mentioned ingredients. Such additives include, but are not limited to, free radical chain transfer agents, antioxidants, ultraviolet absorbers, light blockers, photostabilizers, foam inhibitors, flow or leveling agents, colorants, pigments, dispersants (wetting agents), slip additives, plasticizers, thixotropic agents, anti-yellowing agents, matting agents, impact modifiers, thermoplastics such as acrylic resins that do not contain any free radical-polymerizable functional groups, waxes or other various additives, including any of the additives conventionally utilized in the coating, sealant, adhesive, molding, 3D printing or ink arts.

In an embodiment, the curable composition comprises an anti-yellowing agent, preferably an anti-yellowing agent comprising an amino-group, such as an aminobenzoate group, or a thio group, such as an agent comprising both a (phenylthio) group and a carboxylic acid group, or mixtures thereof. Most preferably, the anti-yellowing agent is chosen from ethyl-4-(dimethylamino)benzoate, (phenylthio)acetic acid and mixtures thereof.

According to a second object, the invention relates to a curable composition comprising cationically-polymerizable compound and a photoinitiator of formula (I), preferably of formula (VIII) or (V′). All the embodiments described above for the cationically-polymerizable compound, for the photoinitiator of formula (I), and for the optional additional compounds apply.

According to a third object, the invention relates to a cured product obtained according to the method defined above. The cured product can be a 3D-printed article, coating, ink, adhesive, molding composition and sealant.

A most preferred application is the use of the method for the preparation of a 3D-printed article.

Accordingly, according to a fourth object, the invention relates to a method for the preparation of a 3D-printed article comprising the method of curing defined above.

Non-limiting examples of suitable 3D printing processes include stereolithography (SLA); digital light process (DLP); liquid crystal device (LCD); inkjet head (or multjet) printing; Continuous Liquid Interface Production (CLIP); extrusion type processes such as continuous fiber 3D printing and cast-in-motion 3D printing; and volumetric 3D printing. The building method may be “layer by layer” or continuous. The liquid may be in a vat, or deposited with an inkjet or gel deposition, for example.

When stereolithography is conducted above an oxygen-permeable build window, the production of an article using the curable composition may be enabled in a CLIP procedure by creating an oxygen-containing “dead zone” which is a thin uncured layer of the curable composition between the window and the surface of the cured article as it is being produced. In such a process, a curable composition is used in which curing (polymerization) is inhibited by the presence of molecular oxygen; such inhibition is typically observed, for example, in curable compositions which are capable of being cured by free radical mechanisms. The dead zone thickness which is desired may be maintained by selecting various control parameters such as photon flux and the optical and curing properties of the curable composition. The CLIP process proceeds by projecting a continuous sequence of actinic radiation (e.g., LED) images (which may be generated by a digital light-processing imaging unit, for example) through an oxygen-permeable, actinic radiation- (e.g., LED-) transparent window below a bath of the curable composition maintained in liquid form. A liquid interface below the advancing (growing) article is maintained by the dead zone created above the window. The curing article is continuously drawn out of the curable composition bath above the dead zone, which may be replenished by feeding into the bath additional quantities of the curable composition to compensate for the amounts of curable composition being cured and incorporated into the growing article.

In another embodiment, the curable composition will be supplied by ejecting it from a printhead rather than supplying it from a vat. This type of process is commonly referred to as inkjet or multijet 3D printing. One or more LED curing sources mounted just behind the inkjet printhead cures the curable composition immediately after it is applied to the build surface substrate or to previously applied layers. Two or more printheads can be used in the process which allows application of different compositions to different areas of each layer. For example, compositions of different colors or different physical properties can be simultaneously applied to create 3D printed parts of varying composition. In a common usage, support materials—which are later removed during post-processing—are deposited at the same time as the compositions used to create the desired 3D printed part. The printheads can operate at temperatures from about 25° C. up to about 100° C. Viscosities of the curable compositions are less than 30 mPa·s at the operating temperature of the printhead.

In an embodiment, the method for the preparation of a 3D-printed article comprises the following steps:
a) depositing a first layer of a composition as defined above onto a surface;
b) curing the first layer according to the method as defined above, at least partially, to provide a cured layer;
c) depositing a second layer of the composition onto the cured first layer;
d) curing the second layer according to the method as defined above, at least partially, to provide a cured second layer adhered to cured first layer; and
e) repeating steps c) and d) a desired number of times to build up the 3D-printed article.

Prior to curing, the composition may be applied to a substrate surface in any known conventional manner, for example, by spraying, knife coating, roller coating, casting, drum coating, dipping, jetting, extrusion, gel deposition, and the like and combinations thereof. Indirect application using a transfer process may also be used. A substrate may be any commercially relevant substrate, such as a high surface energy substrate or a low surface energy substrate, such as a metal substrate or plastic substrate, respectively. The substrates may comprise metal, paper, cardboard, glass, thermoplastics such as polyolefins, polycarbonate, acrylonitrile butadiene styrene (ABS), and blends thereof, composites, wood, leather and combinations thereof.

The process may comprise a further step f) comprising heating the three-dimensional article to a temperature effective to thermally cure the curable composition.

After the 3D article has been printed, it may be subjected to one or more post-processing steps. The post-processing steps can be selected from one or more of the following steps removal of any printed support structures, washing with water and/or organic solvents to remove residual resins, and post-curing using thermal treatment and/or actinic radiation either simultaneously or sequentially. The post-processing steps may be used to transform the freshly printed article into a finished, functional article ready to be used in its intended application.

In an embodiment, the method for the preparation of a 3D-printed article comprises the following steps:
a) providing a carrier and an optically transparent member having a build surface, the carrier and build surface defining a build region therebetween;
b) filling the build region with a composition as defined above;
c) continuously or intermittently curing part of the composition in the build region according to the method as defined above to form a cured composition; and
d) continuously or intermittently advancing the carrier away from the build surface to form the 3D-printed article from the cured composition.

The method may further comprise a post-curing step of heating or microwave irradiating the 3D printed article.

The post-processing steps described above can also be applied.

According to a fifth object, the invention related to a 3D printed article obtained with the method for the preparation of a 3D-printed article described above.

The examples and figures hereafter illustrate the invention.

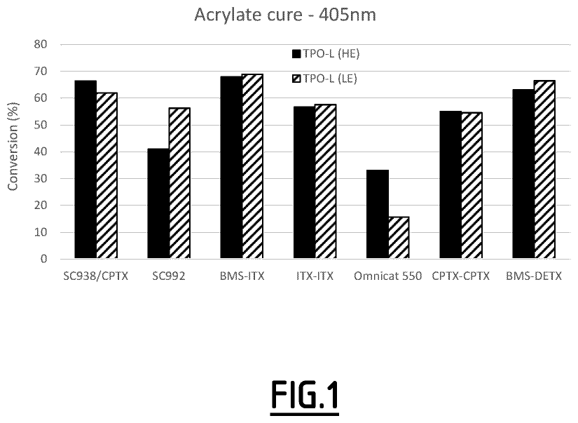

FIG. 1 provides the acrylate curing conversions at 405 nm for 0.5% Speedcure TPO-L in hybrid formulations.

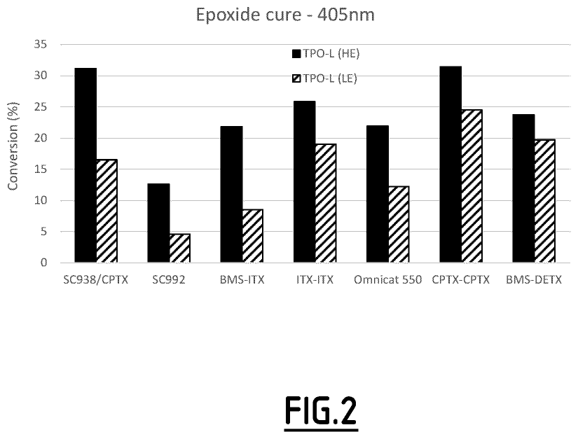

FIG. 2 provides the epoxide curing conversions at 405 nm for 0.5% Speedcure TPO-L in hybrid formulations.

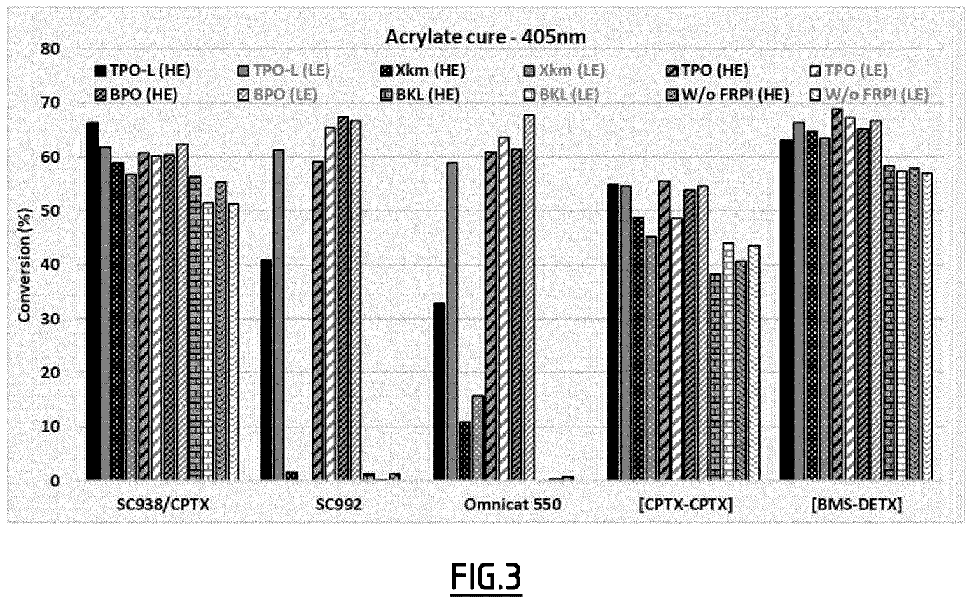

FIG. 3 provides the acrylate curing conversions at 405 nm with different radical photoinitiators or no radical photoinitiator in hybrid formulations.

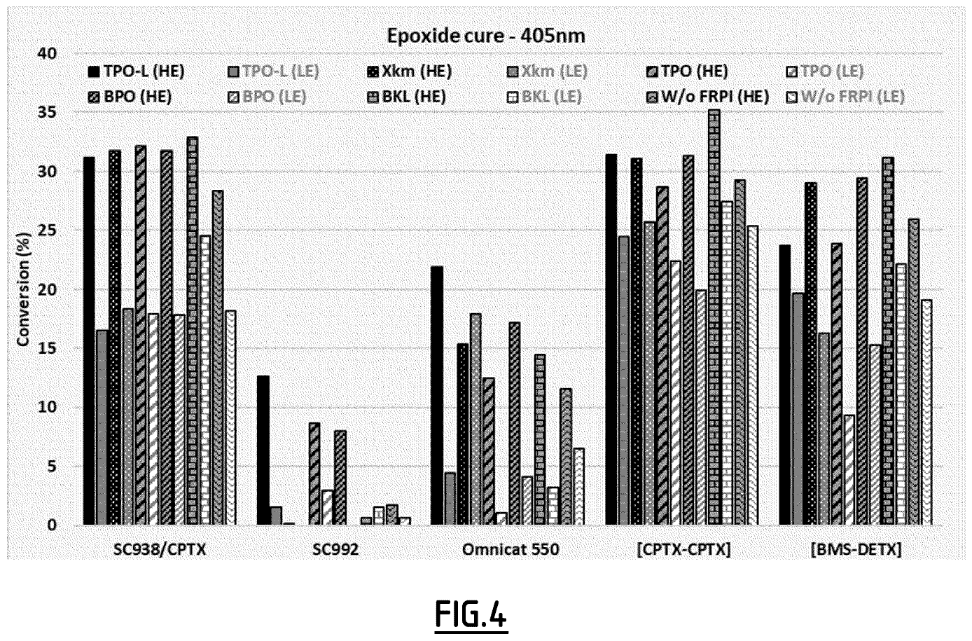

FIG. 4 provides the epoxide curing conversions at 405 nm with different radical photoinitiators or no radical photoinitiator in hybrid formulations.

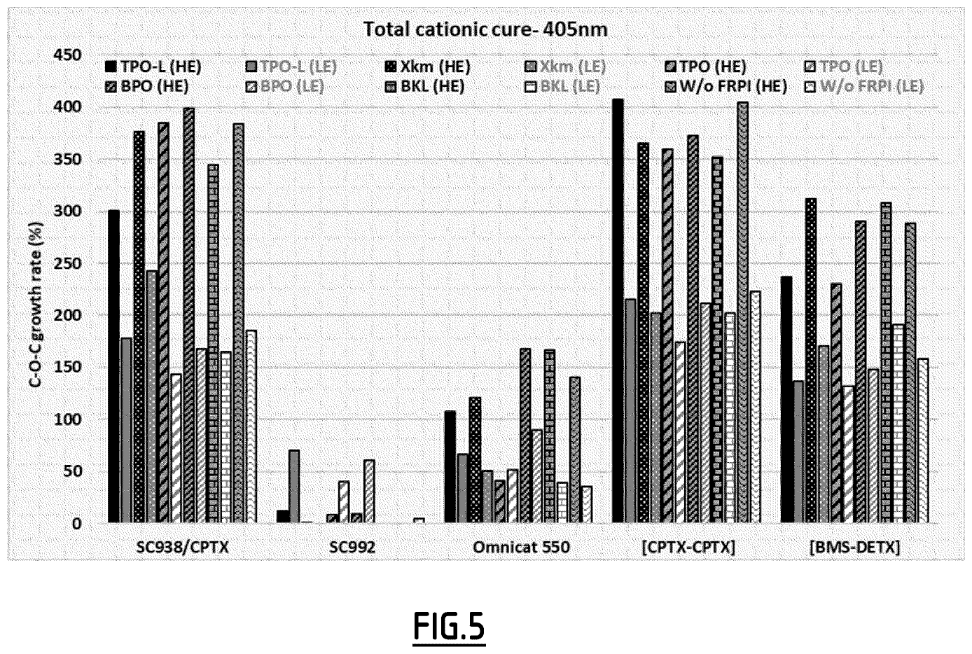

FIG. 5 provides the total cationic curing conversions at 405 nm with different radical photoinitiators or no radical photoinitiator in hybrid formulations.

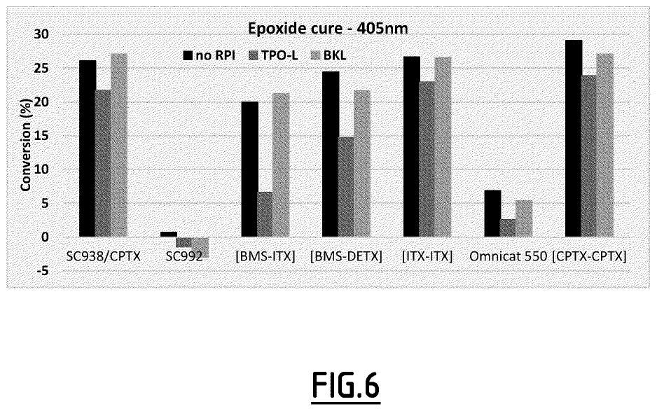

FIG. 6 provides the epoxide curing conversions at 405 nm in cationic formulations.

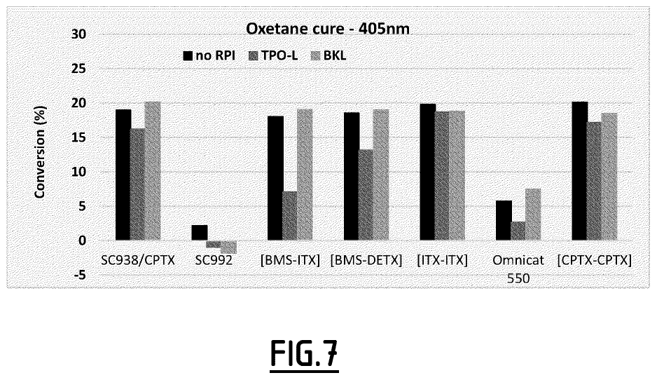

FIG. 7 provides the oxetane curing conversions at 405 nm in cationic formulations.

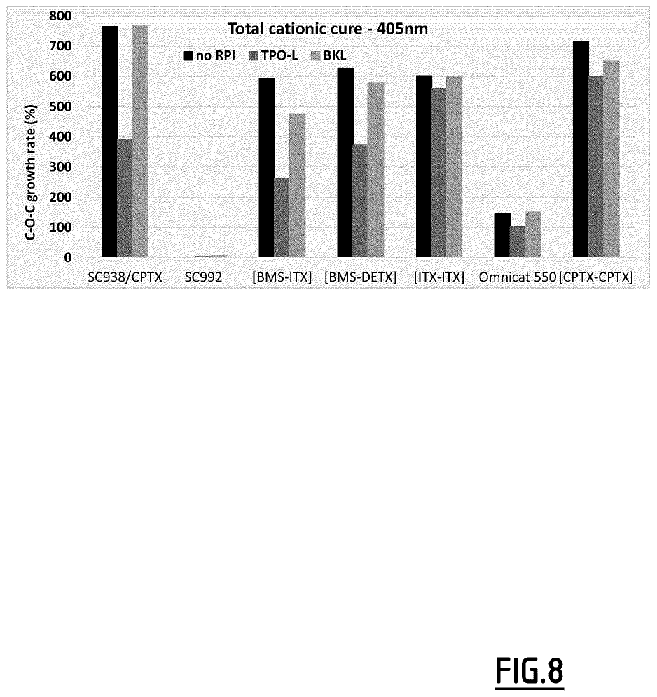

FIG. 8 provides the total cationic curing conversions in cationic formulations.

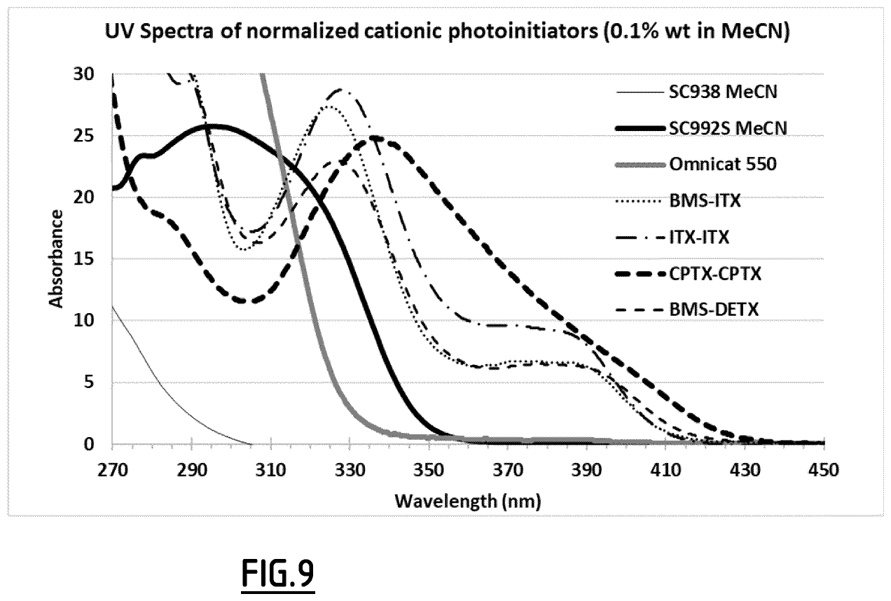

FIG. 9 provides the UV spectra of four new sulfonium salts, of Omnicat 550, Speedcure 992S (>99% active ingredient) and Speedcure 938

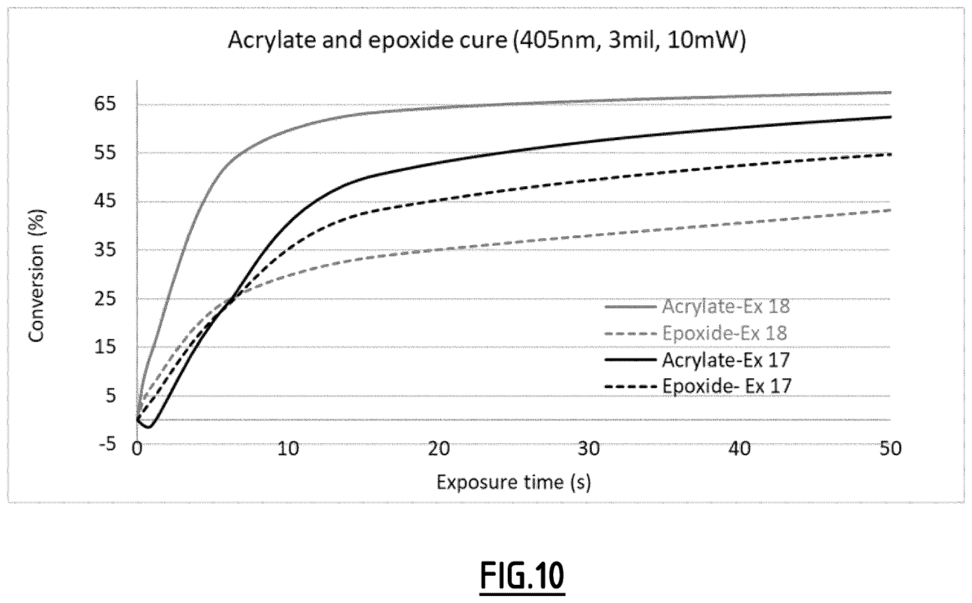

FIG. 10 provides the acrylate or epoxide curing conversions vs exposure time at at 10 mW of 405 nm LED

### EXAMPLE 1: PREPARATION OF PHOTOINITIATORS OF FORMULA (1)

### 1.1. Preparation of Intermediate Compounds of Formula (XXI) (Step a))

Compounds of formula (XXI) were prepared following the following general procedure 1.

To a solution of a diaryl sulfide of formula (XX) (1.64 mmol) in dichloromethane (10 mL) was slowly added m-CPBA (1.804 mmol) at 0° C. The mixture was stirred at 0° C. for 4 h and then gradually warmed to room temperature and stirred for 16 h. Saturated aq. sodium bicarbonate solution was added and the aqueous layer was then extracted with dichloromethane (3×3 mL). The combined organic layers were washed with brine, dried over magnesium sulfate, filtered and concentrated under reduced pressure. The obtained residue was purified by column chromatography on silica gel (PE/AcOEt) to afford the diaryl sulfoxide compound of formula (XXI).

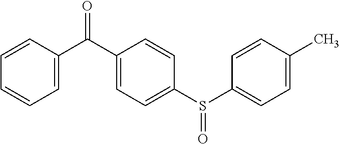 [SMILES: Cc1ccc(S(=O)c2ccc(C(=O)c3ccccc3)cc2)cc1]

### [4-(4-methylbenzene-1-sulfinyl)phenyl](phenyl)methanone

Prepared from {4-[(4-methylphenyl)sulfanyl]phenyl}(phenyl)methanone using General procedure 1. Yield 73%, white solid, m.p. 143-144° C.

¹H-NMR (400 MHz, CDCl₃): 7.86 (d, J=8.2 Hz, 2H), 7.78-7.74 (m, 4H), 7.60 (tt, J=7.3, 1.4 Hz, 1H), 7.58 (d, J=8.2 Hz, 2H), 7.50-7.46 (m, 2H), 7.29 (d, J=8.2 Hz, 2H), 2.38 (s, 3H).

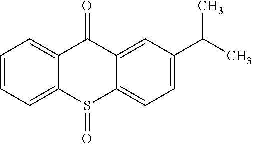 [SMILES: CC(C)c1ccc2c(c1)C(=O)c1ccccc1S2=O]

### 2-(propan-2-yl)-10λ⁴-thioxanthene-9,10-dione

Prepared from 2-(propan-2-yl)-9H-thioxanthen-9-one using General procedure 1. Yield 54%, pale yellow solid, m.p. 68-70° C.

¹H-NMR (400 MHz, CDCl₃): 8.37 (dd, J=7.8, 1.4 Hz, 1H), 8.24 (d, J=1.8 Hz, 1H), 8.16 (dd, J=8.0, 1.1 Hz, 1H), 8.09 (d, J=8.2 Hz, 1H), 7.85 (td, J=7.6, 1.4 Hz, 1H), 7.74-7.70 (m, 2H), 3.09 (septet, J=6.9 Hz, 1H), 1.33 (d, J=6.9 Hz, 6H).

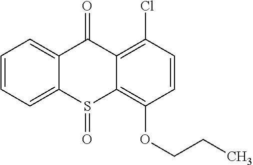 [SMILES: CCCOc1ccc(Cl)c2c1S(=O)c1ccccc1C2=O]

### 1-chloro-4-propoxy-10λ⁴-thioxanthene-9,10-dione

Prepared from 1-chloro-4-propoxy-9H-thioxanthen-9-one using General procedure 1. Yield 61%, yellow solid, m. p. 165-166° C.

¹H-NMR (400 MHz, CDCl₃): 8.20-8.15 (m, 1H), 7.96-7.91 (m, 1H), 7.77-7.70 (m, 2H), 7.62 (d, J=9.2 Hz, 1H), 7.18 (d, J=8.7 Hz, 1H), 4.21-4.10 (m, 2H), 2.02-1.93 (m, 2H), 1.15 (t, J=7.6 Hz, 3H).

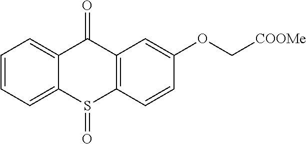 [SMILES: COC(=O)COc1ccc2c(c1)C(=O)c1ccccc1S2=O]

### Methyl [(9,10-dioxo-9,10-dihydro-10λ⁴-thioxanthen-2-yl)oxy]acetate

Prepared from methyl [(9-oxo-9H-thioxanthen-2-yl)oxy]acetate using General procedure 1. Yield 81%, light yellow solid.

¹H-NMR (400 MHz, CDCl₃): 8.35 (d, J=7.8 Hz, 1H), 8.14 (d, J=7.8 Hz, 1H), 8.07 (d, J=8.7 Hz, 1H), 7.85 (t, J=7.6 Hz, 1H), 7.78 (d, J=2.3 Hz, 1H), 7.71 (t, J=7.8 Hz, 1H), 7.42 (dd, J=8.7, 2.3 Hz, 1H), 4.80 (s, 2H), 3.82 (s, 3H).

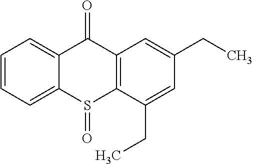 [SMILES: CCc1cc(CC)c2c(c1)C(=O)c1ccccc1S2=O]

### 2,4-diethyl-10λ⁴-thioxanthene-9,10-dione

Prepared from 2,4-diethyl-9H-thioxanthen-9-one using General procedure 1. Yield 62%, yellow solid, m.p. 99-100° C.

¹H-NMR (400 MHz, CDCl₃): 8.38 (dd, J=7.8, 1.4 Hz, 1H), 8.13 (d, J=1.8 Hz, 1H), 8.05 (dd, J=7.8, 1.4 Hz, 1H), 7.82 (td, J=7.3, 1.4 Hz, 1H), 7.74 (td, J=7.3, 1.4 Hz, 1H), 7.48 (d, J=1.8 Hz, 1H), 3.35-3.18 (m, 2H), 2.78 (q, J=7.8 Hz, 2H), 1.30 (t, J=7.6 Hz, 3H), 1.44 (t, J=7.6 Hz, 3H).

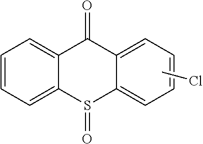

### Chloro-10λ⁴-thioxanthene-9,10-dione (mixture of 2- and 4-isomers)

Speedcure CTX (2.467 g; 10.0 mmol) was added in portions to trifluoroacetic acid (27 mL) with stirring. The obtained mixture was cooled to 0-5° C. and 35.6% aqueous hydrogen peroxide (1.003 g; 10.5 mmol) was added dropwise over 10 min. The mixture was then allowed to warm up to 20° C. over 4.5 h. Complete conversion of CTX starting material is confirmed by TLC (eluent petroleum ether/ethyl acetate 1:1).

The reaction mixture is added into 250 mL ice/water and stirred for 2 h. The solid product is filtered off and washed with water. The crude product is then collected, suspended in dichloromethane (50 mL) and the solution is evaporated to remove residual water. The crude product is purified by crystallization from petroleum ether/toluene. Yield 2.165 g (82%). ¹H-NMR indicates that the product is a mixture of 2- and 4-isomers (63:37).

1.2. Preparation of Photoinitiators of Formula (I) Wherein Y^{y-} is PF₆⁻ (Step b) and c))

### Step b):

Compounds of formula (I) were prepared following the following general procedure 2.

The appropriate aromatic sulfoxide of formula (XXI) (0.312 mmol) was dissolved in anhydrous dichloromethane (2.8 mL) and the resulting solution was cooled to between −60° C. and −50° C. Then trifluoromethanesulfonic anhydride (0.3432 mmol) used as activating agent was added to the solution and the mixture was stirred 20 min at a temperature between −60° C. and −50° C. The appropriate aromatic compound (0.312 mmol) of formula (XXII) was added and the mixture was gradually warmed to room temperature over a period of 15 h. The solvent was removed under reduce pressure and the residue was washed with diethyl ether (2×3 mL) to obtain the crude sulfonium trifluoromethanesulfonate salt intermediate of formula (I) wherein Y^{y-} is CF₃SO₃⁻.

Further purification was achieved by column chromatography on silica gel (eluting with dichloromethane/methanol).

### Step c):

In the examples were desired compounds of formula (I) wherein the anion Y^{y-} is PF₆⁻.

The solvent was removed in vacuo and the residue was dissolved in water (10 mL) at room temperature. A solution of sodium hexafluorophosphate (1.2 mol eq.) in water (1 mL) was added followed by chloroform (10 mL) and the mixture was stirred overnight at room temperature. The organic layer was separated and the aqueous phase was extracted with chloroform (2×5 mL). The solvent was evaporated to give the sulfonium hexafluorophosphate compound of formula (I) wherein Y^{y-} is PF₆⁻.

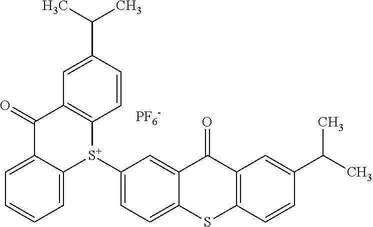

### 9-oxo-10-[9-oxo-7-(propan-2-yl)-9H-thioxanthen-2-yl]-2-(propan-2-yl)-9H-thioxanthen-10-ium hexafluorophosphate (1)

Prepared from 2-(propan-2-yl)-10λ⁴-thioxanthene-9,10-dione and 2-(propan-2-yl)-9H-thioxanthen-9-one using General procedure 2; Yield 28%; orange solid.

¹H-NMR (400 MHz, CDCl₃): 8.68-8.65 (m, 1H), 8.55-8.50 (m, 2H), 8.25-8.20 (m, 3H), 8.14 (d, J=8.2 Hz, 1H), 8.01-7.99 (m, 2H), 7.86-7.83 (m, 2H), 7.55 (dd, J=8.2, 1.8 Hz, 1H), 7.48 (d, J=8.2 Hz, 1H), 3.15 (septet, J=6.9 Hz, 1H), 2.99 (septet, J=6.9 Hz, 1H), 1.36-1.33 (m, 6H), 1.25 (d, J=6.9 Hz, 6H).

FT-IR (ATR; cm⁻¹): 505 (w), 531 (m), 556 (s), 631 (w), 640 (w), 688 (w), 713 (w), 741 (m), 752 (m), 782 (m), 834 (vs), 875 (w), 1061 (w), 1126 (w), 1206 (w), 1239 (w), 1265 (w), 1286 (w), 1300 (w), 1390 (w), 1416 (w), 1442 (w), 1472 (w), 1575 (w), 1590 (w), 1640 (w), 1671 (w), 2962 (w).

TOF MS ES+ m/z 507.1 Da (accurate mass 557.1444 Da).

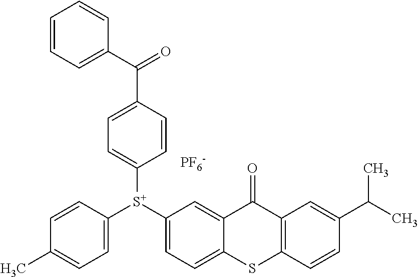

### (4-benzoylphenyl)(4-methylphenyl)[9-oxo-7-(propan-2-yl)-9H-thioxanthen-2-yl]sulfonium Hexafluorophosphate (2) (Comparative Example)

Prepared from [4-(4-methylbenzene-1-sulfinyl)phenyl](phenyl)methanone and 2-(propan-2-yl)-9H-thioxanthen-9-one using General procedure 2; Yield 39%; orange solid.

¹H-NMR (400 MHz, CDCl₃): 8.82 (d, J=2.3 Hz, 1H), 8.34 (d, J=2.3 Hz, 1H), 8.06-7.96 (m, 4H), 7.81-7.79 (m, 3H), 7.74-7.72 (m, 2H), 7.62-7.47 (m, 8H), 3.04 (septet, J=6.9 Hz, 1H), 2.48 (s, 3H), 1.30 (d, J=6.9 Hz, 6H).

FT-IR (ATR; cm⁻¹): 532 (m), 556 (s), 580 (w), 610 (w), 633 (w), 643 (w), 661 (w), 698 (w), 731 (w), 747 (w), 782 (m), 830 (vs), 876 (w), 926 (w), 1012 (w), 1061 (w), 1075 (w), 1126 (w), 1189 (w), 1205 (w), 1274 (m), 1310 (w), 1317 (w), 1397 (w), 1416 (w), 1448 (w), 1472 (w), 1579 (w), 1640 (w), 1660 (w), 2870 (vw), 2961 (vw).

TOF MS ES+ m/z 557.2 Da (accurate mass 557.1597 Da).

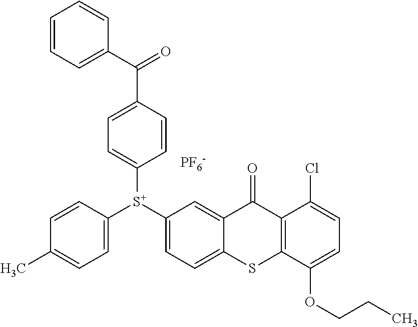

### (4-benzoylphenyl)(8-chloro-9-oxo-5-propoxy-9H-thioxanthen-2-yl)(4-methylphenyl)sulfonium hexafluorophosphate (3) (comparative example)

Prepared from [4-(4-methylbenzene-1-sulfinyl)phenyl](phenyl)methanone and 1-chloro-4-propoxy-9H-thioxanthen-9-one; Yield 54%; yellow solid.

¹H-NMR (400 MHz, CDCl₃): 8.64 (J=2.3 Hz, 1H), 8.07 (dd, J=8.7, 2.3 Hz, 1H), 8.02 (d, J=8.2 Hz, 2H), 7.94 (d, J=8.7 Hz, 1H), 7.81-7.79 (m, 4H), 7.72 (d, J=8.3 Hz, 2H), 7.60-7.55 (m, 3H), 7.51-7.48 (m, 2H), 7.42 (d, J=8.7 Hz, 1H), 7.05 (d, J=8.7 Hz, 1H), 4.10 (t, J=6.4 Hz, 2H), 2.49 (s, 3H), 1.91 (sextet, J=7.3 Hz, 2H), 1.11 (t, J=7.8 Hz, 3H).

FT-IR (ATR; cm⁻¹): 508 (w), 528 (w), 556 (s), 633 (w), 652 (w), 662 (m), 698 (m), 732 (w), 748 (w), 788 (m), 809 (s), 835 (vs), 876 (w), 926 (w), 958 (w), 1012 (w), 1063 (w), 1178 (w), 1189 (w), 1255 (m), 1275 (m), 1308 (w), 1397 (w), 1433 (w), 1448 (w), 1457 (w), 1546 (w), 1577 (w), 1653 (w), 2877 (w), 2967 (w), 3068 (w).

TOF MS ES+ m/z 607.1 Da (accurate mass 607.1163 Da).

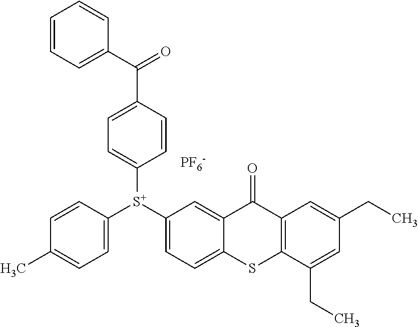

### (4-benzoylphenyl)(5,7-diethyl-9-oxo-9H-thioxanthen-2-yl)(4-methylphenyl)sulfonium Hexafluorophosphate (4) (Comparative Example)

Prepared from [4-(4-methylbenzene-1-sulfinyl)phenyl](phenyl)methanone and 2,4-diethyl-9H-thioxanthen-9-one using General procedure 2; Yield 41%; Orange solid.

¹H-NMR (400 MHz, CDCl₃): 8.81 (d, J=2.8 Hz, 1H), 8.20 (d, J=1.8 Hz, 1H), 8.03-8.01 (m, 3H), 7.82-7.79 (m, 3H), 7.75-7.73 (m, 2H), 7.61-7.55 (m, 4H), 7.50-7.43 (m, 4H), 2.86 (q, J=7.3 Hz, 2H), 2.75 (q, J=7.3 Hz, 2H), 2.48 (s, 3H), 1.35 (t, J=7.3 Hz, 3H), 1.28 (t, J=7.3 Hz, 3H).

FT-IR (ATR; cm⁻¹): 476 (w), 516 (w), 556 (s), 633 (w), 661 (m), 699 (m), 731 (m), 748 (w), 782 (m), 833 (vs), 876 (w), 926 (w), 1059 (w), 1190 (w), 1275 (w), 1310 (w), 1397 (w), 1426 (w), 1447 (w), 1579 (w), 1639 (w), 1660 (w), 2967 (vw).

TOF MS ES+ m/z 571.2 Da (accurate mass 571.1760 Da).

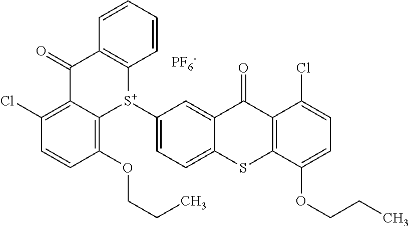

### 1-chloro-10-(8-chloro-9-oxo-5-propoxy-9H-thioxanthen-2-yl)-9-oxo-4-propoxy-9H-thioxanthen-10-ium Hexafluorophosphate (5)

Prepared from 1-chloro-4-propoxy-10λ⁴-thioxanthene-9,10-dione and 1-chloro-4-propoxy-9H-thioxanthen-9-one using General procedure 2; Yield 30%; yellow solid.

¹H-NMR (400 MHz, DMSO-d₆): 9.15 (d, J=2.3 Hz, 1H), 8.49-8.44 (m, 1H), 8.22-8.17 (m, 1H), 8.09 (d, J=9.2 Hz, 1H), 8.06 (d, J=9.2 Hz, 1H), 8.02-7.94 (m, 3H), 7.71 (d, J=9.2 Hz, 1H), 7.62 (d, J=9.2 Hz, 1H), 7.43 (d, J=8.7 Hz, 1H), 4.23-4.08 (m, 4H), 1.79 (sextet, J=7.3 Hz, 2H), 1.68 (sextet, J=6.9 Hz, 2H), 1.02 (t, J=7.8 Hz, 3H), 0.93 (t, J=7.3 Hz, 3H).

FT-IR (ATR; cm⁻¹): 495 (w), 534 (w), 546 (w), 557 (s), 644 (w), 651 (w), 687 (w), 694 (w), 718 (w), 742 (w), 761 (m), 773 (w), 799 (m), 808 (s), 820 (s), 836 (vs), 882 (w), 935 (w), 975 (w), 1058 (m), 1176 (w), 1238 m), 1254 (m), 1265 (m), 1276 (m), 1289 (w), 1303 (m), 1394 (w), 1435 (w), 1443 (w), 1549 (w), 1558 (w), 1571 (w), 1663 (w), 1683 (w), 2877 (w), 2959 (w), 3082 (w).

TOF MS ES+ m/z 607.1 Da (accurate mass 607.0566 Da).

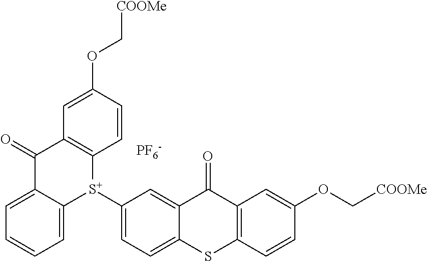

2-(2-methoxy-2-oxoethoxy)-10-[7-(2-methoxy-2-oxoethoxy)-9-oxo-9H-thioxanthen-2-yl]-9-oxo-9H-thioxanthen-10-ium Hexafluorophosphate (6)

Prepared from methyl [(9,10-dioxo-9,10-dihydro-10λ⁴-thioxanthen-2-yl)oxy]acetate and methyl [(9-oxo-9H-thioxanthen-2-yl)oxy]acetate using General procedure 2; Yield 34%; dark yellow solid.

¹H-NMR (400 MHz, acetone-d₆): 9.29 (bs, 1H), 8.71 (m, 1H), 8.41-8.35 (m, 2H), 8.16-8.09 (m, 2H), 8.04-7.95 (m, 3H), 7.80 (d, J=9.2 Hz, 1H), 7.75-7.68 (m, 2H), 7.51 (dd, J=8.7, 2.8 Hz, 1H), 5.12 (s, 2H), 4.97 (s, 2H), 3.784 (s, 3H), 3.777 (s, 3H).

FT-IR (ATR; cm⁻¹): 526 (m), 556 (s), 591 (w), 606 (w), 632 (w), 640 (w), 680 (w), 700 (w), 750 (m), 780 (m), 837 (vs), 1059 (m), 1082 (w), 1122 (w), 1167 (w), 1210 (m), 1300 (w), 1339 (w), 1421 (w), 1438 (w), 1475 (w), 1575 (w), 1589 (w), 1599 (w), 1634 (w), 1573 (w), 1748 (w).

TOF MS ES+ m/z 599.1 Da (accurate mass 599.0829 Da).

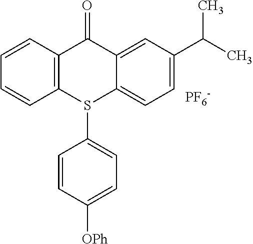

### 9-oxo-10-(4-phenoxyphenyl)-2-(propan-2-yl)-9H-thioxanthen-10-ium Hexafluorophosphate (12)

Prepared from 2-(propan-2-yl)-10λ⁴-thioxanthene-9,10-dione and diphenyl ether using General procedure 2; Yield 41%; light yellow semisolid.

Note: The product is a mixture of para- and ortho-isomers (approx. 4:1).

Major isomer: ¹H-NMR (300 MHz, CDCl₃): 8.65-8.62 (m, 1H), 8.48 (d, J=1.9 Hz, 1H), 8.14-8.10 (m, 1H), 8.05 (d, J=8.1 Hz, 1H), 7.99-7.95 (m, 2H), 7.83 (dd, J=8.7, 1.9 Hz, 1H), 7.68 (d, J=9.3 Hz, 2H), 7.44-7.38 (m, 2H), 7.28-7.20 (m, 1H), 7.07-7.00 (m, 4H), 3.15 (septet, J=6.8 Hz, 1H), 1.37-1.34 (m, 6H).

TOF MS ES+ m/z 423.1 Da (accurate mass 423.1413 Da).

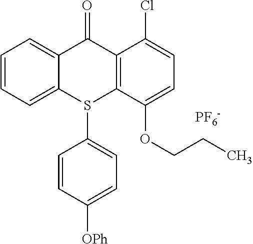

### 1-chloro-9-oxo-10-(4-phenoxyphenyl)-4-propoxy-9H-thioxanthen-10-ium hexafluorophosphate (16)

Prepared from 1-chloro-4-propoxy-10λ⁴-thioxanthene-9,10-dione and diphenyl ether using General procedure 2; Yield 30%; light yellow solid.

¹H-NMR (300 MHz, CDCl₃): 8.63-8.59 (m, 1H), 8.43-8.40 (m, 1H), 7.99-7.88 (m, 3H), 7.73 (d, J=8.7 Hz, 2H), 7.43-7.34 (m, 3H), 7.27-7.23 (m, 1H), 7.04-6.99 (m, 4H), 4.30-4.20 (m, 2H), 2.06-1.94 (m, 2H), 1.04 (t, J=7.5 Hz, 3H).

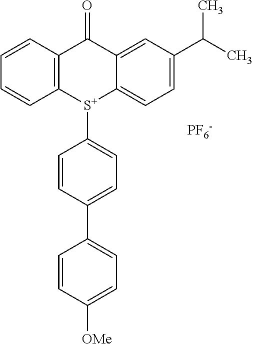

### 10-(4′-methoxy[1,1-biphenyl]-4-yl)-9-oxo-2-(propan-2-yl)-9H-thioxanthen-10-ium hexafluorophosphate (26)

Prepared from 2-(propan-2-yl)-10λ⁴-thioxanthene-9,10-dione and 4-methoxybiphenyl using General procedure 2; Yield 16%; yellow-brown semisolid.

¹H-NMR (300 MHz, CDCl₃): 8.66-8.61 (m, 1H), 8.48 (d, J=1.9 Hz, 1H), 8.35-8.28 (m, 1H), 8.23 (d, J=8.7 Hz, 1H), 7.99-7.93 (m, 2H), 7.88-7.80 (m, 3H), 7.70 (d, J=8.7 Hz, 2H), 7.45 (d, J=8.7 Hz, 2H), 6.95 (d, J=8.7 Hz, 2H), 3.83 (s, 3H), 3.14 (septet, J=6.9 Hz, 1H), 1.36-1.33 (m, 6H).

TOF MS ES+ m/z 437.2 Da (accurate mass 437.1568 Da).

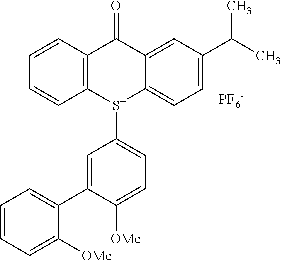

### 10-(2′,6-dimethoxy[1,1′-biphenyl]-3-yl)-9-oxo-2-(propan-2-yl)-9H-thioxanthen-10-ium hexafluorophosphate (28)

Prepared from 2-(propan-2-yl)-10λ⁴-thioxanthene-9,10-dione and 2,2′-dimethoxy-1,1′-biphenyl using General procedure 2; Yield 21%; light orange semisolid.

¹H-NMR (300 MHz, CDCl₃): 8.62-8.59 (m, 1H), 8.45 (d, J=1.9 Hz, 1H), 8.14 (dd, J=8.7, 2.5 Hz, 1H), 8.08-7.96 (m, 4H), 7.83 (dd, J=8.1, 1.9 Hz, 1H), 7.34-7.28 (m, 1H), 7.23-7.19 (m, 2H), 7.11 (dd, J=7.5, 1.9 Hz, 1H), 6.96-6.88 (m, 2H), 3.83 (s, 3H), 3.56 (s, 3H), 3.14 (septet, J=6.9 Hz, 1H), 1.34 (d, J=6.9 Hz, 6H).

TOF MS ES+ m/z 467.2 Da (accurate mass 467.1675 Da).

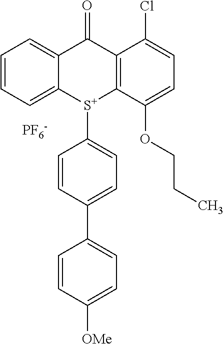

### 1-chloro-10-(4′-methoxy[1,1′-biphenyl]-4-yl)-9-oxo-4-propoxy-9H-thioxanthen-10-ium Hexafluorophosphate (32)

Prepared from 1-chloro-4-propoxy-10λ⁴-thioxanthene-9,10-dione and 4-methoxybiphenyl using General procedure 2; Yield 25%; light green semisolid.

¹H-NMR (300 MHz, CDCl₃): 8.46-8.43 (m, 1H), 8.33-8.30 (m, 1H), 7.99-7.91 (m, 3H), 7.71 (s, 4H), 7.46 (d, J=8.7 Hz, 2H), 7.42 (d, J=9.3 Hz, 1H), 6.96 (d, J=9.3 Hz, 2H), 4.28-4.21 (m, 2H), 3.84 (s, 3H), 2.03-1.91 (m, 2H), 1.02 (t, J=7.5 Hz, 3H).

TOF MS ES+ m/z 487.1 Da (accurate mass 487.1129 Da).

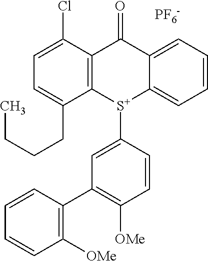

### 1-chloro-10-(2′,6-dimethoxy[1,1′-biphenyl]-3-yl)-9-oxo-4-propoxy-9H-thioxanthen-10-ium hexafluorophosphate (33)

Prepared from 1-chloro-4-propoxy-10λ⁴-thioxanthene-9,10-dione and 2,2′-dimethoxy-1,1′-biphenyl using General procedure 2; Yield 12%; dark brown semisolid.

¹H-NMR (300 MHz, CDCl₃): 8.42-8.39 (m, 1H), 8.23-8.20 (m, 1H), 7.98-7.89 (m, 4H), 7.45 (d, J=9.3 Hz, 1H), 7.37-7.31 (m, 1H), 7.27-7.26 (m, 1H), 7.17 (d, J=8.7 Hz, 1H), 7.12 (dd, J=7.5, 1.9 Hz, 1H), 6.98-6.91 (m, 2H), 4.25 (t, J=6.2 Hz, 2H), 3.82 (s, 3H), 3.61 (s, 3H), 2.00-1.88 (m, 2H), 1.02 (t, J=7.5 Hz, 3H).

TOF MS ES+ m/z 517.1 Da (accurate mass 517.1235 Da).

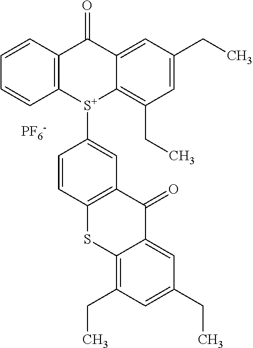

### 10-(5,7-diethyl-9-oxo-9H-thioxanthen-2-yl)-2,4-diethyl-9-oxo-9H-thioxanthen-10-ium Hexafluorophosphate (40)

Prepared from 2,4-diethyl-10λ⁴-thioxanthene-9,10-dione and 2,4-diethyl-9H-thioxanthen-9-one using General procedure 2; Yield 52%; yellow solid.

¹H-NMR (300 MHz, CDCl₃): 8.59-8.56 (m, 1H), 8.49-8.42 (m, 3H), 8.30 (d, J=1.9 Hz, 1H), 8.20 (d, J=1.9 Hz, 1H), 8.00-7.90 (m, 3H), 7.69 (d, J=1.9 Hz, 1H), 7.45-7.40 (m, 1H), 3.31-2.70 (m, 8H), 1.42-1.24 (m, 12H).

TOF MS ES+ m/z 535.2 Da (accurate mass 535.1760 Da).

### 1.3. Preparation of Photoinitiators of Formula (V′)

### General Procedure 3

To a solution of a diaryl sulfide of formula (XX′) in dichloromethane was slowly added CH₃CO₃H (35%). The mixture was stirred at 15-20° C. for 2 h and then quenched with an aqueous solution of Na₂SO₃. The mixture was not isolated, cooled to 0-4° C. and an excess of acetic anhydride and an excess of H₂SO₄ was added and the reaction mixture was then gradually warmed to room temperature, then washed with water.

In the examples were desired compounds of formula (V′) wherein the anion Y^{y-} is PF₆⁻.

A solution of sodium hexafluorophosphate (1.2 mol eq.) in water was added followed by MTBE and the mixture was stirred at room temperature. The precipitate was filtered and washed to give the sulfonium hexafluorophosphate compound of formula (V′) wherein Y^{y-} is PF₆⁻.

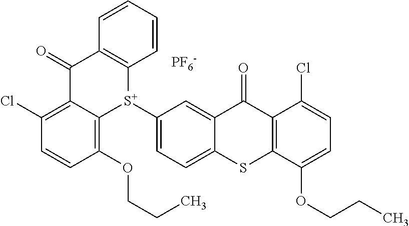

### 1-chloro-10-(8-chloro-9-oxo-5-propoxy-9H-thioxanthen-2-yl)-9-oxo-4-propoxy-9H-thioxanthen-10-ium hexafluorophosphate (5)

Prepared from 1-chloro-4-propoxy-9H-thioxanthen-9-one using General procedure 3; Yield 84%; yellow solid.

The analysis thereof is provided above.

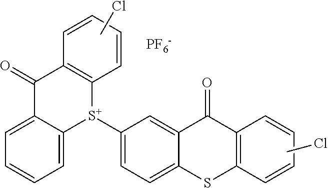

### Chloro-10-(chloro-9-oxo-9H-thioxanthen-2-yl)-9-oxo-9H-thioxanthen-10-ium Hexafluorophosphate (50) (Mixture of Isomers)

Speedcure CTX (1.2336 g; 5.0 mmol) and chloro-10λ⁴-thioxanthene-9,10-dione (mixture of 2- and 4-isomers) (1.3136 g; 5.0 mmol) were suspended in a mixture of acetic anhydride (6.0 mL) and dichloromethane (4.0 mL). The stirred mixture was cooled with ice-water and methanesulfonic acid (15.0 mL) was added dropwise over 5 minutes. The mixture is stirred with ice-water cooling for 2 h and then for 60 h at 20° C. with exclusion of light.

The dark red reaction mixture is added to a mixture of ice/water (150 g) and dichloromethane (125 mL) and stirred vigorously for 1 h at ambient temperature.

The mixture is phase separated and the organic phase is washed with water (50 mL), then separated and treated with a solution of potassium hexafluorophosphate (1.4726 g; 8.0 mmol) in water (20 mL). The biphasic mixture is stirred vigorously for 1.5 h, then partially evaporated to remove the dichloromethane. The yellow-orange suspension is then diluted with water (100 mL) and diisopropyl ether (75 mL) is added and the mixture is stirred vigorously for 2 h, then filtered. The collected solid is suspended in diisopropyl ether (50 mL) and water (15 mL) and stirred vigorously for 16 h. The solid product is filtered off, washed with diisopropyl ether (50 mL) and dried in vacuum. Yield 1.5247 g (48%); orange solid.

FT-IR (ATR; cm⁻¹): 1677 (w), 1639 (w), 1579 (w), 1453 (vw), 1439 (w), 1403 (w), 1395 (w), 1292 (m), 1264 (w), 1171 (w), 1143 (vw), 1099 (w), 1057 (w), 838 (vs), 818 (s), 799 (m), 780 (w), 759 (m), 749 (m), 739 (m), 671 (w), 556 (s), 526 (m), 511 (m).

TOF MS ES+ m/z 491.0 Da (accurate mass 490.9729 Da).

### Example 2: Curing Properties of the Photoinitiators of Example 1

### 2.1. Curing Performance at 365 and 385 nm

The curing performance of individual products prepared above was assessed using real time FT-IR measurement. The photoinitiators were dissolved in cycloaliphatic epoxy resin UViCure S105 (available from Sartomer) at the indicated wt % loading, applied onto the FT-IR measurement plate and irradiated with the indicated LED light source. The polymerization rate and final reactive group conversion were determined by monitoring the change of the relevant infrared band near 900 cm⁻¹ corresponding to an epoxide ring during irradiation. The molar extinction coefficients for the photoinitiator products (ε, expressed in M⁻¹ cm⁻¹) were determined in acetonitrile at 10-3 M or 10-5 M concentration.

TABLE 1
Curing performance at 365 and 385 nm of compounds assessed using real time FT-IR measurement

| | | LED@365 nm | | LED@385 nm | | | |
|---|---|---|---|---|---|---|---|
| | | UViCure S105 | | UViCure S105, | | | |
| | | 20 μm thickness | | 20 μm thickness | | | |
| | | under air | | under air | | | |
| | | LED @365 nm | | LED @385 nm | | | |
| | | (120 W/cm²) | | (160 W/cm²) | | | |
| | | Polymerization | | Polymerization | | ε (M⁻¹ | ε (M⁻¹ |
| | | rate | | rate | | cm⁻¹) | cm⁻¹) |
| | PI | Rp/[M₀]*100 | Final | Rp/[M₀]*100 | Final | at 365 | at 385 |
| Photoinitiator | loading | (s⁻¹) | conversion | (s⁻¹) | conversion | nm | nm |
| Omnicat BL 550 | 4 wt % | 2.81 | 25% | 2.51 | 26% | 1100 | 560 |
| Speedcure 938/ | 1 wt %/ | 5.75 | 38% | 5.65 | 41% | 18170 | 26621 |
| Speedcure CPTX | 0.5 wt % | | | | | | |
| Speedcure 992s | 1 wt % | 3.04 | 35% | 1.31 | 46% | | |
| 1 | 1 wt % | 8.93 | 35% | 8.89 | 36% | 23380 | 23514 |
| 2 (comp.) | 1 wt % | 4.11 | 41% | 5.96 | 41% | 14201 | 14921 |
| 3 (comp.) | 1 wt % | 2.70 | 38% | 2.46 | 37% | 17066 | 17083 |
| 4 (comp.) | 1 wt % | 5.74 | 25% | 4.80 | 25% | 13154 | 13649 |
| 5 | 1 wt % | 6.57 | 36% | 5.90 | 39% | 37326 | 23429 |
| 6 | 1 wt % | 0.72 | 35% | 2.01 | 36% | 18314 | 24656 |
| 12 | 1 wt % | 2.93 | 32% | 2.74 | 36% | 185 | 137 |
| 16 | 1 wt % | 2.88 | 40% | 3.71 | 38% | 20030 | 6830 |
| 26 | 1 wt % | 4.94 | 26% | 3.08 | 28% | 3600 | 1622 |
| 28 | 1 wt % | 2.29 | 34% | 5.78 | 36% | 426 | 268 |
| 32 | 1 wt % | 3.73 | 28% | 4.86 | 33% | 4507 | 2055 |
| 33 | 1 wt % | 1.78 | 30% | 3.66 | 31% | 4496 | 2199 |
| 40 | 1 wt % | 2.72 | 18% | 5.89 | 38% | 19117 | 15504 |

### 2.2. Curing Performance at 405 nm

The curing performance of individual products prepared above was assessed using real time FT-IR measurement. The photoinitiators were dissolved either in neat trimethylolpropane triacrylate (TMPTA; available as SR351 from Sartomer) or in a 1:1 (w/w) mixture of TMPTA and UViCure S105 as indicated, applied onto the FT-IR measurement plate, laminated to prevent oxygen inhibition, and irradiated with the indicated LED light source. For the epoxy component, the polymerization rate and final reactive group conversion were determined by monitoring the change of the relevant infrared band near 900 cm⁻¹ corresponding to an epoxide ring. For the acrylate component, the C═C stretch band near 1625 cm⁻¹ was used.

TABLE 2
Curing performance at 405 nm of compounds assessed using real time FT-IR measurement

| | | LED@405 nm, | | | | | |
|---|---|---|---|---|---|---|---|
| | | TMPTA, 20 μm | | | | | |
| | | thickness, | | | | | |
| | | laminated, | | LED@405 nm, UViCure S105/TMPTA (1:1), | | | |
| | | LED @405 nm | | 20 μm thickness, laminated, | | | |
| | | (110 W/cm²) | | LED @405 nm (110 mW/cm²) | | | |
| | | Acrylate | | Acrylate | | Epoxy | |
| | | Polymerization | | Polymerization | | Polymerization | |
| | | rate | Acrylate | rate | | rate | |
| | PI | Rp/[M₀]*100 | Final | Rp/[M₀]*100 | Final | Rp/[M₀]*100 | Final |
| Photoinitiator | loading | (s⁻¹) | conversion | (s⁻¹) | conversion | (s⁻¹) | conversion |
| Omnicat BL550 | 4 wt % | 1.15 | 35% | 3.27 | 70% | 1.02 | 29% |
| Speedcure 938/ | 1 wt %/ | 8.27 | 55% | 11.13 | 80% | 3.78 | 45% |
| Speedcure CPTX | 0.5 wt % | | | | | | |
| 1 | 1 wt % | 3.43 | 38% | 10.57 | 83% | 8.12 | 47% |
| 2 (comp.) | 1 wt % | 4.58 | 53% | 11.56 | 81% | 6.12 | 45% |
| 3 (comp.) | 1 wt % | 5.88 | 45% | 5.69 | 75% | 2.43 | 35% |
| 4 (comp.) | 1 wt % | 5.70 | 64% | 12.20 | 83% | 6.29 | 45% |
| 5 | 1 wt % | 2.01 | 51% | 6.03 | 70% | 3.87 | 44% |
| 40 | 1 wt % | 5.86 | 65% | 10.81 | 83% | 6.46 | 52% |

### 2.3. Belt Curing Performance at 365 nm and 395 nm

The photoinitiators were dissolved either in neat UViCure S105E resin or a hybrid acrylate/epoxy resin (prepared by mixing 60 wt parts UViCure S105E, 15 wt parts UViCure S130 and 25 wt parts SR492; all product are available from Sartomer). Formulations were prepared by combining all materials in the given proportions and then stirring at 30-40° C. until the samples were fully homogeneous; the formulations were then allowed to cool to room temperature. For all experiments, the formulations were cured on Leneta Form 3N-31 gloss finish paper at 6 μm and 24 μm film thickness using a belt-cure instrument; the films were prepared using a K-bar. All films were then cured under an LED lamp at a given belt speed. ‘Depth cure’ for each formulation was assessed using the ‘thumb-twist’ test (where no visible mark is made when a thumb is pressed down firmly onto the coating with a twisting motion), and from the belt speed; the calculated cure speed (in m/min) is given.

TABLE 3
Belt curing performance at 365 nm

| | | LED 365 nm; 100% intensity | |
|---|---|---|---|
| | | (=70.1 mJ/cm²) belt speed | |
| | | 40 m/min; 24 μm film, under air. | |
| | | Cure speed in m/min | |
| | PI | in UViCure | in hybrid acrylate/ |
| Photoinitiator | loading | S105E resin | epoxy resin |
| Omnicat 550 | 2 wt % | 5.0 | 5.7 |
| Speedcure 938/ | 2 wt %/ | 8.7 | 11.1 |
| Speedcure CPTX | 0.5 wt % | | |
| 1 | 2 wt % | 12.2 | 20.0 |
| 2 (comp.) | 2 wt % | 4.6 | 11.1 |
| 3 (comp.) | 2 wt % | 4.8 | 2.2 |
| 4 (comp.) | 2 wt % | 3.7 | 17.8 |
| 5 | 2 wt % | 13.3 | 40.0 |

TABLE 4
Belt curing performance at 395 nm

| | | LED 395 nm; 24 μm film, under air. | |
|---|---|---|---|
| | | Cure speed in m/min | |
| | | | 80 m/min |
| | | 40 m/min | 50% intensity |
| | | 100% intensity | (=36.9 mJ/cm²) |
| | PI | (=164.5 mJ/cm²) in | in hybrid acrylate/ |
| Photoinitiator | loading | UViCure S105E resin | epoxy resin |
| Omnicat 550 | 2 wt % | 8.0 | 10.5 |
| Speedcure 938/ | 2 wt %/ | 20.0 | 16.0 |
| Speedcure CPTX | 0.5 wt % | | |
| 1 | 2 wt % | 20.0 | 40.0 |
| 2 (comp.) | 2 wt % | 10.0 | 16.4 |
| 3 (comp.) | 2 wt % | 8.7 | no cure |
| 4 (comp.) | 2 wt % | 5.2 | 15.1 |
| 5 | 2 wt % | 13.3 | 80.0 |

As can be seen from these results, the sulfonium salt photoinitiators according to the invention are effective photoinitiators for epoxy, acrylic and hybrid resin formulations under LED lamp conditions.

In particular, photoinitiators 2 and 4 show higher cure speed than photoinitiators known from prior art such as Omnicat BL 550.

The belt curing results appear to show superior cure speed for photoinitiator 5 over photoinitiator 1 at 80 m/min in hybrid acrylate/epoxy resin at both 365 nm and 395 nm.

Photoinitiator 5 shows higher cure speed than photoinitiators known from prior art such as Omnicat BL 550.

Photoinitiators 26 and 32 show higher cure speed than photoinitiators known from prior art such as Omnicat BL 550.

### Example 3: Other Properties of the Photoinitiators of Example 1

### 3.1. Colour Measurements

Formulations comprising the photoinitiators and neat UViCure S105E resin were prepared as described at example 2.

TABLE 5
Colour index ‘b’ of formulations comprising
the compounds and irradiated at 365 or 385 nm

| | UViCure S105E, thin samples (20 | | UViCure S105E, thin samples (20 | |
|---|---|---|---|---|
| | μm), under air, LED @365 nm (120 | | μm), under air, LED @385 nm (160 | |
| | mW/cm²), 300 s irradiation | | mW/cm²), 300 s irradiation | |
| | Colour index ‘b’ | Colour index ‘b’ | Colour index ‘b’ | Colour index ‘b’ |
| | value Before | value After | value Before | value After |
| Photoinitiator | Polymerization | Polymerization | Polymerization | Polymerization |
| Omnicat BL550 | 4.94 | 5.24 | 5.54 | 5.50 |
| Speedcure 938/ | 6.64 | 13.23 | 6.60 | 8.26 |
| Speedcure CPTX | | | | |
| 1 | 6.38 | 8.81 | 6.13 | 8.71 |
| 2 (comp.) | 6.48 | 6.31 | 6.49 | 6.26 |
| 3 (comp.) | 11.28 | 7.40 | 8.40 | 7.48 |
| 4 (comp.) | 6.33 | 6.05 | 6.28 | 6.09 |
| 5 | 5.02 | 4.92 | 5.35 | 4.91 |

### 3.2. Solubility Data

Solubility of selected sulfonium salts in propylene carbonate at ambient temperature (20-25° C.) was determined.

TABLE 6
Solubility of compounds in propylene carbonate at 20-25° C.

| sample | Solubility in propylene carbonate | |
|---|---|---|
| Omnicat 550 | 25 | wt % |
| (comparative) | | |
| 1 | >50 | wt % |
| 2 (comp.) | >48 | wt % |
| 4 (comp.) | >47 | wt % |
| 5 | 42 | wt % |

### 3.3. Thermal Stability was Determined by DSC

Formulations comprising the photoinitiators and neat UViCure S105E resin and/or TMPTA resin were prepared as described at example 2.

TABLE 7
Thermal stability of formulations comprising
UViCure S105E resin and the compounds

| | | UViCure S105E resin, DSC at | |
|---|---|---|---|
| | | 10° C./min, onset temperature | |
| sample | loading | of thermal polymerisation | |
| Omnicat BL550 | 4 wt % | ~218° | C. |
| Speedcure 938/ | 1 wt %/ | 195° | C. |
| Speedcure CPTX | 0.5 wt % | | |
| 1 | 1 wt % | ~220° | C. |
| 2 (comp.) | 1 wt % | ~240° | C. |
| 5 | 1 wt % | ~214° | C. |
| Speedcure 992s | 1 wt % | >240° | C. |

TABLE 8
Thermal stability of formulations comprising TMPTA resin
or TMPTA/UViCure S105E resins and the compounds

| | | | TMPTA/UViCure S105E |
|---|---|---|---|
| | | TMPTA resin | (1:1) hybrid resin, |
| | | DSC at 10° C./min, onset | DSC at 10° C./min, onset |
| | | temperature of thermal | temperature of thermal |
| sample | loading | polymerisation | polymerisation |
| 1 | 1 wt % | 150° C. | 133° C. |
| 2 | 1 wt % | 170° C. | 117° C. |
| (comp.) | | | |
| 5 | 1 wt % | 165° C. | 120° C. |

### 3.4. Transmittance Data

The samples were prepared at 0.01% w/v in propylene carbonate.

TABLE 9
Transmittance of samples comprising the compounds at
0.01% w/v in propylene carbonate.
% Transmittance at 0.01% w/v in propylene carbonate

| | Omnicat 550 | 1 | 2 (comp.) | 4 (comp.) | 5 |
|---|---|---|---|---|---|
| 355 nm | 86.8 | 8.0 | 19.7 | 6.8 | 0.8 |
| 420 nm | 99.1 | 90.3 | 94.3 | 83.3 | 64.8 |

### 3.5. 6 Month Stability Study Vs References

TABLE 10
6 month stability study of formulations

| | | | Further |
|---|---|---|---|
| Sample (concentration) | Resin | Observation at 26 weeks | observations |
| Omnicat 550 (2 wt %) | UViCure S105 | Viscosity has increased, but | |
| | | solution remains free-flowing | |
| Omnicat 550 (2 wt %) | Hybrid resin* | Viscosity has increased, but | |
| | | solution remains free-flowing | |
| Speeedcure 938 (2 | UViCure S105 | Solution has gelled | Gelation occurred |
| wt %) + Speedcure | | | around week 15 |
| CPTX (0.5 wt %) | | | |
| Speeedcure 938 (2 | Hybrid resin* | Solution has gelled | Gelation occurred |
| wt %) + Speedcure | | | around week 15 |
| CPTX (0.5 wt %) | | | |
| 4 (2 wt %) (comp.) | UViCure S105 | Viscosity has increased, but | Gelation occurred |
| | | solution remains free-flowing | at week 30 |
| 4 (2 wt %) (comp.) | Hybrid resin* | Viscosity has increased, but | |
| | | solution remains free-flowing | |
| 5 (2 wt %) | UViCure S105 | Viscosity has increased, but | |
| | | solution remains free-flowing | |
| 5 (2 wt %) | Hybrid resin* | Highly viscous liquid at room | Crystallisation |
| | | temperature | occurred at week |
| | | | 18 (yellow needles) |

*Hybrid resin used had the following composition: UViCure S105E 60 wt %/UViCure S130 15 wt %/Sartomer SR492 25 wt %

### Example 4: Curing Performance in Cationic and Hybrid Formulations and 3D Printability in Hybrid System

### 4.1. Materials and Structures

TABLE 11
used materials and suppliers thereof

| Material | Composition | Supplier |
|---|---|---|
| SR 833S | 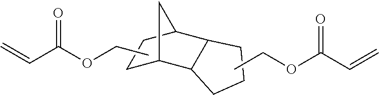 | Sartomer |
| | Tricyclodecane Dimethanol Diacrylate | |
| SR499 | 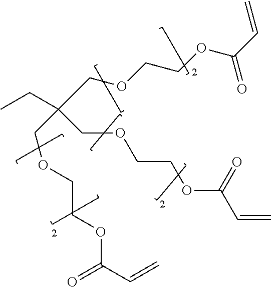 | Sartomer |
| | Ethoxylated (6) Trimethylolpropane Triacrylate | |
| SR259 | Polyethylene Glycol (200) Diarylate | Sartomer |
| UViCure S105 | 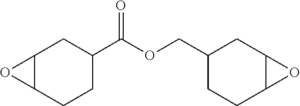 [SMILES: O=C(OCC1CCC2OC2C1)C1CCC2OC2C1] | Sartomer |
| | 3,4-Epoxycyclohexylmethyl 3,4- | |
| | epoxycyclohexanecarboxylate | |
| Eponex 1510 | 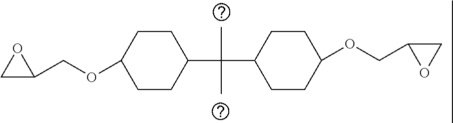 [SMILES: CC(C)(C1CCC(OCC2CO2)CC1)C1CCC(OCC2CO2)CC1] | Hexion |
| | Hydrogenated bisphenol A diglciyl ether | |
| UViCure S130 | 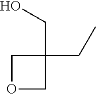 | Sartomer |
| | 3-Ethyl-3-hydroxymethyl-oxetane | |
| Speedcure TPO | 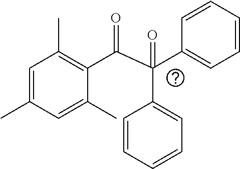 | Sartomer- Lambson |
| | 2,4,6-trimethylbenzoyldiphenylphosphine oxide | |
| Speedcure TPO-L | 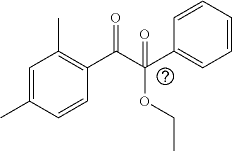 | Sartomer- Lambson |
| | Ethyl(2,4,6-trimethylbenzoyl)phenyl phosphinate | |
| Speedcure BPO | 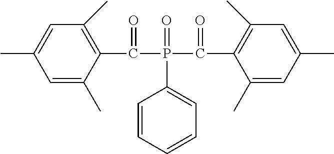 | Sartomer- Lambson |
| | Phenyl bis(2,4,6-trimethylbenzoyl)-phosphine oxide | |
| Speedcure XKm | 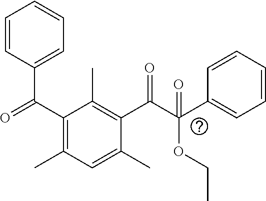 | Sartomer- Lambson |
| | Ethyl(3-benzoyl-2,4,6-trimethylbenzoyl) | |
| | (phenyl)phosphinate | |
| Speedcure BKL | 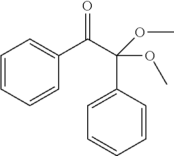 [SMILES: COC(OC)(C(=O)c1ccccc1)c1ccccc1] | Sartomer- Lambson |
| | 2,2-dimethoxy-1,2-diphenylethan-1-one | |
| Speedcure CPTX | 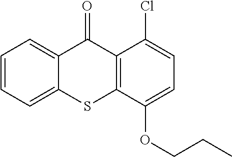 [SMILES: CCCOc1ccc(Cl)c2c(=O)c3ccccc3sc12] | Sartomer- Lambson |
| | 1-chloro-4-propoxythioxanthone | |
| Speedcure 938 | 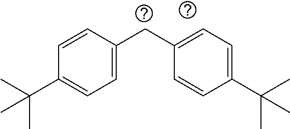 [SMILES: CC(C)(C)c1ccc(Cc2ccc(C(C)(C)C)cc2)cc1] | Sartomer- Lambson |
| | Bis (4-tert-butylphenyl) iodonium | |
| | hexafluorophosphate | |
| Speedcure 992 | 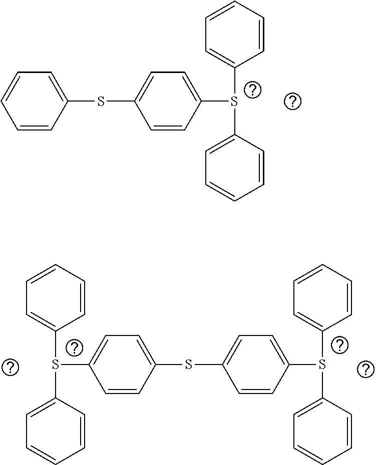 | Sartomer- Lambson |
| | 40% (4-{[4- | |
| | (diphenylsulfonylium)pheyl]sulfonayl}phenyl)diphenyl- | |
| | sulfonium bishexafluorophosphate in propylene | |
| | carbonate | |
| 1 | 9-oxo-10-[9-oxo-7-(propan-2-yl)-9H-thioxanthen-2- | prepared |
| | yl]-2-(propan-2-yl)-9H-thioxanthen-10-ium | according to |
| | hexafluorophosphate | example 1 |
| 2 (comp.) | (4-benzoylphenyl)(4-methylphenyl)[9-oxo-7-(propan- | prepared |
| | 2-yl)-9H-thioxanthen-2-yl]sulfonium | according to |
| | hexafluorophosphate | example 1 |
| 4 (comp.) | (4-benzoylphenyl)(5,7-diethyl-9-oxo-9H-thioxanthen- | prepared |
| | 2-yl)(4-methylphenyl)sulfonium hexafluorophosphate | according to |
| | | example 1 |
| 5 | 1-chloro-10-(8-chloro-9-oxo-5-propoxy-9H- | prepared |
| | thioxanthen-2-yl)-9-oxo-4-propoxy-9H-thioxanthen- | according to |
| | 10-ium hexafluorophosphate | example 1 |
| Omnicat 550 | 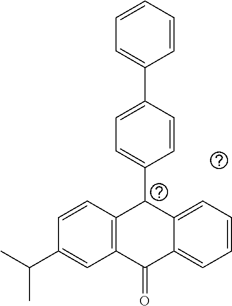 [SMILES: CC(C)c1ccc2c(c1)C(=O)c1ccccc1C2c1ccc(-c2ccccc2)cc1] | IGM |
| | 10-[1,1′-Biphenyl]-4-yl-2-(1-methylethyl)-9-oxo-9H- | |
| | thioxanthenium hexafluorophosphate | |
| Propylene carbonate | 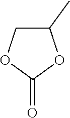 | Sigma-Aldrich |
| | 1,2-propanediol cyclic carbonate | |
| BYK-333 | Polyether-modified polydimethylsiloxane | BYK |

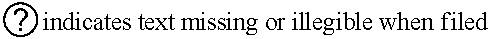

### 4.2 Sample Preparation and Test Methods

TABLE 12
Summary of matrixes

| | HEx | LEx | HEx | LEx | HEx | LEx | HEx | LEx | HEx | LEx | HEx | LEx | | CMx | CMx |
|---|---|---|---|---|---|---|---|---|---|---|---|---|---|---|---|
| Component | 1 | 1 | 2 | 2 | 3 | 3 | 4 | 4 | 5 | 5 | 6 | 6 | CMx | 1 | 2 |
| SR833S | 20.0 | 65.0 | 20.0 | 65.0 | 20.0 | 65.0 | 20.0 | 65.0 | 20.0 | 65.0 | 20.0 | 65.0 | | | |
| ECC S105 | 65.0 | 28.0 | 65.0 | 28.0 | 65.0 | 28.0 | 65.0 | 28.0 | 65.0 | 28.0 | 65.0 | 28.0 | 80.0 | 80.0 | 80.0 |
| Uvicure S130 | 15.0 | 7.0 | 15.0 | 7.0 | 15.0 | 7.0 | 15.0 | 7.0 | 15.0 | 7.0 | 15.0 | 7.0 | 20.0 | 20.0 | 20.0 |
| Speedcure | 0.5 | 0.5 | | | | | | | | | | | | 0.5 | |
| TPO-L | | | | | | | | | | | | | | | |
| Speedcure | | | 0.5 | 0.5 | | | | | | | | | | | |
| XKm | | | | | | | | | | | | | | | |
| Speedcure | | | | | 0.5 | 0.5 | | | | | | | | | |
| TPO | | | | | | | | | | | | | | | |
| Speedcure | | | | | | | 0.5 | 0.5 | | | | | | | |
| BPO | | | | | | | | | | | | | | | |
| Speedcure | | | | | | | | | 0.5 | 0.5 | | | | | 0.5 |
| BKL | | | | | | | | | | | | | | | |
| Total | 100.5 | 100.5 | 100.5 | 100.5 | 100.5 | 100.5 | 100.5 | 100.5 | 100.5 | 100.5 | 100.0 | 100.0 | 100.0 | 100.5 | 100.5 |

HEx: High Epoxide Matrix
LEx: Low Epoxide Matrix
CMx: Cationic Monomer Matrix

TABLE 13
0.5% TPO-L in hybrid formulation and their curing performance

| | Ctr | Ctr | Ctr | Ctr | Ex | Ex | Ex | Ex | Ctr | Ctr | Ex | Ex | Ex | Ex |
|---|---|---|---|---|---|---|---|---|---|---|---|---|---|---|
| Component | 1A | 2A | 3A | 4A | 1A | 2A | 3A | 4A | 5A | 6A | 5A | 6A | 7A | 8A |
| HEx 1 | 100.0 | | 100.0 | | 100.0 | | 100.0 | | 100.0 | | 100.0 | | 100.0 | |
| LEx 1 | | 100.0 | | 100.0 | | 100.0 | | 100.0 | | 100.0 | | 100.0 | | 100.0 |
| Speedcure 938 | 2.0 | 2.0 | | | | | | | | | | | | |
| Speedcure CPTX | 0.2 | 0.2 | | | | | | | | | | | | |
| Speedcure 992 | | | 5.0 | 5.0 | | | | | | | | | | |
| 2 (comp.) | | | | | 2.0 | 2.0 | | | | | | | | |
| 1 | | | | | | | 2.0 | 2.0 | | | | | | |
| Omnicat 550 | | | | | | | | | 2.0 | 2.0 | | | | |
| 5 | | | | | | | | | | | 2.0 | 2.0 | | |
| 4 (comp.) | | | | | | | | | | | | | 2.0 | 2.0 |
| Propylene | 3.0 | 3.0 | | | 3.0 | 3.0 | 3.0 | 3.0 | 3.0 | 3.0 | 3.0 | 3.0 | 3.0 | 3.0 |
| carbonate | | | | | | | | | | | | | | |
| Total | 105.2 | 105.2 | 105.0 | 105.0 | 105.0 | 105.0 | 105.0 | 105.0 | 105.0 | 105.0 | 105.0 | 105.0 | 105.0 | 105.0 |

Exposed at 405 nm, 10 mW/cm² for 18 s under 10 LPM dry air. Average results from at least 3 tests

| | | | | | | | | | | | | | | |
|---|---|---|---|---|---|---|---|---|---|---|---|---|---|---|
| Acrylate conv. (%) | 66.3 | 61.88 | 40.9 | 56.2 | 67.9 | 68.8 | 56.6 | 57.5 | 33.0 | 15.6 | 55.0 | 54.5 | 63.0 | 66.4 |
| Epoxide conv. (%) | 31.1 | 16.5 | 12.6 | 4.6 | 21.8 | 8.5 | 25.8 | 19.0 | 21.9 | 12.2 | 31.4 | 24.5 | 23.7 | 19.7 |
| 1100 cm⁻¹ peak | 300.7 | 178.3 | 11.8 | 65.0 | 237.8 | 115.6 | 288.9 | 138.5 | 108.0 | 37.0 | 407.5 | 215.5 | 236.8 | 136.7 |
| growth rate (%) | | | | | | | | | | | | | | |

Exposed at 365 nm, 10 mW/cm² for 18 s under 10 LPM dry air. Average results from at least 3 tests

| | | | | | | | | | | | | | | |
|---|---|---|---|---|---|---|---|---|---|---|---|---|---|---|
| Acrylate conv. (%) | 66.3 | 61.7 | 65.2 | 66.4 | 66.1 | 68.3 | 57.5 | 57.9 | 61.3 | 67.0 | 54.4 | 48.1 | 59.0 | 67.5 |
| Epoxide conv. (%) | 31.1 | 16.8 | 29.5 | 17.0 | 33.4 | 18.6 | 32.5 | 22.1 | 36.4 | 23.4 | 32.4 | 22.2 | 29.2 | 23.4 |
| 1100 cm⁻¹ peak | 300.0 | 179.6 | 304.1 | 181.6 | 347.8 | 177.7 | 358.6 | 217.9 | 438.7 | 149.0 | 400.7 | 169.4 | 345.4 | 201.4 |
| growth rate (%) | | | | | | | | | | | | | | |

TABLE 14
0.5% XKm in hybrid formulation and their curing performance

| Component | Ctr 1B | Ctr 2B | Ctr 3B | Ctr 4B | Ctr 5B | Ctr 6B | Ex 5B | Ex 6B | Ex 7B | Ex 8B |
|---|---|---|---|---|---|---|---|---|---|---|
| HEx 2 | 100.0 | | 100.0 | | 100.0 | | 100.0 | | 100.0 | |
| LEx 2 | | 100.0 | | 100.0 | | 100.0 | | 100.0 | | 100.0 |
| Speedcure 938 | 2.0 | 2.0 | | | | | | | | |
| Speedcure CPTX | 0.2 | 0.2 | | | | | | | | |
| Speedcure 992 | | | 5.0 | 5.0 | | | | | | |
| Omnicat 550 | | | | | 2.0 | 2.0 | | | | |
| 5 | | | | | | | 2.0 | 2.0 | | |
| 4 (comp.) | | | | | | | | | 2.0 | 2.0 |
| Propylene carbonate | 3.0 | 3.0 | | | 3.0 | 3.0 | 3.0 | 3.0 | 3.0 | 3.0 |
| Total | 105.2 | 105.2 | 105.0 | 105.0 | 105.0 | 105.0 | 105.0 | 105.0 | 105.0 | 105.0 |

Exposed at 405 nm, 10 mW/cm² for 18 s under 10 LPM dry air. Average results from at least 3 tests

| | | | | | | | | | | |
|---|---|---|---|---|---|---|---|---|---|---|
| Acrylate conv. (%) | 58.9 | 56.7 | 1.7 | −0.5 | 10.9 | 15.8 | 48.9 | 45.1 | 64.6 | 63.4 |
| Epoxide conv. (%) | 31.7 | 18.4 | 0.2 | −2.4 | 15.4 | 17.9 | 31.1 | 25.7 | 29.0 | 16.3 |
| 1100 cm⁻¹ peak | 376.8 | 242.9 | 1.01 | −1.8 | 120.9 | 150.3 | 364.9 | 202.0 | 311.6 | 170.7 |
| growth rate (%) | | | | | | | | | | |

Exposed at 365 nm, 10 mW/cm² for 18 s under 10 LPM dry air. Average results from at least 3 tests

| | | | | | | | | | | |
|---|---|---|---|---|---|---|---|---|---|---|
| Acrylate conv. (%) | 62.5 | 61.7 | 65.2 | 66.4 | 61.3 | 67.0 | 54.4 | 48.1 | 59.0 | 67.5 |
| Epoxide conv. (%) | 35.9 | 16.8 | 29.5 | 16.9 | 36.4 | 23.4 | 32.4 | 22.2 | 29.2 | 23.4 |
| 1100 cm⁻¹ peak | 356.2 | 179.6 | 304.1 | 182.0 | 439.7 | 149.0 | 400.7 | 169.4 | 345.9 | 201.4 |
| growth rate (%) | | | | | | | | | | |

TABLE 15
0.5% TPO in hybrid formulation and their curing performance

| Component | Ctr 1C | Ctr 2C | Ctr 3C | Ctr 4C | Ctr 5C | Ctr 6C | Ex 5C | Ex 6C | Ex 7C | Ex 8C |
|---|---|---|---|---|---|---|---|---|---|---|
| HEx 3 | 100.0 | | 100.0 | | 100.0 | | 100.0 | | 100.0 | |
| LEx 3 | | 100.0 | | 100.0 | | 100.0 | | 100.0 | | 100.0 |
| Speedcure 938 | 2.0 | 2.0 | | | | | | | | |
| Speedcure CPTX | 0.2 | 0.2 | | | | | | | | |
| Speedcure 992 | | | 5.0 | 5.0 | | | | | | |
| Omnicat 550 | | | | | 2.0 | 2.0 | | | | |
| 5 | | | | | | | 2.0 | 2.0 | | |
| 4 (comp.) | | | | | | | | | 2.0 | 2.0 |
| Propylene carbonate | 3.0 | 3.0 | | | 3.0 | 3.0 | 3.0 | 3.0 | 3.0 | 3.0 |
| Total | 105.2 | 105.2 | 105.0 | 105.0 | 105.0 | 105.0 | 105.0 | 105.0 | 105.0 | 105.0 |

Exposed at 405 nm, 10 mW/cm² for 18 s under 10 LPM dry air. Average results from at least 3 tests

| | | | | | | | | | | |
|---|---|---|---|---|---|---|---|---|---|---|
| Acrylate conv. (%) | 60.8 | 60.3 | 59.1 | 65.4 | 61.0 | 63.6 | 55.5 | 48.6 | 68.9 | 67.2 |
| Epoxide conv. (%) | 32.2 | 17.9 | 8.6 | 3.0 | 12.5 | 1.0 | 28.7 | 22.4 | 23.9 | 9.4 |
| 1100 cm⁻¹ peak | 385.2 | 143.5 | 8.0 | 40.5 | 41.0 | 51.0 | 359.6 | 173.9 | 230.3 | 131.8 |
| growth rate (%) | | | | | | | | | | |

Exposed at 365 nm, 10 mW/cm² for 18 s under 10 LPM dry air. Average results from at least 3 tests

| | | | | | | | | | | |
|---|---|---|---|---|---|---|---|---|---|---|
| Acrylate conv. (%) | 62.5 | 61.7 | 65.2 | 166.4 | 61.3 | 67.0 | 54.4 | 48.1 | 54.0 | 67.5 |
| Epoxide conv. (%) | 35.9 | 16.8 | 29.5 | 17.0 | 36.4 | 23.4 | 33.0 | 22.2 | 29.2 | 23.4 |
| 1100 cm⁻¹ peak | 356.2 | 179.6 | 304.1 | 182.0 | 438.7 | 149.0 | 400.7 | 169.4 | 345.9 | 201.4 |
| growth rate (%) | | | | | | | | | | |

TABLE 16
0.5% BPO in hybrid formulation and their curing performance

| Component | Ctr 1D | Ctr 2D | Ctr 3D | Ctr 4D | Ctr 5D | Ctr 6D | Ex 5D | Ex 6D | Ex 7D | Ex 8D |
|---|---|---|---|---|---|---|---|---|---|---|
| HEx 4 | 100.0 | | 100.0 | | 100.0 | | 100.0 | | 100.0 | |
| LEx 4 | | 100.0 | | 100.0 | | 100.0 | | 100.0 | | 100.0 |
| Speedcure 938 | 2.0 | 2.0 | | | | | | | | |
| Speedcure CPTX | 0.2 | 0.2 | | | | | | | | |
| Speedcure 992 | | | 5.0 | 5.0 | | | | | | |
| Omnicat 550 | | | | | 2.0 | 2.0 | | | | |
| 5 | | | | | | | 2.0 | 2.0 | | |
| 4 (comp.) | | | | | | | | | 2.0 | 2.0 |
| Propylene carbonate | 3.0 | 3.0 | | | 3.0 | 3.0 | 3.0 | 3.0 | 3.0 | 3.0 |
| Total | 105.2 | 105.2 | 105.0 | 105.0 | 105.0 | 105.0 | 105.0 | 105.0 | 105.0 | 105.0 |

Exposed at 405 nm, 10 mW/cm² for 18 s under 10 LPM dry air. Average results from at least 3 tests

| | | | | | | | | | | |
|---|---|---|---|---|---|---|---|---|---|---|
| Acrylate conv. (%) | 60.4 | 62.3 | 67.4 | 66.7 | 61.4 | 67.7 | 53.9 | 54.6 | 65.2 | 66.7 |
| Epoxide conv. (%) | 31.7 | 17.8 | 8.0 | −6.2 | 17.2 | 4.2 | 31.3 | 19.9 | 29.4 | 15.3 |
| 1100 cm⁻¹ peak | 398.9 | 167.2 | 9.0 | 61.2 | 167.2 | 189.4 | 372.6 | 212.0 | 289.8 | 147.7 |
| growth rate (%) | | | | | | | | | | |

Exposed at 365 nm, 10 mW/cm² for 18 s under 10 LPM dry air. Average results from at least 3 tests

| | | | | | | | | | | |
|---|---|---|---|---|---|---|---|---|---|---|
| Acrylate conv. (%) | 65.4 | 65.9 | 60.5 | 66.4 | 63.0 | 67.3 | 56.4 | 53.0 | 60.6 | 166.6 |
| Epoxide conv. (%) | 33.1 | 12.5 | 28.1 | 14.7 | 30.6 | 22.6 | 29.9 | 20.6 | 33.2 | 23.4 |
| 1100 cm⁻¹ peak | 368.8 | 160.4 | 314.5 | 157.4 | 398.3 | 229.4 | 365.9 | 214.9 | 323.2 | 153.5 |
| growth rate (%) | | | | | | | | | | |

TABLE 17
0.5% BKL in hybrid formulation and their curing performance

| Component | Ctr 1E | Ctr 2E | Ctr 3E | Ctr 4E | Ctr 5E | Ctr 6E | Ex 5E | Ex 6E | Ex 7E | Ex 8E |
|---|---|---|---|---|---|---|---|---|---|---|
| HEx 5 | 100.0 | | 100.0 | | 100.0 | | 100.0 | | 100.0 | |
| LEx 5 | | 100.0 | | 100.0 | | 100.0 | | 100.0 | | 100.0 |
| Speedcure 938 | 2.0 | 2.0 | | | | | | | | |
| Speedcure CPTX | 0.2 | 0.2 | | | | | | | | |
| Speedcure 992 | | | 5.0 | 5.0 | | | | | | |
| Omnicat 550 | | | | | 2.0 | 2.0 | | | | |
| 5 | | | | | | | 2.0 | 2.0 | | |
| 4 (comp.) | | | | | | | | | 2.0 | 2.0 |
| Propylene carbonate | 3.0 | 3.0 | | | 3.0 | 3.0 | 3.0 | 3.0 | 3.0 | 3.0 |
| Total | 105.2 | 105.2 | 105.0 | 105.0 | 105.0 | 105.0 | 105.0 | 105.0 | 105.0 | 105.0 |

Exposed at 405 nm, 10 mW/cm² for 18 s under 10 LPM dry air. Average results from at least 3 tests

| | | | | | | | | | | |
|---|---|---|---|---|---|---|---|---|---|---|
| Acrylate conv. (%) | 56.4 | 51.5 | 1.29 | 0.3 | −0.5 | 0.5 | 38.4 | 44.1 | 58.4 | 57.3 |
| Epoxide conv. (%) | 32.9 | 24.5 | 0.68 | 1.6 | 14.5 | 3.2 | 35.2 | 27.5 | 31.2 | 22.2 |
| 1100 cm⁻¹ peak | 344.3 | 165.1 | −0.2 | −1.5 | 166.5 | 39.7 | 351.8 | 201.8 | 308.4 | 191.2 |
| growth rate (%) | | | | | | | | | | |

Exposed at 365 nm, 10 mW/cm² for 18 s under 10 LPM dry air. Average results from at least 3 tests

| | | | | | | | | | | |
|---|---|---|---|---|---|---|---|---|---|---|
| Acrylate conv. (%) | 61.9 | 60.4 | 63.6 | 63.8 | 61.7 | 60.1 | 52.5 | 49.6 | 63.2 | 59.9 |
| Epoxide conv. (%) | 31.8 | 19.8 | 34.1 | 21.2 | 37.3 | 27.2 | 35.2 | 24.2 | 35.5 | 23.4 |
| 1100 cm⁻¹ peak | 360.8 | 170.7 | 345.6 | 191.2 | 452.6 | 220.2 | 439.2 | 235.6 | 354.7 | 233.2 |
| growth rate (%) | | | | | | | | | | |

TABLE 18
no radical photoinitiator in hybrid formulation and their curing performance

| Component | Ctr 1F | Ctr 2F | Ctr 3F | Ctr 4F | Ctr 5F | Ctr 6F | Ex 5F | Ex 6F | Ex 7F | Ex 8F |
|---|---|---|---|---|---|---|---|---|---|---|
| HEx 6 | 100.0 | | 100.0 | | 100.0 | | 100.0 | | 100.0 | |
| LEx 6 | | 100.0 | | 100.0 | | 100.0 | | 100.0 | | 100.0 |
| Speedcure 938 | 2.0 | 2.0 | | | | | | | | |
| Speedcure CPTX | 0.2 | 0.2 | | | | | | | | |
| Speedcure 992 | | | 5.0 | 5.0 | | | | | | |
| Omnicat 550 | | | | | 2.0 | 2.0 | | | | |
| 5 | | | | | | | 2.0 | 2.0 | | |
| 4 (comp.) | | | | | | | | | 2.0 | 2.0 |
| Propylene carbonate | 3.0 | 3.0 | | | 3.0 | 3.0 | 3.0 | 3.0 | 3.0 | 3.0 |
| Total | 105.2 | 105.2 | 105.0 | 105.0 | 105.0 | 105.0 | 105.0 | 105.0 | 105.0 | 105.0 |

Exposed at 405 nm, 10 mW/cm² for 18 s under 10 LPM dry air. Average results from at least 3 tests

| | | | | | | | | | | |
|---|---|---|---|---|---|---|---|---|---|---|
| Acrylate conv. (%) | 55.4 | 51.4 | 1.4 | −0.2 | 0.7 | −0.4 | 40.6 | 43.5 | 57.9 | 58.9 |
| Epoxide conv. (%) | 28.4 | 18.2 | 1.7 | 0.7 | 11.5 | 6.5 | 29.3 | 25.4 | 26.0 | 19.1 |
| 1100 cm⁻¹ peak | 383.8 | 105.6 | −1.3 | 4.3 | 140.7 | 35.7 | 404.7 | 222.9 | 288.8 | 158.4 |
| growth rate (%) | | | | | | | | | | |

Exposed at 365 nm, 10 mW/cm² for 18 s under 10 LPM dry air. Average results from at least 3 tests

| | | | | | | | | | | |
|---|---|---|---|---|---|---|---|---|---|---|
| Acrylate conv. (%) | 60.6 | 55.6 | 32.2 | 5.5 | 57.8 | 55.9 | 40.4 | 46.3 | 61.1 | 58.4 |
| Epoxide conv. (%) | 30.3 | 20.8 | 27.4 | 31.9 | 36.8 | 30.9 | 29.9 | 30.7 | 32.5 | 25.0 |
| 1100 cm⁻¹ peak | 368.9 | 170.1 | 352.4 | 144.2 | 403.3 | 222.5 | 330.1 | 232.5 | 357.5 | 203.5 |
| growth rate (%) | | | | | | | | | | |

TABLE 19
No radical initiator in cationic formulations and their properties

| Component | Ctr 7A | Ctr 8A | Ex 9A | Ex 10A | Ex 11A | Ctr 9A | Ex 12A |
|---|---|---|---|---|---|---|---|
| CMx | 100.0 | 100.0 | 100.0 | 100.0 | 100.0 | 100.0 | 100.0 |
| Speedcure 938 | 1.0 | | | | | | |
| Speedcure CPTX | 0.2 | | | | | | |
| Speedcure 992 | | 2.5 | | | | | |
| 2 (comp.) | | | 1.0 | | | | |
| 4 (comp.) | | | | 1.0 | | | |
| 1 | | | | | 1.0 | | |
| Omnicat 550 | | | | | | 1.0 | |
| 5 | | | | | | | 1.0 |
| Propylene carbonate | 1.5 | | 1.5 | 1.5 | 1.5 | 1.5 | 1.5 |
| Total | 102.7 | 102.5 | 102.5 | 102.5 | 102.5 | 102.5 | 102.5 |

Exposed at 405 nm, 10 mW/cm² for 18 s under 10 LPM dry air. Average results from at least 3 tests

| | | | | | | | |
|---|---|---|---|---|---|---|---|
| Epoxide conv. (%) | 26.1 | 0.71 | 20.0 | 24.5 | 26.7 | 6.9 | 29.1 |
| 1100 cm⁻¹ peak | 7644.9 | 0.35 | 591.2 | 626.2 | 601.7 | 146.6 | 715.2 |
| growth rate (%) | | | | | | | |

Exposed at 365 nm, 10 mW/cm² for 18 s under 10 LPM dry air. Average results from at least 3 tests

| | | | | | | | |
|---|---|---|---|---|---|---|---|
| Epoxide conv. (%) | 29.8 | 20.4 | 27.7 | 27.5 | 30.5 | 29.4 | 30.9 |
| 1100 cm⁻¹ peak | 495.5 | 527.7 | 737.9 | 736.5 | 633.2 | 756.0 | 727.5 |
| growth rate (%) | | | | | | | |

TABLE 20
0.5% TPO-L in cationic formulations and their properties

| Component | Ctr 7B | Ctr 8B | Ex 9B | Ex 10B | Ex 11B | Ctr 9B | Ex 12B |
|---|---|---|---|---|---|---|---|
| CMx 1 | 100.0 | 100.0 | 100.0 | 100.0 | 100.0 | 100.0 | 100.0 |
| Speedcure 938 | 1.0 | | | | | | |
| Speedcure CPTX | 0.2 | | | | | | |
| Speedcure 992 | | 2.5 | | | | | |
| 2 (comp.) | | | 1.0 | | | | |
| 4 (comp.) | | | | 1.0 | | | |
| 1 | | | | | 1.0 | | |
| Omnicat 550 | | | | | | 1.0 | |
| 5 | | | | | | | 1.0 |
| Propylene carbonate | 1.5 | | 1.5 | 1.5 | 1.5 | 1.5 | 1.5 |
| Total | 102.7 | 102.5 | 102.5 | 102.5 | 102.5 | 102.5 | 102.5 |

Exposed at 405 nm, 10 mW/cm² for 18 s under 10 LPM dry air. Average results from at least 3 tests

| | | | | | | | |
|---|---|---|---|---|---|---|---|
| Epoxide conv. (%) | 21.7 | −1.4 | 6.6 | 14.7 | 22.9 | 2.5 | 23.9 |
| 1100 cm⁻¹ peak | 389.9 | 3.1 | 261.9 | 371.5 | 558.7 | 102.5 | 598.0 |
| growth rate (%) | | | | | | | |

Exposed at 365 nm, 10 mW/cm² for 18 s under 10 LPM dry air. Average results from at least 3 tests

| | | | | | | | |
|---|---|---|---|---|---|---|---|
| Epoxide conv. (%) | 28.8 | 13.88 | 20.6 | 25.0 | 31.6 | 25.6 | 33.3 |
| 1100 cm⁻¹ peak | 431.8 | 395.5 | 565.0 | 558.5 | 654.7 | 615.3 | 655.5 |
| growth rate (%) | | | | | | | |

TABLE 21
0.5% BKL in cationic formulations and their properties

| Component | Ctr 7C | Ctr 8C | Ex 9C | Ex 10C | Ex 11C | Ctr 9C | Ex 12C |
|---|---|---|---|---|---|---|---|
| CMx 2 | 100.0 | 100.0 | 100.0 | 100.0 | 100.0 | 100.0 | 100.0 |
| Speedcure 938 | 1.0 | | | | | | |
| Speedcure CPTX | 0.2 | | | | | | |
| Speedcure 992 | | 2.5 | | | | | |
| 2 (comp.) | | | 1.0 | | | | |
| 4 (comp.) | | | | 1.0 | | | |
| 1 | | | | | 1.0 | | |
| Omnicat 550 | | | | | | 1.0 | |
| 5 | | | | | | | 1.0 |
| Propylene carbonate | 1.5 | | 1.5 | 1.5 | 1.5 | 1.5 | 1.5 |
| Total | 102.7 | 102.5 | 102.5 | 102.5 | 102.5 | 102.5 | 102.5 |

Exposed at 405 nm, 10 mW/cm² for 18 s under 10 LPM dry air. Average results from at least 3 tests

| | | | | | | | |
|---|---|---|---|---|---|---|---|
| Epoxide conv. (%) | 27.0 | −3.0 | 21.2 | 21.6 | 26.6 | 5.4 | 27.0 |
| 1100 cm⁻¹ peak | 768.9 | 7.0 | 473.0 | 578.6 | 599.3 | 151.6 | 649.4 |
| growth rate (%) | | | | | | | |

Exposed at 365 nm, 10 mW/cm² for 18 s under 10 LPM dry air. Average results from at least 3 tests

| | | | | | | | |
|---|---|---|---|---|---|---|---|
| Epoxide conv. (%) | 30.4 | 24.1 | 30.7 | 29.5 | 32.2 | 31.4 | 33.5 |
| 1100 cm⁻¹ peak | 491.6 | 605.9 | 649.4 | 665.8 | 657.4 | 704.4 | 660.6 |
| growth rate (%) | | | | | | | |

### Matrix and Formulation Preparation in Tables 12 to 21

Matrixes: In a 1000 mL of metal can, formulation matrix was charged according to the percentage in Table 12. The 1000-1005 g mixture of each matrix sample was prepared and mixed by mechanical mixer for about 1 hour around 60° C. until the solution became clear. Formulations: In a white max 50 jar from FlackTek Inc., photoinitiator and propylene carbonate were loaded at first, mixed by hand with stainless steel spatula, placed in 60° C. oven for about 1 h, mixed again until it became clear. Then, formulation matrix was charged according to the percentage in one of Table 13 to 21. The 51.25.5-52.60 g mixture of each sample was prepared and mixed for 3 minutes at 3000 RPM in Speed Mixer from FleackTec Inc. Then, all jars were placed in 60° C. oven for about 2 h, taken out and immediately mixed for another 2 minutes until the solution became clear.

### FTIR Test

A Fourier Transform Infrared (FTIR) with an Attenuated Total Reflection (ATR) setup was used. All polymerization rate measurements were performed using Nicolet iS50 FT-IR Spectrometer from Thermo Scientific, equipped with a standard DLaTGS detector. A lamp holder for the ART platform of FTIR unit can be customized and printed from Arkema N3xtDimention® engineered resin N3D-TOUGH784 in order to ensure precisely fit of a 365 nm lamp Accucure ULM-2-365 or a 405 nm lamp Accucure ULM-2-405 from Digital Light Labs. On the bottom of this lamp holder, a dry air channel is built in to allow air uniformly blowing over sample surface, the gas flow rate can be controlled over a rotameter. The LED light was manually triggered by Ultraviolet illumination & Measurement System. LED light exposure can be programed by AccuCure software. For measurement, 25 μL of liquid sample was placed in the center of an ATR crystal. 3 mil of thin film was prepared by a customized coating applicator (3 mil WFM, G1046 from BYK). The LED lamp with holder was place on the top of ART platform. Then FTIR scan was initiated to collect liquid IR spectrum at first. Each IR spectrum of a specific exposure time at 10 mW/cm² of LED light were collected. Measurements of acrylate conversion were taken at the peak height under the reference peak around 1727 cm⁻¹; the acrylate peak of SR833S at approximately 1407 cm⁻¹, the epoxide peak of UviCure S105 at approximately 790 cm⁻¹, and the oxetane peak of UViCure S130 at approximately 970 cm⁻¹ were also measured. Since the ring opening of both epoxide and oxetane generated C—O—C bond, the growth of C—O—C IR peak height at ~1100 cm⁻¹ was monitored as well. The resulting of peak 1100 cm⁻¹ growth rate could be calculated to assess total cationic cure speed. Peak heights were determined using the same baseline where a baseline is chosen to be the two lowest points between 600 cm⁻¹ and 1800 cm⁻¹. The peak height under the peak and above the baseline was then determined. The integration limits for liquid and the cured sample are not identical but are similar, especially for the reference peak.

The ratios of the acrylate peak height, epoxide peak height, ring opening peak height from both epoxide and oxetane to the reference peak height were determined for both the liquid and the cured samples. Degree of cure or conversion or peak growth rate, expressed as percentage reacted acrylate or epoxide or ring opening of both epoxide and oxetane, was calculated from the equation below:

$$Conversion ( \% ) = [ ( {R}_{liq} - {R}_{c} ) \times 100 ] / {R}_{liq} \\ Peak growth rate ( \% ) = [ ( {R}_{c} - {R}_{liq} ) \times 100 ] / {R}_{liq}$$

Where R_{liq} is the peak height ratio of the liquid sample and R_{c} is the peak height ratio of the LED cured sample. The resulting acrylate and epoxide conversions or C—O—C growth rates were collected and listed in Tables 13 to 21, plotted in FIG. 1 to 8 and FIG. 10.

### UV Vis Spectra Measurement

UV-Vis spectrum of each sample was taken with a Shimadzu UV1800 spectrophotometer using a 1.0 cm path of cuvette quartz cell in accordance with ASTM E169-04, and scanning the spectrum over the wavelength range of 450 to 200 nm. The measuring cell was filled a 10 ppm photoinitiator in acetonitrile solution to ensure that the observed absorbance did not exceed 1.0 in the range of the spectrum for which absorbance values were desired.

### Working-Curve Measurement

Working-curves were printed on the 405 nm~3 mW/cm² Flashforge Hunter DLP printer or on the 405 nm~12 mW/cm² B9 Core 550 DLP printer from B9Creation. Various energy dosages were irradiated in sections of the build area (without a build platform installed), resulting in individual squares or thin films being cured. The thickness of the individual thin films were measured with a low force digital caliper+comparator stand from Mitutoyo to determine the cure depths. A plot of the cure depths vs. the logarithm of energy dosages was used to determine the critical exposure (E_{c}, mJ/cm²) and penetration depth (Dₚ, mils).

### Preparation of Tensile Test Parts

Diagnostic parts were printed on the 405 nm B9 Core 550 DLP 3D printer with an irradiance of approximately 12 mW/cm². ASTM D638-14 Type IV tensile dog bones were designed in CAD software, and exported to STL files to allow for 3D printing of the diagnostic parts. Parts were printed in the XY plane, directly on the build platform without support structures at 50 microns layer thickness. the energy dosage that was used per 50 micron layer for printing was 50 mJ/cm² for Ex 17 and 25 mJ/cm² for Ex 18. These energy dosages were determined from working-curve data allowing for 150 microns of cure depth. Slight adjustments were made based on iterative experiments to maximize printability & resolution.

Parts were post-cured for 20 minutes per side in a Sprintray ProCure UV post-curing apparatus. Irradiance measurements of the post-curing unit at various wavelengths are shown below, which were collected with an Ophir Starbright power meter coupled with a PD300RM-UV radiometer.

TABLE 22
Irradiance measurements of the post-curing unit at various wavelengths
Following UV post-cure, samples were conditioned for seven days
before test following ASTM D618 - 13 - Procedure A.

| Wavelength (nm) | 250 | 315 | 340 | 390 | 400 | 405 |
|---|---|---|---|---|---|---|
| Irradiance | 45.4 | 23.4 | 19.1 | 13.4 | 12.0 | 11.4 |
| (mW/cm²) | | | | | | |

### Mechanical Testing of 3D Printed Articles:

Samples were tested following ASTM D638-14 with an Instron 5966 universal testing apparatus equipped with 5 kN wedge grips. A pull rate of 5 mm/min was used, and a static axial clip-on extensometer was utilized for determining Young's Modulus.

### 4.3 Results and Discussion

### 4.3.1. Cure Performance in Hybrid Systems

As listed in Table 13, acrylate and epoxide at 405 nm illustrated in following FIGS. 1 and 2.

Results showed in either high epoxide (HE) or low epoxide (LE) of hybrid systems: 1) all new cationic photoinitiators showed the better acrylate cure than Omnicat 550; 2) all new cationic photoinitiators showed both of the better epoxide and total cationic cure than Speedcure 992. Epoxide cure of photoinitiators 5, 1 and 4 (comp.) was better than that from Omnicat 550 as well, matched with the control sample SC938/CPTX.

As listed in Table 13 to 18, the effect of different radical photoinitiator or no radical photoinitiator on acrylate, epoxide and total cationic cure at 405 nm illustrated in following FIGS. 3, 4 and 5.

Results showed in either high epoxide (HE) or low epoxide (LE) of hybrid systems: 1) Both 5 and 4 (comp.) showed high acrylate cure with or without radical photoinitiator. In presence of shorter wavelength of radical initiator BKL or low 405 nm absorption of XKm, even without any radical initiator, both 4 (comp.) and CPTX-CPT could cure acrylate very well like SC938/CPTX. 2) Both 5 and 4 (comp.) showed better epoxide cure and total cationic cure than Omnicat 550 and SC992 either in presence or absence of radical photoinitiator. Overall, both 5 and 4 (comp.) in hybrid systems performed well and matched with SC938/CPTX.

At a short wavelength of LED exposure, such as 365 nm, those new cationic photoinitiators performed similarly to SC938/CPTX, Omnicat 550 and SC992 as listed in Tables 13 to 18.

### 4.3.2. Cure Performance in Cationic Systems

As listed in Table 19 to 21, epoxide, oxetane and total cationic cure at 405 nm illustrated in following FIGS. 6, 7 and 8.

Results showed in cation systems: 1) All four new cationic photoinitiators 2 (comp.), 4 (comp.), 1 and 5 showed better epoxide cure, oxetane cure as well as total cationic cure than both Omnicat550 and SC992. Among them, 5 and 1 performed slightly better than 2 (comp.) or 4 (comp.). Neither radical photoinitiator BKL nor TPO-L could promote cationic photopolymerization. As the matter of fact, radical photoinitiator slowed down cationic cure and TPO-L slowed down the most.

At a short wavelength of LED exposure, such as 365 nm, those new cationic photoinitiators performed very similarly to Omnicat 550 and SC938/CPTX, slightly better than SC992 as listed in Tables 19 to 21.

### 4.3.3 3D Printability in Hybrid System

UV spectra: As showed in FIG. 9, UV spectra of four new sulfonium salts were compared with Omnicat 550, Speedcure 992S (>99% active ingredient) and Speedcure 938. At 405 nm, the UV absorption decreased by the order 5>1>2 (comp.) ~4 (comp.), they all were much higher than Omnicat 550 and SC 992S. SC938 did not have any absorption at all over 310 nm wavelength.

Formulations for 3D printing at 405 nm: A typical hybrid system was selected to evaluate the printability of both 5 and 4 (comp.) in comparison with Omnicat 550 and SC992 as showed in Table 23. Working curve data were measured from either 3.1 mW and 405 nm Flashforge Hunter DLP printer or −10 mW and 405 nm B9 Core 550 DLP printer

TABLE 23
Formulations for 405 nm DLP printers and their properties from printed parts

| Components | Ctr 10 | Ex 13 | Ex14 | Ctr 11 | Ex 15 | Ex 16 | Ex 17 | Ex 18 |
|---|---|---|---|---|---|---|---|---|
| Eponex 1510 | 25.00 | 25.00 | 25.00 | 25.00 | 10.00 | 10.00 | 10.00 | 10.00 |
| UviCure S105 | 35.00 | 35.00 | 35.00 | 35.00 | 30.00 | 30.00 | 30.00 | 30.00 |
| UviCure S130 | 10.00 | 10.00 | 10.00 | 10.00 | 10.00 | 10.00 | 10.00 | 10.00 |
| SR499 | 20.00 | 20.00 | 20.00 | 20.00 | 35.00 | 35.00 | 35.00 | 35.00 |
| SR259 | 10.00 | 10.00 | 10.00 | 10.00 | 15.00 | 15.00 | 15.00 | 15.00 |
| SC BKL | 1.00 | 1.00 | 1.00 | 1.00 | 2.00 | 2.00 | | |
| TPO-L | | | | | | | 0.50 | 0.35 |
| SC992 | 4.00 | | | | | | | |
| 5 | | 1.60 | | | 0.64 | | 0.48 | |
| 4 (comp.) | | | 1.60 | | | 1.10 | | 1.10 |
| Omnicat 550 | | | | 1.60 | | | | |
| Propylene | | 2.40 | 2.40 | 2.40 | 0.96 | 1.65 | 0.72 | 1.65 |
| Carbonate | | | | | | | | |
| BYK-333 | | | | | 1.00 | 1.00 | 1.00 | 1.00 |
| Total | 105.00 | 105.00 | 105.00 | 105.00 | 104.60 | 105.75 | 102.70 | 104.10 |
| Dp (mils) | not | 1.2 | 2.8 | not | 3 | 5.5 | 4.6 | 8.2 |
| Ec (mJ/cm²) | printable | 21.3 | 45.4 | printable | 27.4 | 26.1 | 16.2 | 8.3 |
| Exposure time/layer (s)^{a} | 20 | 15 | 15 | 20 | | | | |
| Printing energy (mJ/cm²) ^{b} | | | | | 10 | 10 | 40 | 40 |
| Tensile Strength (MPa) | | | | | | | 11.4 | 9.8 |
| Elongation at Break (%) | | | | | | | 9.4 | 17.2 |
| Tensile Modulus (MPa) | | | | | | | 566 | 601 |
| Energy at Break (J) | | | | | | | 0.44 | 0.73 |

^{a}Data collect from 3.1 mW and 405 nm Flashforge Hunter DLP printer.
^{b} Data collect from ~10 mW and 405 nm B9 Core 550 DLP printer.

Working curve square films of each formulation were printed from either 3.1 mW and 405 nm Flashforge Hunter DLP printer or −10 mW and 405 nm 1B9 Core 550 DLP printer, and listed in Table 23 along with printing exposure condition. As expected, both SC992 (Ctr 10) and Omnicat 550 (Ctr 11) were not printable even with over exposure time. Two new sulfonium salts were printed well, and usually 5 at lower concentration could provide lower Op and higher Ec than 4 (comp.) did due to its high absorption at 405 nm in FIG. 9.

In a 10LPM flow rate of dry air, both acrylate and epoxide cure of formulation Ex 17 and Ex 18 could cure well at 10 mW of 405 nm LED as showed in FIG. 10. A set of tensile parts were successfully printed from formulation Ex 17 and Ex 18 by using ~10 mW and 405 nm 9 Core 550 DLP printer, the post UV cured parts provided a set of desirable tensile properties data as listed in Table 23.

### Example 5: Cure and Colour Measurements of Coatings in the Presence of Anti-Yellowing Additives

The following hybrid formulation (control) was prepared for assessment of cure and colour:

TABLE 24
Composition of the hybrid formulation (control).

| Component | wt parts |
|---|---|
| Eponex 1510 | 10 |
| UviCure S105 | 30 |
| UviCure S130 | 10 |
| Sartomer SR454 | 35 |
| Sartomer SR259 | 15 |
| Photoinitiator (5) | 1 |

Anti-yellowing additives were then further added to the control at levels indicated in Table 25. The test formulations were coated on Leneta 3NT-31 paper substrate (coating thickness 50 μm) and passed under a LED 395 nm light source (5 W/cm2, 10 passes). Colour measurements of the cured coatings were taken immediately after cure using PCE colorimeter (Model #PCE-CSM3).

TABLE 25
Effect of anti-yellowing additives on cure and coating colour

| | additive | Curing | | | |
|---|---|---|---|---|---|
| Additive | wt % | assessment | L* | a* | b* |
| None | — | | 92.48 | −0.31 | 1.14 |
| (White Paper Substrate) | | | | | |
| None | 0.0% | Fully cured | 77.99 | 14.04 | 17.04 |
| (Control formulation) | | | | | |
| Speedcure EDB | 0.1% | Fully cured | 78.65 | 10.84 | 16.55 |
| BHT | 0.1% | Fully cured | 78.13 | 12.39 | 15.91 |
| (CAS# 128-37-0) | 0.5% | Fully cured | 80.98 | 8.86 | 13.03 |
| | 1.0% | Fully cured | 82.48 | 6.77 | 12.00 |
| TEMPO | 0.1% | Partly cured | 80.39 | 14.05 | 17.28 |
| (CAS# 2564-83-2) | | | | | |
| Poly(acrylonitrile- | 1.0% | Fully cured | 78.44 | 13.38 | 16.99 |
| co-butadiene), | | | | | |
| dicarboxy terminated | | | | | |
| (CAS# 68891-46-3) | | | | | |
| (Phenylthio)acetic acid | 0.1% | Fully cured | 83.76 | 7.49 | 13.93 |
| | 0.5% | Fully cured | 88.46 | 1.78 | 12.28 |
| TMEI | 0.1% | Fully cured | 83.32 | 6.12 | 13.10 |
| (CAS# 36196-44-8) | 0.5% | Fully cured | 86.47 | 3.67 | 14.40 |
| | 1.0% | Fully cured | 88.58 | −0.74 | 18.52 |

### Example 6: Solubility of Phosphoinitiators in Propylene Carbonate

Solubility of selected sulfonium salts in propylene carbonate at ambient temperature (20-25° C.) was determined.

TABLE 26
Solubility of compounds in propylene carbonate at 20-25° C.

| sample | Solubility in propylene carbonate | |
|---|---|---|
| Omnicat 550 | 25 | wt % |
| (comp.) | | |
| 1 | >50 | wt % |
| 2 (comp.) | >48 | wt % |
| 4 (comp.) | >47 | wt % |
| 5 | 42 | wt % |
| 50 | 5.5 | wt % |

1. A method of curing a composition comprising a cationically-polymerizable compound and a photoinitiator of formula (I): 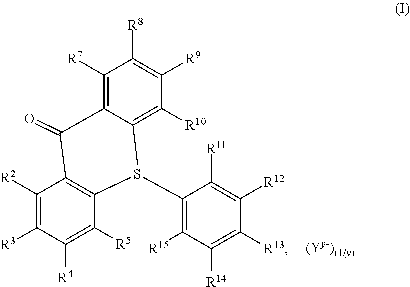
wherein:
Y is an anion, the valency of which is y,
either R¹² and R¹³ are linked with each other so that the 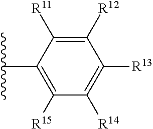
group represents 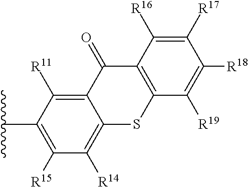
wherein
R¹⁶, R¹⁷, R¹⁸ and R¹⁹ are independently chosen among H, a halogen, a (C₁-C₆) linear or branched alkyl group, a (C₁-C₆) linear or branched alkoxy group, a —O—(CH₂)ᵢ—COOR²⁸ or —(CH₂)ᵢ—CH—(COOR²⁸)₂ group wherein i is 1 or 2 and R²⁸ is H or a (C₁-C₄) linear or branched alkyl group, and
R¹¹, R¹⁴ and R¹⁵ are independently H, a halogen, a (C₁-C₆) linear or branched alkyl group, a (C₁-C₆) linear or branched alkoxy group and —S-Ph-C(═O)-Ph
or R¹² and R¹³ are not linked with each other, and
R¹¹, R¹² and R¹³ are independently chosen from H, a halogen, a (C₁-C₆) linear or branched alkyl group, a (C₁-C₆) linear or branched alkoxy group, a pyrrolidin-1-yl, a -L-Ph¹ group wherein L is a single bond, CH₂ or O and Ph¹ is a phenyl group optionally substituted by one or several substituents chosen from a halogen, a (C₁-C₆) linear or branched alkyl group and a (C₁-C₆) linear or branched alkoxy group, provided that at least one R¹¹, R¹² and R¹³ is a -L-Ph¹ group, and
R¹⁴ and R¹⁵ are independently H, a halogen, a (C₁-C₆) linear or branched alkyl group, a (C₁-C₆) linear or branched alkoxy group and —S-Ph-C(═O)-Ph,
R³ is chosen from, H, a halogen, a (C₁-C₆) linear or branched alkyl group, a (C₁-C₆) linear or branched alkoxy group, a —O—(CH₂)ₗ—COOR³¹ group and a —(CH₂)ₗ—CH—(COOR³¹)₂ group, wherein l is 1 or 2 and R³¹ is H or a (C₁-C₄) linear or branched alkyl group,
R², R⁴, R⁵, R⁷, R⁸, R⁹ and R¹⁰ are independently chosen from H, a halogen, a (C₁-C₆) linear or branched alkyl group, a (C₁-C₆) linear or branched alkoxy group and a —O—(CH₂)ₘ—COOR³² or —(CH₂)ₘ—CH—(COOR³²)₂ group wherein m is 1 or 2 and R³² is H or a (C₁-C₄) linear or branched alkyl group,
the method comprising irradiating the composition with at least one light source having a maximum output wavelength in the range of 350 to 460 nm.

2. The method according to claim 1, wherein, in formula (I) of the photoinitiator: R¹² and R¹³ are linked with each other so that the group 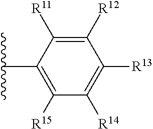 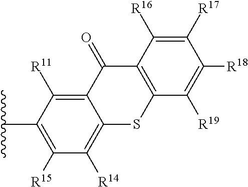
so that the photoinitiator has formula (IV): 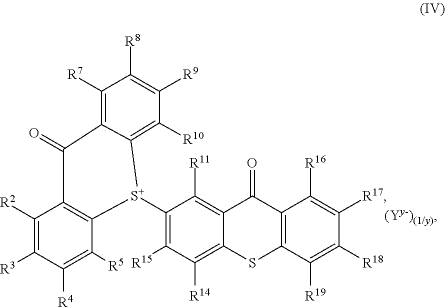
wherein R², R³, R⁴, R⁵, R⁷, R⁸, R⁹, R¹⁰, R¹¹, R¹⁴, R¹⁵, R¹⁶, R¹⁷, R¹⁸, R¹⁹, Y and y are as defined in claim 1.

3. The method according to claim 2, wherein:
R², R⁴, R⁷, R⁸, R⁹ and R¹⁰ represent H, R⁵ is chosen from H, a halogen, a (C₁-C₆) linear or branched alkyl group, and R³ is chosen from a halogen, a (C₁-C₆) linear or branched alkyl group, a (C₁-C₆) linear or branched alkoxy group, a —O—(CH₂)ₗ—COOR³¹ group and a —(CH₂)ₗ—CH—(COOR³¹)₂ group, wherein l is 1 or 2 and R³¹ is H or a (C₁-C₄) linear or branched alkyl group,
or
R³, R⁴, R⁷, R⁸, R⁹ and R¹⁰ are H and R² and R⁵ are independently chosen from a halogen, a (C₁-C₆) linear or branched alkyl group, a (C₁-C₆) linear or branched alkoxy group and a —O—(CH₂)ₘ-COOR³² or —(CH₂)ₘ—CH—(COOR³²)₂ group wherein m is 1 or 2 and R³² is H or a (C₁-C₄) linear or branched alkyl group.

4. The method according to claim 2, wherein, in the formula of the photoinitiator:
R¹⁶, R¹⁸, R¹¹, R¹⁴ and R¹⁵ represent H, R¹⁹ is chosen from H, a halogen, a (C₁-C₆) linear or branched alkyl group and R¹⁷ is chosen from a halogen, a (C₁-C₆) linear or branched alkyl group, a (C₁-C₆) linear or branched alkoxy group, a —O—(CH₂)ᵢ—COOR²⁸ or —(CH₂)ᵢ—CH—(COOR²⁸)₂ group wherein i is 1 or 2 and R²⁸ is H or a (C₁-C₄) linear or branched alkyl group,
or
R¹⁷, R¹⁸, R¹¹, R¹⁴ and R¹⁵ are H and R¹⁶ and R¹⁹ are independently chosen from a halogen, a (C₁-C₆) linear or branched alkyl group, a (C₁-C₆) linear or branched alkoxy group and a —O—(CH₂)ᵢ—COOR²⁸ or —(CH₂)ᵢ—CH—(COOR²⁸)₂ group wherein i is 1 or 2 and R²⁸ is H or a (C₁-C₄) linear or branched alkyl group.

5. The method according to claim 2, wherein, in formula (IV):
R⁷ and R¹¹ are identical,
R⁹ and R¹⁵ are identical,
R¹⁰ and R¹⁴ are identical,
R² and R¹⁶ are identical,
R³ and R¹⁷ are identical,
R⁴ and R¹¹ are identical, and
R⁵ and R¹⁹ are identical,
and the photoinitiator has formula (V): 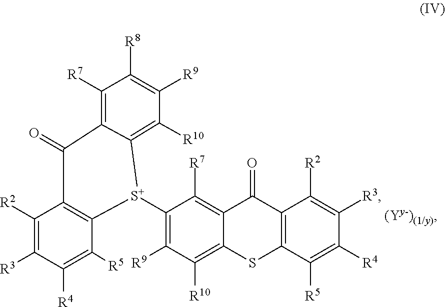
wherein:
R⁷, R⁹ and R¹⁰ are independently H, a halogen, a (C₁-C₆) linear or branched alkyl group or a (C₁-C₆) linear or branched alkoxy group,
R², R³, R⁴ and R⁵ are independently chosen among H, a halogen, a (C₁-C₆) linear or branched alkyl group, a (C₁-C₆) linear or branched alkoxy group, a —O—(CH₂)ₘ—COOR³² or —(CH₂)ₘ—CH—(COOR³²)₂ group wherein m is 1 or 2 and R³² is H or a (C₁-C₄) linear or branched alkyl group, and
R¹, Y and y are as defined in claim 1.

6. The method according to claim 2, wherein the photoinitiator has formula (1), (5), (6), (40), (44), (45), (46), (47), (48) or (49): 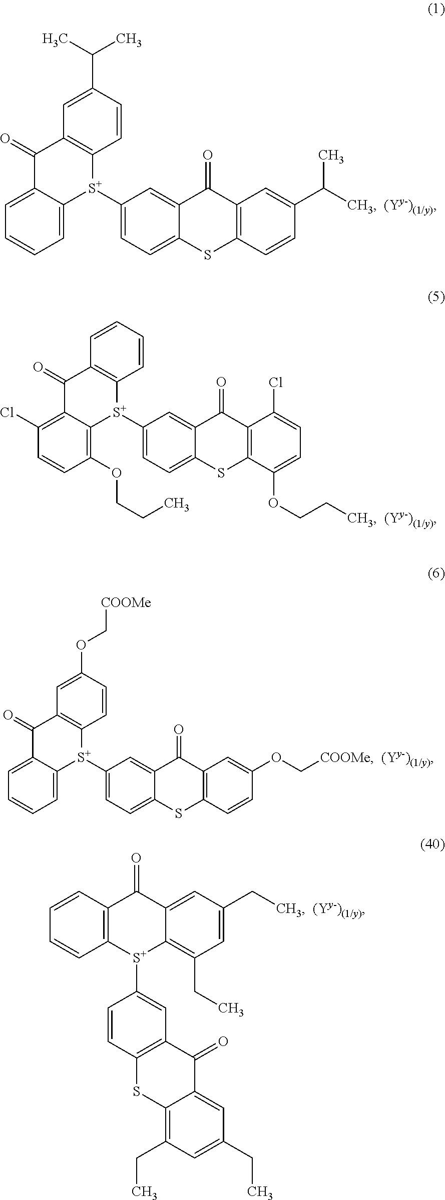 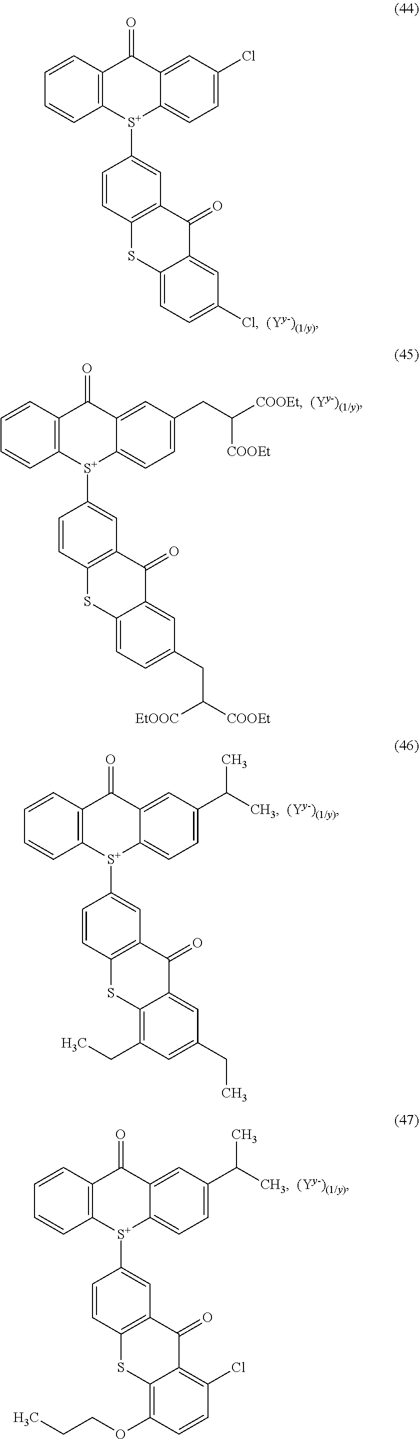 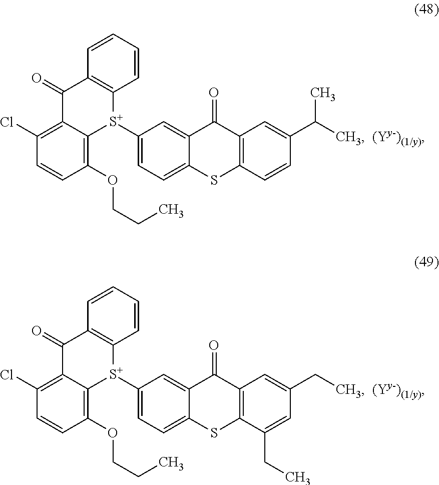
wherein Y and y are as defined in claim 1.

7. The method according to claim 1, wherein, in formula (I) of the photoinitiator, R¹² and R¹³ are not linked with each other, so that the photoinitiator has formula (VII): 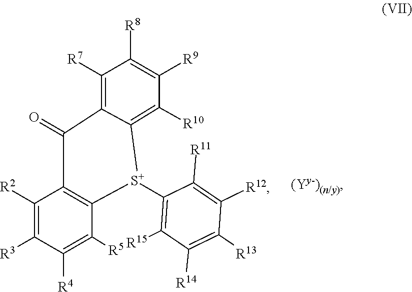
wherein R², R³, R⁴, R⁵, R⁷, R⁸, R⁹, R¹⁰, R¹¹, R¹², R¹³, R¹⁴, R¹⁵, Y and y are as defined in claim 1.

8. The method according to claim 7, wherein the photoinitiator has formula (VIII): 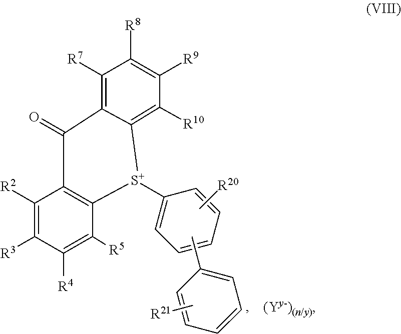
wherein:
R², R³, R⁴, R⁵, R⁷, R⁸, R⁹, R¹⁰, Y and y are as defined in claim 1, and
R²⁰ and R²¹ are independently chosen from H, a halogen, a (C₁-C₆) linear or branched alkyl group, a (C₁-C₆) linear or branched alkoxy group.

9. The method according to claim 8, wherein R²¹ is a (C₁-C₆) linear or branched alkoxy group.

10. The method according to claim 7, wherein the photoinitiator has formula (12), (16), (26), (28), (32) or (33): 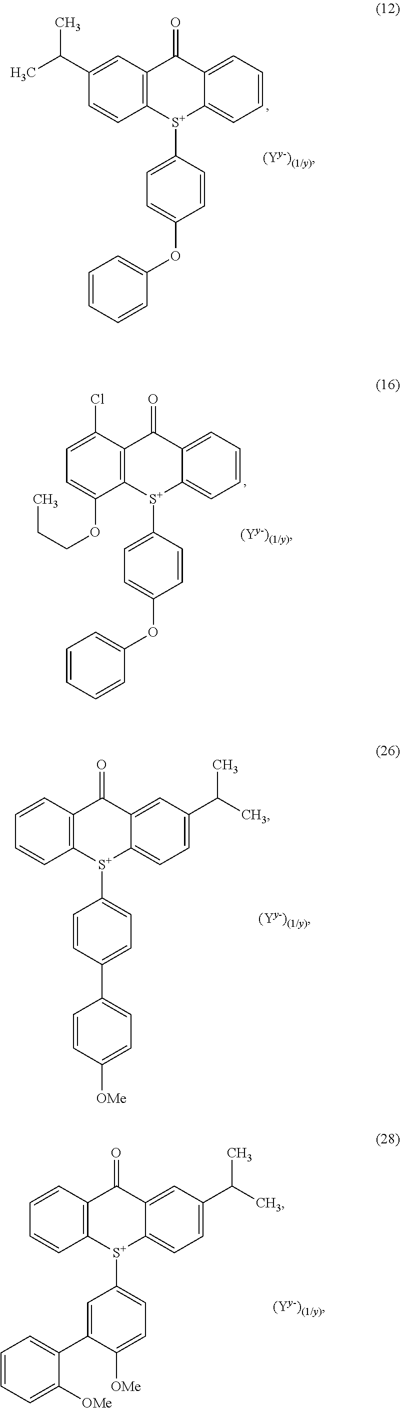 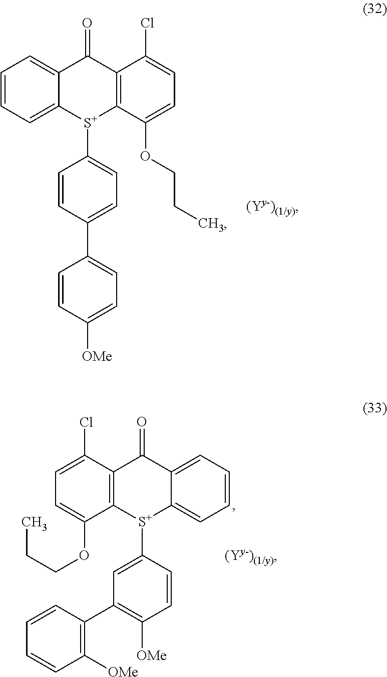
wherein Y and y are as defined in claim 1.

11. The method according to claim 1, wherein the light source has a maximum output wavelength from 380 to 430 nm.

12. The method according to claim 1, wherein the light source is a light-emitting diode (LED), or a broadband lamp with an optical filter that that limits emission to wavelengths in the range of 350 to 460 nm.

13. The method according to claim 1, wherein the cationically-polymerizable compound comprises at least one compound selected from epoxide, oxetane, oxolane, cyclic acetal, cyclic lactone, thiiranes, thiethanes, spiro orthoester, vinyl ether, and mixtures thereof.

14. The method according to claim 1, wherein the composition further comprises a radically-polymerizable compound and optionally a radical photoinitiator.

15. The method according to claim 1, wherein the composition further comprises an anti-yellowing agent.

16. A cured product obtained according to the method according to claim 1.

17. A method for the preparation of a 3D-printed article comprising the method according to claim 1.

18. The method according to claim 17, wherein the method comprises the following steps:
a) depositing the first layer of a composition onto a surface;
b) curing the first layer at least partially, to provide a cured layer;
c) depositing a second layer of the composition onto the cured first layer;
d) curing the second layer, at least partially, to provide a cured second layer adhered to cured first layer;
e) repeating steps c) and d) a desired number of times to build up the 3D-printed article, and optionally a post-curing step of heating or microwave irradiating the 3D-printed article.

19. The method according to claim 18, wherein the method comprises the following steps:
a) providing a carrier and an optically transparent member having a build surface, the carrier and build surface defining a build region therebetween;
b) filling the build region with a composition;
c) continuously or intermittently curing part of the composition in the build region to form a cured composition;
d) continuously or intermittently advancing the carrier away from the build surface to form the 3D-printed article from the cured composition, and
and
optionally a post-curing step of heating or microwave irradiating the 3D-printed article.

20. A 3D printed article obtained with the method according to claim 17.

21. A compound of formula (VIII): 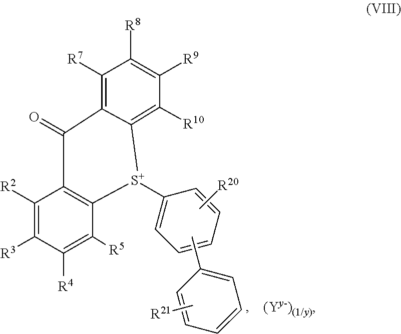
wherein:
Y is an anion, the valency of which is y,
R³ is chosen from, H, a halogen, a (C₁-C₆) linear or branched alkyl group, a (C₁-C₆) linear or branched alkoxy group, a —O—(CH₂)ₗ—COOR³¹ group and a —(CH₂)ₗ—CH—(COOR³¹)₂ group, wherein l is 1 or 2 and R³¹ is H or a (C₁-C₄) linear or branched alkyl group,
R², R⁴, R⁵, R⁷, R⁸, R⁹ and R¹⁰ are independently chosen from H, a halogen, a (C₁-C₆) linear or branched alkyl group, a (C₁-C₆) linear or branched alkoxy group and a —O—(CH₂)ₘ—COOR³² or —(CH₂)ₘ—CH—(COOR³²)₂ group wherein m is 1 or 2 and R³² is H or a (C₁-C₄) linear or branched alkyl group, and
R²⁰ and R²¹ are independently chosen from H, a halogen, a (C₁-C₆) linear or branched alkyl group, a (C₁-C₆) linear or branched alkoxy group.

22. The compound according to claim 21, wherein R²¹ is a (C₁-C₆) linear or branched alkoxy group.

23. The compound according to claim 21, of formula (26), (28), (32) or (33): 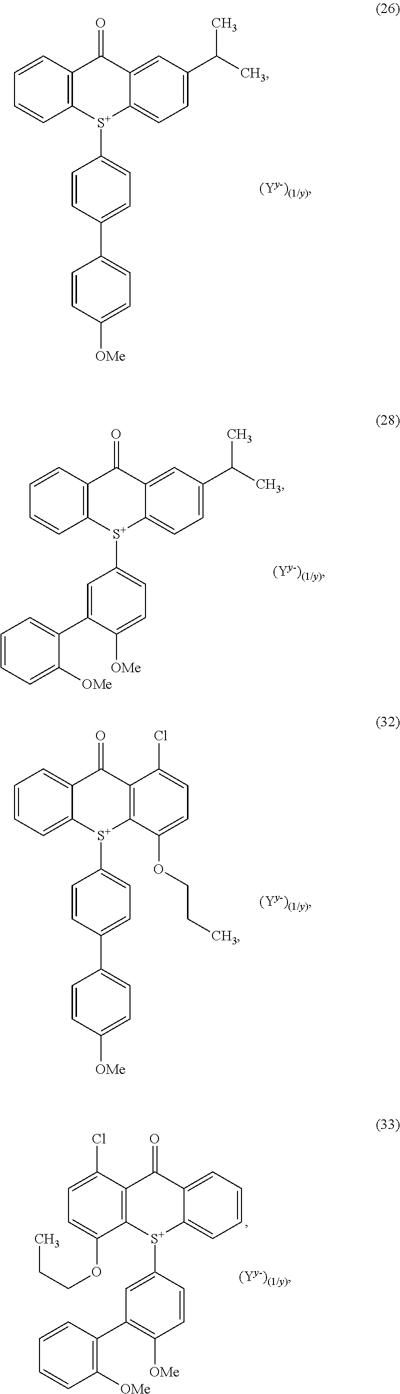
wherein Y and y are as defined in claim 21.

24. A compound of formula (V′): 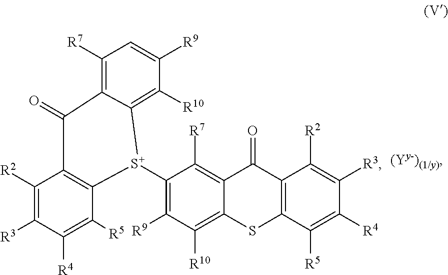
wherein:
Y is an anion, the valency of which is y,
R³, R⁴, R⁷, R⁹ and R¹⁰ are H, and
R² and R⁵ are independently chosen from a halogen, a (C₁-C₆) linear or branched alkyl group, a (C₁-C₆) linear or branched alkoxy group and a —O—(CH₂)ₘ—COOR³² or —(CH₂)ₘ—CH—(COOR³²)₂ group wherein m is 1 or 2 and R³² is H or a (C₁-C₄) linear or branched alkyl group.

25. The compound according to claim 24, of formula (5): 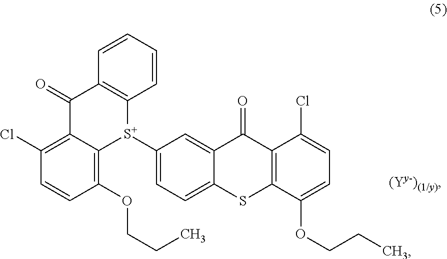
wherein Y and y are as defined in claim 24.

26. A process for the preparation of the compound of formula (V′) according to claim 24, comprising the steps of:
q) adding acetic anhydride and an acid to a mixture of a compound of formula (XX′): 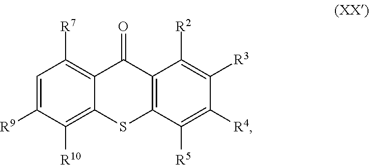
wherein R², R³, R⁴, R⁵, R⁷, R⁸, R⁹, R¹⁰ are as defined in claim 24,
and of a compound of formula (XXI′): 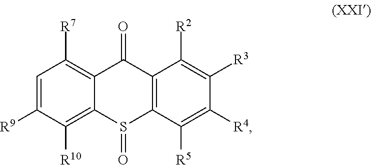
wherein R², R³, R⁴, R⁵, R⁷, R⁸, R⁹, R¹⁰ are as defined in claim 24,
whereby a photoinitiator of formula (V′) is obtained,
r) when a photoinitiator of formula (V′) is desired, wherein Y^{y-} differs from the one obtained at step q), carrying out an ion exchange reaction with a salt comprising Y′^{y-} as anion, or an acid, the base of which is Y′^{y-}, to obtain a photoinitiator of formula (V) wherein Y′^{y-} has the same definition than Y^{y-} as defined above but differs from Y^{y-} obtained at step q).

27. A curable composition comprising:
a compound of formula (VIII) according to claim 21; and
a cationically-polymerizable compound,
optionally an anti-yellowing agent.

28. The curable composition according to claim 27, wherein the compound has formula (26), (28), (32), (33) or (5).

29. The curable composition according to claim 27, comprising an anti-yellowing agent comprising an amino-group.
